(19) United States
(12) Patent Application Publication
(10) Pub. No.: US 2023/0226223 A1
(43) Pub. Date: Jul. 20, 2023

(54) Compositions and Methods for the Treatment of Protein Aggregation Disorders

(71) Applicant: SOLA BIOSCIENCES LLC, Natick, MA (US)

(72) Inventors: Akinori Hishiya, Belmont, MA (US); Keizo Koya, Chestnut Hill, MA (US)

(21) Appl. No.: 17/995,901

(22) PCT Filed: Apr. 9, 2021
(86) PCT No.: PCT/US2021/026633

§ 371 Date: Oct. 10, 2022

Related U.S. Application Data
(60) Provisional application No. 63/008,251, filed on Apr. 10, 2020.

(51) Int. Cl. A61K 48/00; C07K 14/47; A61P 25/28

(52) U.S. Cl. CPC A61K 48/0066; C07K 14/4705; A61K 48/0041; A61P 25/28; C07K 2319/70

(57) ABSTRACT

A novel class of fusion proteins to recruit a cell's innate chaperone mechanism, specifically the Hsp70-mediated system, to specifically reduce polyglutamine-mediated protein aggregation is disclosed.

### CROSS REFERENCE TO RELATED APPLICATION

This application claims priority under 35 U.S.C. § 119(e) to United States Provisional Patent Application 63/008,251, filed Apr. 10, 2020. The entire contents of the aforementioned application is hereby incorporated by reference in its entirety.

### SEQUENCE LISTING

This application incorporates by reference in its entirety the Sequence Listing entitled “269548-489649_SequenceListing_ST25.txt” (150,031 bytes) which was and last modified on Apr. 9, 2021 and filed electronically herewith.

### BACKGROUND

All proteins expressed within a cell need to correctly fold into their intended structures in order to function properly. A growing number of diseases and disorders are shown to be associated with inappropriate folding of proteins and/or inappropriate deposition and aggregation of proteins and lipoproteins as well as infectious proteinaceous substances. Also known as a conformational disease or proteopathy, examples of diseases caused by misfolding include cystic fibrosis (CF), polyglutamine repeat disorders, Parkinson's disease (PD), and Alzheimer's disease (AD). The mutant protein aggregates in cells causing typical cytotoxic cellular inclusion bodies.

Disorders identified as protein repeat expansion disorders contain expansions of a homopolymeric stretch of amino acids, often a stretch of glutamine residues, or polyglutamine (poly Q). At least eight neurodegenerative disorders have been associated with polyglutamine expansions, including Huntington's disease (HD), spinal and bulbar muscular atrophy (SBMA), dentatorubral and pallidoluysian atrophy (DRPLA), and several forms of spinocerebellar ataxia (SCA).

One common physiological characteristic shared among these genetically distinct diseases is that patients who suffer from the diseases are all found to have proteinaceous deposits in their brains. Although in each of these diseases, the proteinaceous deposit is associated with a different protein, the proteins all contain an expanded stretch of glutamines. To date, this expanded stretch of polyglutamine (polyQ) sequence in the disease-related proteins is the only known genetic mutation implicated in all the polyglutamine repeat diseases.

Huntington's disease (HD) is a hereditary polyglutamine repeat disease characterized by selective neuronal cell loss and astrocytosis mainly in the cerebral cortex and corpus striatum (Vonsattel 2007). Current drug therapy under investigation is limited to either the treatment of characteristic motor impairment with antichoreic/neuroleptic drugs or the reduction in expression of huntingtin (either indiscriminate or selective to the mutant form), but there is no causative treatment to affect the progressive nature of the disease including dementia and psychiatric disturbances (Bonelli, 2007).

HD is caused by an unstable CAG repeat expansion in the first exon of the huntingtin gene (IT-15) which translates into an elongated polyglutamine repeat (polyQ) stretch in the protein huntingtin. A pathological polyQ length of more than 37 glutamine residues is associated with the appearance of cytosolic, perinuclear and nuclear inclusions containing amino-terminal huntingtin fragments and sequestered proteins (Imarisio et al., 2008).

Currently, available treatments for HD are mainly limited to managing the macroscopic symptoms. For example, one of the newest compounds approved by the FDA, tetrabenazine, is a drug for reducing hyperkinetic movements in HD patients. Tetrabenazine is a vesicular monoamine transporter (VMAT) inhibitor which promotes early degradation of neurotransmitters. Thus, the drug merely treats the symptom, not the root of the disease. Other drugs currently used for treating HD include neuroleptics and benzodiazepines. No presently known treatment is attempting to address the root cause of HD. There are no approved therapies for the treatment of other polyglutamine repeat diseases.

Therefore, a need exists for the development of new therapeutic modalities optimized to target specific antigens, proteins, glycoproteins or lipoproteins, particularly against diseases with pathologies based on protein misfolding and aggregation, as well as in cases of heterogeneous aggregates.

The heat shock 70 kDa proteins (referred to herein as “Hsp70s”) constitute a ubiquitous class of chaperone proteins in the cells of a wide variety of species (Tavaria et al., (1996) Cell Stress Chaperones 1, 23-28). Hsp70 requires assistant proteins called co-chaperone proteins, such as i domain proteins and nucleotide exchange factors (NEFs) (Hartl et al., (2009) Nat Struct Mol Biol 16, 574-581), in order to function. In the current model of Hsp70 chaperone machinery for folding proteins, Hsp70 cycles between ATP- and ADP-bound states, and a J domain protein binds to another protein in need of folding or refolding (referred to as a “client protein”), interacting with the ATP-bound form of Hsp70 (Hsp70-ATP) (Young (2010) Biochem Cell Biol 88, 291-300; Mayer, (2010) Mol Cell 39, 321-331). Binding of the J domain protein-client complex to Hsp70-ATP stimulates ATP hydrolysis, which causes a conformational change in the Hsp70 protein, closing a helical lid and, thereby, stabilizing the interaction between the client protein with Hsp70-ADP, as well as eliciting the release of the J domain protein that is then free to bind to another client protein.

Therefore, according to this model, J domain proteins play a critical part within the Hsp70 machinery by acting as a bridge, facilitating the capture and recruitment of a wide variety of client proteins into the Hsp70 machinery to promote folding or refolding into the proper conformation (Kampinga & Craig (2010) Nat Rev Mol Cell Biol 11, 579-592). The J domain family is widely conserved in species ranging from prokaryotes (DnaJ protein) to eukaryotes (Hsp40 protein family). The J domain (about 60-80 aa) is composed of four helices: I, II, III, and IV. Helices II and III are connected via a flexible loop containing an “HPD motif”, which is highly conserved across J domains and thought to be critical for activity (Tsai & Douglas, (1996) J Biol Chem 271, 9347-9354). Mutations within the HPD sequence has been found to abolish J domain function.

Given the context provided above for proteopathies such as Huntington's disease, it seems clear that reducing the level of misfolded proteins could serve as a means to treat, prevent or otherwise ameliorate the symptoms of these devastating disorders and that, recruitment of a cell's innate ability to repair protein misfolding would be a logical choice to pursue. In fact, a number of attempts at developing a therapeutic based on chaperone have been suggested to be a promising strategy for these diseases. However, these therapeutic applications have often been found to be associated with unwanted outcomes, presumably due to the relative promiscuity of chaperones with respect to the client proteins. For example, the overwhelming majority of human tumors overexpress HSP70 and overexpression of HSP70 has been found to lead to an increased risk of carcinogenesis and a poor prognosis in cancer patients (Murphy (2013) Carcinogenesis, 34:1181-1188). Likewise, application of chaperone modifiers can result in the inhibition of enzymes closely related to the target protein, presumably due to lack of selectivity (Pereira et al., (2018) Chem. Sci., 2018, 9, 1740-1752). Therefore, successful development of chaperone-based therapies would need to provide specificity towards the pathological proteins. As such, a need exists to develop highly specific chaperones for the treatment of proteopathies.

### SUMMARY OF THE INVENTION

The inventors have developed a novel class of fusion proteins to recruit a cell's innate chaperone mechanism, specifically the Hsp70-mediated system, to specifically reduce polyglutamine-mediated protein aggregation. Unlike in previous studies by the inventors using fusion proteins comprising fragments of a Hsp40 protein (also called J proteins), a co-chaperone that interacts with Hsp70, to enhance protein secretion and expression, the present study employs J domain-containing fusion proteins for the purpose of reducing protein aggregation and cytotoxicity caused by intracellular proteins containing polyglutamine repeats. In this context, the inventors have made the surprising discovery that the elements of J domain required for function is quite distinct from use of J domains in enhancing protein expression and secretion, demonstrating a distinct mechanism for the mode of action of the present fusion proteins. The fusion proteins described herein comprise a J domain and a domain that has affinity for polyglutamine repeats. The presence of the polyglutamine-binding domain within the fusion protein results in specific reduction in aggregation of proteins with polyglutamine repeats.
E1. Therefore, in a first aspect, disclosed herein is an isolated fusion protein comprising a J domain of a J protein and a polyglutamine-binding domain.
E2. The fusion protein of E1, wherein the J domain of a J protein is of eukaryotic origin.
E3. The fusion protein of any one of E1-E2, wherein the i domain of a J protein is of human origin.
E4. The fusion protein of any one of E1-E3, wherein the J domain of a J protein is cytosolically localized.
E5. The fusion protein of any one of E1-E4, wherein the i domain of a J protein is selected from the group consisting of SEQ ID Nos: 1-50.
E6. The fusion protein of any one of E1-E5, wherein the J domain comprises the sequence selected from the group consisting of SEQ ID NOs: 1, 5, 6, 10, 24, 25, 31 and 49.
E7. The fusion protein of any one of E1-E6, wherein the J domain comprises the sequence of SEQ ID NO: 5.
E8. The fusion protein of any one of E1-E6, wherein the J domain comprises the sequence of SEQ ID NO: 10.
E9. The fusion protein of any one of E1-E6, wherein the J domain comprises the sequence of SEQ ID NO: 24.
E10. The fusion protein of any one of E1-E6, wherein the J domain comprises the sequence of SEQ ID NO: 31.
E11. The fusion protein of any one of E1-E6, wherein the J domain comprises the sequence of SEQ ID NO: 49.

E12. The fusion protein of any one of E1-E11, wherein the polyglutamine-binding domain has a K_{D} for polyglutamine repeats (for example, a thioredoxin-Q₆₂ construct) of 2 μM or less, for example, 1 μM or less, 500 nM or less, 300 μM or less, 200 nM or less when tested using an indirect assay measuring inhibition of thioredoxin-Q₆₂ aggregation.
E13. The fusion protein of any one of E1-E12, wherein the polyglutamine-binding domain comprises the sequence selected from the group consisting of SEQ ID NOs: 51-68.
E14. The fusion protein of any one of E1-E13, wherein the polyglutamine-binding domain comprises the sequence of SEQ ID NO:57, SEQ ID NO:62 or SEQ ID NO:68.
E15. The fusion protein of any one of E1-E14, comprising a plurality of polyglutamine-binding domains.
E16. The fusion protein of any one of E1-E15, consisting of two polyglutamine-binding domains.
E17. The fusion protein of any one of E1-E15, consisting of a plurality of J domains.
E18. The fusion protein of any one of E1-E17, comprising one of the following constructs:
   a. DNAJ-X-Q,
   b. DNAJ-X-Q-X-Q,
   c. DNAJ-X-Q-X-Q-X-Q,
   d. Q-X-DNAJ,
   e. Q-X-Q-X-DNAJ,
   f. Q-X-Q-X-Q-X-DNAJ,
   g. Q-X-DNAJ-X-Q,
   h. Q-X-DNAJ-X-Q-X-Q,
   i. DNAJ-X-DNAJ-X-Q,
   j. Q-X-Q-X-DNAJ-X-Q,
   k. DNAJ-X-Q-X-DNAJ-X-Q,
   l. Q-X-Q-X-DNAJ-X-Q-X-Q-X-Q,
   m. Q-X-Q-X-Q-X-DNAJ-X-Q,
   n. Q-X-Q-X-Q-X-DNAJ-X-Q-X-Q,
   o. Q-X-Q-X-Q-X-DNAJ-X-Q-X-Q-X-Q,
   p. DnaJ-X-DnaJ-X-Q-X-Q,
   q. Q-X-DnaJ-X-DnaJ,
   r. Q-X-Q-X-DnaJ-X-DnaJ, and
   s. Q-X-DnaJ-X-DnaJ-X-Q
   wherein,
   Q is a polyglutamine-binding domain,
   DNAJ is a J domain of a J protein, and
   X is an optional linker.
E19. The fusion protein of any one of E1-E18, wherein the fusion protein comprises the J domain sequence of SEQ ID NO: 5 and the polyglutamine-binding domain sequence of SEQ ID NO: 57.
E20. The fusion protein of any one of E1-E19, wherein the fusion protein comprises the J domain sequence of SEQ ID NO: 5 and two copies of the polyglutamine-binding domain sequence of SEQ ID NO: 57.
E21. The fusion protein of any one of E1-E19, wherein the fusion protein comprises the J domain sequence of SEQ ID NO: 5 and three copies of the polyglutamine-binding domain sequence of SEQ ID NO: 57.
E22. The fusion protein of any one of E1-E21, wherein the fusion protein comprises the sequence selected from the group consisting of SEQ ID Nos: 89-158.
E23. The fusion protein of any one of E1-E22, wherein the fusion protein comprises the sequence selected from the group consisting of SEQ ID Nos: 90, 122, 139, 140, 141, 157 and 158.
E24. The fusion protein of any one of E1-E22, wherein the fusion protein comprises the sequence of SEQ ID NO: 122.
E25. The fusion protein of any one of E1-E22, wherein the fusion protein comprises the sequence of SEQ ID NO: 139.
E26. The fusion protein of any one of E1-E22, wherein the fusion protein comprises the sequence of SEQ ID NO: 140.
E27. The fusion protein of any one of E1-E22, wherein the fusion protein comprises the sequence of SEQ ID NO: 141.
E28. The fusion protein of any one of E1-E22, wherein the fusion protein comprises the sequence of SEQ ID NO: 90.
E29. The fusion protein of any one of E1-E22, wherein the fusion protein comprises the sequence of SEQ ID NO: 157.
E30. The fusion protein of any one of E1-E22, wherein the fusion protein comprises the sequence of SEQ ID NO: 158.
E31. The fusion protein of any one of E1-E30, further comprising a targeting reagent.
E32. The fusion protein of any one of E1-E31, further comprising an epitope.
E33. The fusion protein of any one of E1-E32, further comprising a cell-penetrating agent.
E34. The fusion protein of E33, wherein the cell-penetrating agent comprises a peptide sequence selected from the group consisting of SEQ ID NOs: 85-88.
E35. The fusion protein of any one of E1-E34, further comprising a signal sequence.
E36. The fusion protein of E35, wherein the signal sequence comprises the peptide sequence selected from the group consisting of SEQ ID NOs: 159-161.
E37. The fusion protein of any one of E1-E36, which is capable of reducing aggregation of pathogenic proteins in a cell.
E38. The fusion protein of any one of E1-E37, which is capable of reducing polyglutamine repeat-mediated cytotoxicity.
E39. A nucleic acid sequence encoding the fusion protein of any one of E1-E38.
E40. The nucleic acid sequence of E39, wherein said nucleic acid is DNA.
E41. The nucleic acid sequence of any one of E39-E40, wherein said nucleic acid is RNA.
E42. The nucleic acid sequence of any one of E39-E41, wherein said nucleic acid comprises at least one modified nucleic acid.
E43. The nucleic acid sequence of any one of E39-E42, further comprising a promoter region, 5′ UTR, 3′ UTR such as poly(A) signal.
E44. The nucleic acid sequence of E43, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.
E45. A vector comprising the nucleic acid sequence of any one of E39-E44.
E46. The vector of E45, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.
E47. The vector of E45 or E46, wherein the vector is an AAV.
E48. A virus particle comprising a capsid and the vector of E41 or E42.
E49. The virus particle of E48, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-Dian AAV capsid mutant, an AAV hybrid serotype, an organ-tropic AAV, a cardiotropic AAV, and a cardiotropic AAVM41 mutant.
E50. The virus particle of E48 or E49, wherein the capsid is selected from the group consisting of AAV2, AAV5, AAV8, AAV9 and AAVrh10.
E51. The virus particle of any one of E48-E50, wherein the capsid is AAV2.
E52. The virus particle of any one of E48-E50, wherein the capsid is AAV5.
E53. The virus particle of any one of E48-E50, wherein the capsid is AAV8.
E54. The virus particle of any one of E48-E50, wherein the capsid is AAV9.
E55. The virus particle of any one of E48-E50, wherein the capsid is AAV rh10.
E56. A pharmaceutical composition comprising an agent selected from the group consisting of the fusion protein of any one of E1-E38, a cell expressing the fusion protein of E1-E38, the nucleic acid of any one of E39-E44, the vector of any one of E41-E45-E47, the virus particle of any one of E48-E55, and a pharmaceutically acceptable carrier or excipient.
E57. A method of reducing toxicity of a polyglutamine protein in a cell, comprising contacting said cell with an effective amount of one or more agents selected from the group consisting of: the fusion protein of any one of E1-E38, a cell expressing the fusion protein of E1-E38, the nucleic acid of any one of E39-E44, the vector of any one of E45-E47, the virus particle of any one of E48-E55, and the pharmaceutically composition of E56.
E58. The method of E57, wherein the cell is in a subject.
E59. The method of E57 or E58, wherein the subject is a human.
E60. The method of any one of E57-E59, wherein the cell is a cell of the central nervous system.
E61. The method of any one of E57-E60, wherein subject is identified as having a polyglutamine repeat disease.
E62. The method of any one of E57-E61, wherein the polyglutamine protein is selected from the group consisting of huntingtin, atrophin-1, ataxin 1, ataxin 2, Cav2.1, ataxin 7, TATA-binding protein, ataxin 3, and androgen receptor.
E63. The method of any one of E57-E62, wherein there is a reduction in aggregation of the polyglutamine protein in the cell.
E64. A method of treating, preventing, or delaying the progression of a polyglutamine repeat disease in a subject in need thereof, the method comprising administering an effective amount of one or more agents selected from the group consisting of with the fusion protein of any one of E1-E38, a cell expressing the fusion protein of E1-E38, the nucleic acid of any one of E39-E44, the vector of any one of E45-E47, the virus particle of any one of E48-E55, and the pharmaceutically composition of E56.
E65. The method of E64, wherein the polyglutamine repeat disease is selected from the group consisting of Huntington's disease, SCA type 1, SCA type 2, SCA type 6, SCA type 7, SCA type 17, MJD/SCA3, DRPLA, and SBMA.
E66. Use of one or more of the fusion protein of any one of E1-E38, a cell expressing the fusion protein of E1-E38, the nucleic acid of any one of E39-E44, the vector of any one of E45-E47, the virus particle of any one of E48-E55, and the pharmaceutically composition of E56 for use in preventing or delaying the progression of a polyglutamine repeat disease in a subject.
E67. A method of reducing protein aggregation in a cell, comprising contacting said cell with an effective amount of one or more agents selected from the group consisting of: the fusion protein of any one of E1-E38, a cell expressing the fusion protein of E1-E38, the nucleic acid of any one of E39-E44, the vector of any one of E45-E47, the virus particle of any one of E48-E55, and the pharmaceutically composition of E56.
E68. The method of E67, wherein the cell is in a subject.
E69. The method of any one of E67-E68, wherein the subject is a human.
E70. The method of any one of E67-E69, wherein the cell is a cell of the central nervous system.
E71. The method of any one of E67-E70, wherein subject is identified as having a disease selected from the group consisting of ALS, FTD, Parkinson's disease, Huntington's disease, Alzheimer's disease, hippocampal sclerosis, prion disease, and dementia with Lewy's bodies.
E72. The method of any one of E67-E71, wherein there is a reduction in aggregation of the protein in the cell.
E73. A method of treating, preventing, or delaying the progression of a protein aggregation disease in a subject in need thereof, the method comprising administering an effective amount of one or more agents selected from the group consisting of with the fusion protein of any one of E1-E38, a cell expressing the fusion protein of E1-E38, the nucleic acid of any one of E39-E44, the vector of any one of E45-E47, the virus particle of any one of E48-E55, and the pharmaceutically composition of E56.
E74. The method of E73, wherein the polyglutamine repeat disease is selected from the group consisting of Huntington's disease, SCA type 1, SCA type 2, SCA type 6, SCA type 7, SCA type 17, MJD/SCA3, DRPLA, and SBMA.
E75. Use of one or more of the fusion protein of any one of E1-E38, a cell expressing the fusion protein of E1-E38, the nucleic acid of any one of E39-E44, the vector of any one of E45-E47, the virus particle of any one of E48-E55, and the pharmaceutically composition of E56, for use in preventing or delaying the progression of a protein aggregation disease in a subject.

### DESCRIPTION OF THE FIGURES

FIG. 1A shows a Clustal Omega sequence alignment of representative human J domain sequences. The highly conserved HPD domain is shown in the highlighted box.

FIG. 1B shows a Clustal Omega sequence alignment of representative human J domain sequences.

FIG. 2 shows some illustrative fusion protein constructs comprising a J domain and polyglutamine-binding domains.

FIG. 3 shows the effect on protein aggregation as measured by filter trap assay of expressing fusion proteins (Constructs 1-7) in cells also expressing a reporter construct containing a huntingtin protein fragment with either the normal level (23 repeats in GFP-HTTQ23) or mutant level (74 repeats in GFP-HTTQ74) of polyglutamine expansion.

FIG. 4 shows the ability of additional fusion protein constructs with modified J domains to reduce huntingtin aggregation in cells expressing the huntingtin reporter construct GFP-HTTQ74.

FIG. 5 shows a comparison of different linkers within fusion protein constructs.

FIG. 6 shows a reduction of protein aggregation in cells expressing the J-2XQBP1 construct (FIG. 6c) as measured by fluorescence microscopy, and compared with GFP-HTTQ74 alone (FIG. 6a), GFP-HTTQ23 alone (FIG. 6b), or GFP-HTTQ74 co-expressing JB1(P33Q)-2XQBP1, a fusion protein with a mutation within the conserved HPD motif (FIG. 6d).

FIG. 7 shows additional constructs tested for the ability to reduce protein aggregation as measured by fluorescence microscopy (FIG. 7A) and the filter trap assay (FIG. 7B).

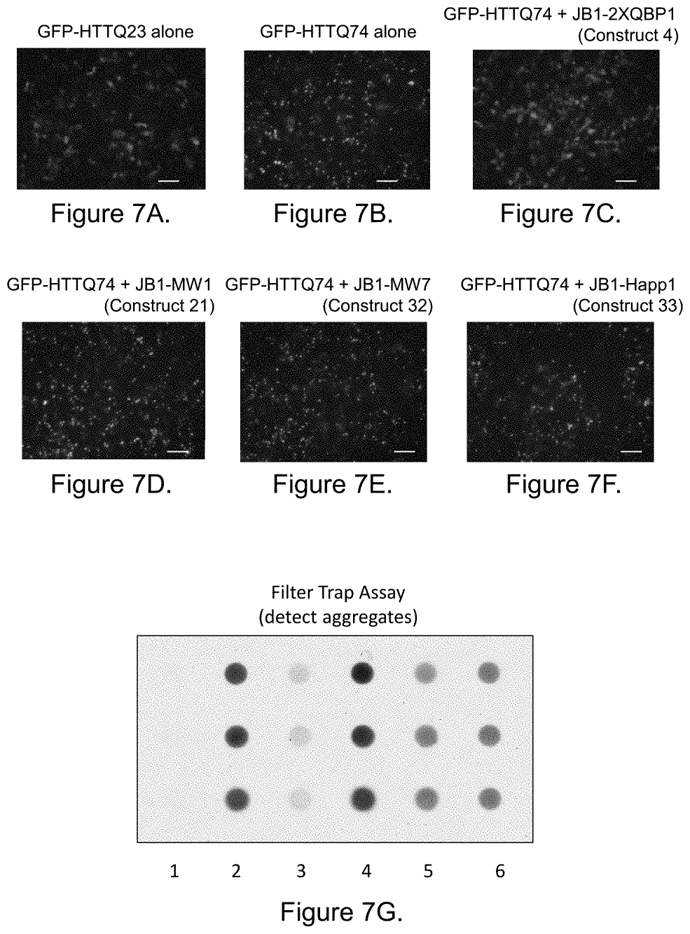

FIG. 8 Shows the effect of expressing Construct 56 (JB1-JB1-QBP1) in U87-MG glioma cells. A. U87-MG glioma cells were infected with lentivirus to express Myc-tagged normal HTT (Q23) or mHTT (Q74) with or without Construct 56. Culture medium was replaced with fresh medium at day 2 and 4. Cells were lysed at day 7 for filter trap assay. 0.5 μg or 2.5 μg of total protein was applied to the filter trap assay apparatus, followed by immunoblotting assay using anti-HTT antibody (MW8). B. The culture medium was collected from U87-MG cells at 7-day after infection. Lactate dehydrogenase (LDH) activity in culture medium was measured by LDH-Cytox™ Assay Kit (BioLegend). Values represent the mean±SD. *p<0.05.

FIG. 9 Shows the effect of ICV administration of control or Construct 53-expressing rAAV capsids (in AAV rh10) in wild-type mice. A. Body weight for each mouse was recorded daily following the ICV AAV injection. B. Frozen cortex sections were stained with anti-Flag antibody (1,4,green) and anti-NeuN antibody (2,5, red), and counterstained with Dapi (3,6, blue). Images are representative cortical areas. Panels 1-3: Injection with control AAVrh10; Panels 4-6, Injection with Flag-QBP1-JB1-QBP1-Flag AAVrh10.

### DEFINITIONS

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to both the D or L optical isomers, and amino acid analogs and peptidomimetics. Standard single or three letter codes are used to designate amino acids.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “polyglutamine disorder” or “polyglutamine repeat disorder”, as herein defined refers to disorders associated with formation of intracellular polyglutamine aggregates, preferably referring to Huntington's disease (HD), spinal and bulbar muscular atrophy (SBMA), dentatorubral-pallidoluysian atrophy (DRPLA) and spinocerebellar ataxia (SCA) type 1, SCA type 2, SCA type 6, SCA type 7, SCA type 17, or MJD/SCA3.

Likewise, the term “polyglutamine-containing protein” refers to a protein which contains a stretch of at least 10, for example, at least 13, at least 15, at least 20, at least 25, at least 30 or more glutamine amino acids. In many cases, a polyglutamine-containing protein contains abnormally high levels or polyglutamine when compared with a wild-type protein. Proteins containing polyglutamine stretches include, but not limited to, huntingtin, atrophin-1, ataxin 1, ataxin 2, Cav2.1, ataxin 7, TATA-binding protein, ataxin 3, and androgen receptor.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “operably linked” refers to a juxtaposition of described components wherein the components are in a relationship permitting them to function in their intended manner. A control sequence “operably linked” to a coding sequence is ligated in such a way that expression of the coding sequence is achieved under conditions compatible with the control sequences. “Operably linked” sequences may include both expression control sequences that are contiguous with the gene of interest and expression control sequences that act in trans or at a distance to control the gene of interest. The term “expression control sequence” refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (such as, a Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing and can also include additional components whose presence is advantageous, for example, leader sequences and fusion partner sequences. Unless stated otherwise, a description or statement herein of inserting a nucleic acid molecule encoding a fusion protein of the invention into an expression vector means that the inserted nucleic acid has also been operably linked within the vector to a functional promoter and other transcriptional and translational control elements required for expression of the encoded fusion protein when the expression vector containing the inserted nucleic acid molecule is introduced into compatible host cells or compatible cells of an organism.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

The terms “disease” and “disorder” are used interchangeably to indicate a pathological state identified according to acceptable medical standards and practices in the art.

As used herein, the term “effective amount” refers to the amount of a therapy that is sufficient to reduce or ameliorate the severity and/or duration of a disease or one or more symptoms thereof; to prevent the advancement of a detrimental or pathological state; to cause regression of a pathological state; to prevent recurrence, development, onset, or progression of one or more symptoms associated with a pathological state; to detect a disorder; or to enhance or improve the prophylactic or therapeutic effect(s) of a therapy (e.g., the administration of another prophylactic or therapeutic agent).

As used herein, the term “J domain” refers to a fragment which retains the ability to accelerate the intrinsic ATPase catalytic activity of Hsp70 and its cognate. The J domains of a variety of J proteins have been determined (see, for example, Kampinga et al. (2010) Nat. Rev., 11: 579-592; Hennessy et al. (2005) Protein Science, 14: 1697-1709, each of which is incorporated by reference in its entirety), and are characterized by a number of hallmarks: which is characterized by four α-helices (I, II, III, IV) and usually having the highly conserved tripeptide sequence motif of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is necessary for stimulation of Hsp70 ATPase activity. As used herein, the term “J domain” is meant to include natural J domain sequences and functional variants thereof which retain the ability to accelerate Hsp70 intrinsic ATPase activity, which can be measured using methods well known in the art (see, for example, Horne et al. (2010) J. Biol. Chem., 285, 21679-21688, which is incorporated herein by reference in its entirety). A non-limiting list of human J domains is provided in Table 1.

### DETAILED DESCRIPTION

The present inventors have found that certain contacting cells with a fusion protein construct comprising a J domain of a J protein and a polyglutamine-binding domain have the unexpected effect of reducing the aggregation of polyglutamine-containing proteins. Aggregation of such proteins containing polyglutamine repeats are believed to cause a number of devastating diseases, including, but not limited to, Huntington's Disease, Spinocerebellar Ataxia (SCA) type 1, SCA type 2, SCA type 6, SCA type 7, SCA type 17, MJD/SCA3, Dentatorubral-pallidoluysian atrophy (DRPLA), and Spinal and bulbar muscular atrophy (SBMA). Accordingly, useful compositions and methods to treat polyglutamine repeat disorders, e.g., in a subject in need thereof, are provided herein.

To overcome issues associated with chaperone-based therapies, we investigated whether it would be possible to design artificial chaperone proteins with high specificity. We designed a series of fusion protein constructs comprising an effector domain for Hsp70 binding/activation (J domain sequence), and a domain conferring specificity to proteins with polyglutamine repeats. The resulting fusion proteins act to accelerate the intrinsic ATPase catalytic activity of Hsp70 and its cognate, resulting in increased protein folding and reduced aggregation.

I. Fusion Protein Constructs

a. J Domains Useful in the Invention

J domains of a variety of J proteins have been determined. See, for example, Kampinga et al., Nat. Rev., 11: 579-592 (2010); Hennessy et al., Protein Science, 14:1697-1709 (2005). A J domain useful in preparing a fusion protein of the invention has the key defining features of a J domain which principally accelerates HSP70 ATPase activity. Accordingly, an isolated J domain useful in the invention comprises a polypeptide domain, which is characterized by four α-helices (I, II, III, IV) and usually having the highly conserved tripeptide sequence of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is fundamental to primitive activity. Representative J domains include, but are not limited, a J domain of an DnaJB1, DnaJB2, DnaJB6, DnaJC6, a J domain of a large T antigen of SV40, and a J domain of a mammalian cysteine string protein (CSP-α). The amino acid sequences for these and other J domains that may be used in fusion proteins of the invention are provided in Table 1. The conserved HPD motif is highlighted in bold. Preliminary experiments performed confirm the essentiality of the HPD motif: indeed, fusion protein constructs using the J domain from DNA JB13 (SEQ ID NO:16) was found not to be capable of reducing protein aggregation (Construct 59). Therefore, in one embodiment, the fusion protein comprises a J domain sequence comprising an HPD domain. In one embodiment, the fusion protein comprises a J domain of a J protein that is selected from the group consisting of SEQ ID Nos: 1-15, 17-50. In another embodiment, the fusion protein comprises a J domain of a J protein that is selected from the group consisting of SEQ ID NOs: 1, 5, 6, 10, 24, 25, 31 and 49. In one embodiment, the fusion protein comprises the J domain of SEQ ID NO: 5. In one embodiment, the fusion protein comprises the J domain of SEQ ID NO: 10. In another embodiment, the fusion protein comprises the J domain of SEQ ID NO: 24. In yet another embodiment, the fusion protein comprises the J domain of SEQ ID NO: 31. In still another embodiment, the fusion protein comprises the J domain of SEQ ID NO: 49.

TABLE 1
Representative Human J Domain Sequences

| Protein | SEQ ID | Gene NCBI | Protein NCBI | |
|---|---|---|---|---|
| Name | NO: | Reference | Reference | J domain amino acid sequence |
| DNAJA1 | 1 | NM_001539 | NP_001530 | TYYDVLGVKPNATQEELKKAYRK |
| | | | | LALKYHPDKNPNEGEKFKQISQA |
| | | | | YEVLSDAKKRELYDKGG |
| DNAJA2 | 2 | NM_005880 | NP_005871 | KLYDILGVPPGASENELKKAYRK |
| | | | | LAKEYHPDKNPNAGDKFKEISFA |
| | | | | YEVLSNPEKRELYDRYG |
| DNAJA3 | 3 | NM_001135110 | NP_001128582 | DYYQILGVPRNASQKEIKKAYYQ |
| | | | | LAKKYHPDTNKDDPKAKEKFSQL |
| | | | | AEAYEVLSDEVKRKQYDAYG |
| DNAJA4 | 4 | NM_001130182 | NP_001123654 | ETQYYDILGVKPSASPEEIKKAY |
| | | | | RKLALKYHPDKNPDEGEKFKLIS |
| | | | | QAYEVLSDPKKRDVYDQGGEQ |
| DNAJB1 | 5 | NM_001300914 | NP_001287843 | GRDYYQTLGLARGASDEEIRRAY |
| | | | | RRQALRYHPDRNREPGAEERFRE |
| | | | | IAEAYDVLSDPRRREIFDRYGEE |
| DNAJB2 | 6 | NM_001039550 | NP_001034639 | ASYYEILDVPRSASADDIKKAYR |
| | | | | RKALQWHPDKNPDNKEFAEKKFK |
| | | | | EVAEAYEVLSDRHRREIYDRYGR |
| | | | | E |
| DNAJB3 | 7 | NM_001001394 | NP_001001394 | MVDYYEVLDVPRQASSEAIRRAY |
| | | | | RKLALKWHPDKNPENKEEAERRF |
| | | | | KQVAEAYEVLSDAKKRDIYDRYG |
| DNAJB4 | 8 | NM_001317099 | NP_001304028 | GKDYYCILGIEKGASDEDIKKAY |
| | | | | RKQALKFHPDKNKSPQAEERFRE |
| | | | | VAEAYEVLSDPRRREIYDQFGEE |
| DNAJB5 | 9 | NM_001135004 | NP_001128476 | DYYKILGIPSGANEDEIKKAYRK |
| | | | | MALKYHPDKNKEPNAEEKFKEIA |
| | | | | EAYDVLSDPKKRGLYDQYG |
| DNAJB6 | 10 | NM_005494 | NP_005485 | VDYYEVLGVQRHASPEDIKKAYR |
| | | | | KLALKWHPDKNPENKEEAERKFK |
| | | | | QVAEAYEVLSDAKKRDIYDRYG |
| DNAJB7 | 11 | NM_145174 | NP_660157 | DYYEVLGLQRYASPEDIRRAYHR |
| | | | | VALRWHPDRNPENREEAERRFRE |
| | | | | VAEAYEVLSNDERRDIYDRYG |
| DNAJB8 | 12 | NM_153330 | NP_699161 | NYYEVLGVQASASPEDIRRAYRR |
| | | | | LALRWHPDRNPDNREEAERRFRL |
| | | | | VSEAYEVLSDSRRRSLYDRAG |
| DNAJB9 | 13 | NM_012328 | NP_036460 | SYYDILGVPRSASERQIRRAFHR |
| | | | | LAMRYHPDRNRSPDAEARFREIA |
| | | | | EAYETLSDANRRREYDTLG |
| DNAJB11 | 14 | NM_016306 | NP_057390 | DFYRILGVPRSASIRDIRRAYRR |
| | | | | LALQLHPDRNPDDPQAQERFQDL |
| | | | | GAAYEVLSDSERRRQYDTYG |
| DNAJB12 | 15 | NM_001002762 | NP_001002762 | YEILGVSRGASDEDLRRAYRRLA |
| | | | | LRFHPDRNHAPGATEAFRAIGTA |
| | | | | YAVLSNPERRRQYDQFGDD |
| DNAJB13 | 16 | NM_153614 | NP_705842 | DYYSVLGITRNSEDAQIRQAYRR |
| | | | | LALRHHPLRSNEPSSAEIFRQIA |
| | | | | EAYDVLSDPMRRGIYDRFG |
| DNAJB14 | 17 | NM_001031723 | NP_001026893 | NYYEVLGVTRDAGDEDLRRAYRR |
| | | | | LALRFHPDRNHAPGATDAFRRIG |
| | | | | NAYAVLSNPERRRQYDLTG |
| DNAJC1 | 18 | NM_022365 | NP_071760 | NFYQFLGVQQDASSADIRRAYRR |
| | | | | LSLTLHPDRNRDENAETQFRQLV |
| | | | | AIYEVLRDDERRQRYDDIL |
| DNAJC2 | 19 | NM_001129887 | NP_001123359 | DHYAVLGLGHVRYRATQRQIRAA |
| | | | | HRAMVLRHHPDRRRAAGEPIREG |
| | | | | DNDYFTCITKAYEMLSDPVKRRA |
| | | | | FNSVD |
| DNAJC3 | 20 | NM_006260 | NP_006251 | DYYKILGVKRNAKKQE11KAYRK |
| | | | | LALQWHPDNFQNEEEKKKAEKKF |
| | | | | IDIAAAKEVLSDPEMRKKFDDGE |
| DNAJC4 | 21 | NM_001307980 | NP_001294909 | TYYELLGVHPGASTEEVKRAFFS |
| | | | | KSKELHPDRDPGNPSLHSRFVEL |
| | | | | SEAYRVLSREQSRRSYDDQL |
| DNAJC5 | 22 | NM_025219 | NP_079495 | GESLYHVLGLDKNATSDDIKKSY |
| | | | | RKLALKYHPDKNPDNPEAADKFK |
| | | | | EINNAHAILTDATKRNIYDKYGS |
| | | | | L |
| DNAJC5B | 23 | NM_033105 | NP_149096 | ALYEILGLHKGASNEEIKKTYRK |
| | | | | LALKHHPDKNPDDPAATEKFKEI |
| | | | | NNAHAILTDISKRSIYDKYG |
| DNAJC6 | 24 | NM_001256864 | NP_001243793 | TKWKPVGMADLVTPEQVKKVYRK |
| | | | | AVLVVHPDKATGQPYEQYAKMIF |
| | | | | MELNDAWSEFENQGQKPLY |
| DNAJC7 | 25 | NM_001144766 | NP_001138238 | DYYKILGVDKNASEDEIKKAYRK |
| | | | | RALMHHPDRHSGASAEVQKEEEK |
| | | | | KFKEVGEAFTILSDPKKKTRYDS |
| | | | | GQ |
| DNAJC8 | 26 | NM_014280 | NP_055095 | NPFEVLQIDPEVTDEEIKKRFRQ |
| | | | | LSILVHPDKNQDDADRAQKAFEA |
| | | | | VDKAYKLLLDQEQKKRALDVIQ |
| DNAJC9 | 27 | NM_015190 | NP_056005 | DLYRVLGVRREASDGEVRRGYHK |
| | | | | VSLQVHPDRVGEGDKEDATRRFQ |
| | | | | ILGKVYSVLSDREQRAVYDEQG |
| DNAJC10 | 28 | NM_001271581 | NP_001258510 | DFYSLLGVSKTASSREIRQAFKK |
| | | | | LALKLHPDKNPNNPNAHGDFLKI |
| | | | | NRAYEVLKDEDLRKKYDKYG |
| DNAJC11 | 29 | NM_018198 | NP_060668 | DYYSLLNVRREASSEELKAAYRR |
| | | | | LCMLYHPDKHRDPELKSQAERLF |
| | | | | NLVHQAYEVLSDPQTRAIYDIYG |
| DNAJC12 | 30 | NM_021800 | NP_068572 | DYYTLLGCDELSSVEQILAEFKV |
| | | | | RALECHPDKHPENPKAVETFQKL |
| | | | | QKAKEILTNEESRARYDHWR |
| DNAJC13 | 31 | NM_015268 | NP_056083 | DAYEVLNLPQGQGPHDESKIRKA |
| | | | | YFRLAQKYHPDKNPEGRDMFEKV |
| | | | | NKAYEFLCTKSAKIVDGPDP |
| DNAJC14 | 32 | NM_032364 | NP_115740 | NPFHVLGVEATASDVELKKAYRQ |
| | | | | LAVMVHPDKNHHPRAEEAFKVLR |
| | | | | AAWDIVSNAEKRKEYEMKR |
| DNAJC15 | 33 | NM_013238 | NP_037370 | EAGLILGVSPSAGKAKIRTAHRR |
| | | | | VMILNHPDKGGSPYVAAKINEAK |
| | | | | DLLETTTKH |
| DNAJC16 | 34 | NM_001287811 | NP_001274740 | DPYRVLGVSRTASQADIKKAYKK |
| | | | | LAREWHPDKNKDPGAEDKFIQIS |
| | | | | KAYEILSNEEKRSNYDQYG |
| DNAJC17 | 35 | NM_018163 | NP_060633 | DLYALLGIEEKAADKEVKKAYRQ |
| | | | | KALSCHPDKNPDNPRAAELFHQL |
| | | | | SQALEVLTDAAARAAYDKVR |
| DNAJC18 | 36 | NM_152686 | NP_689899 | NYYEILGVSRDASDEELKKAYRK |
| | | | | LALKFHPDKNCAPGATDAFKAIG |
| | | | | NAFAVLSNPDKRLRYDEYG |
| DNAJC19 | 37 | NM_001190233 | NP_001177162 | EAALILGVSPTANKGKIRDAHRR |
| | | | | IMLLNHPDKGGSPYIAAKINEAK |
| | | | | DLLEGQAKK |
| DNAJC20 | 38 | NM_172002 | NP_741999 | DYFSLMDCNRSFRVDTAKLQHRY |
| | | | | QQLQRLVHPDFFSQRSQTEKDFS |
| | | | | EKHSTLVNDAYKTLLAPLSRGLY |
| | | | | LLK |
| DNAJC21 | 39 | NM_001012339 | NP_001012339 | CHYEALGVRRDASEEELKKAYRK |
| | | | | LALKWHPDKNLDNAAEAAEQFKL |
| | | | | IQAAYDVLSDPQERAWYDNHR |
| DNAJC22 | 40 | NM_001304944 | NP_001291873 | LAYQVLGLSEGATNEEIHRSYQE |
| | | | | LVKVWHPDHNLDQTEEAQRHFLE |
| | | | | IQAAYEVLSQPRKPWGSRR |
| DNAJC23 | 41 | NM_007214 | NP_009145 | NPYEVLNLDPGATVAEIKKQYRL |
| | | | | LSLKYHPDKGGDEVMFMRIAKAY |
| | | | | AALTDEESRKNWEEFG |
| DNAJC24 | 42 | NM_181706 | NP_859057 | DWYSILGADPSANISDLKQKYQK |
| | | | | LILMYHPDKQSTDVPAGTVEECV |
| | | | | QKFIEIDQAWKILGNEETKREYD |
| | | | | LQR |
| DNAJC25 | 43 | NM_001015882 | NP_001015882 | DCYEVLGVSRSAGKAEIARAYRQ |
| | | | | LARRYHPDRYRPQPGDEGPGRTP |
| | | | | QSAEEAFLLVATAYETLKDEETR |
| | | | | KDYDYML |
| DNAJC26 | 44 | NM_001318134 | NP_001305063 | SRWTPVGMADLVAPEQVKKHYRR |
| | | | | AVLAVHPDKAAGQPYEQHAKMIF |
| | | | | MELNDAWSEFENQGSRPLF |
| DNAJC27 | 45 | NM_001198559 | NP_001185488 | DSWDMLGVKPGASRDEVNKAYRK |
| | | | | LAVLLHPDKCVAPGSEDAFKAVV |
| | | | | NARTALLKNIK |
| DNAJC28 | 46 | NM_001040192 | NP_001035282 | EYYRLLNVEEGCSADEVRESFHK |
| | | | | LAKQYHPDSGSNTADSATFIRIE |
| | | | | KAYRKVLSHVIEQTNASQS |
| DNAJC29 | 47 | NM_014363 | NP_055178 | ILKEVTSWEQAWKLPESERKK1 |
| | | | | IRRLYLKWHPDKNPENHDIANEV |
| | | | | FKHLQNEINRLEKQAFLDQNADR |
| | | | | ASRRTFSTSASRFQSDKYS |
| DNAJC30 | 48 | NM_032317 | NP_115693 | ALYDLLGVPSTATQAQIKAAYYR |
| | | | | QCFLYHPDRNSGSAEAAERFTRI |
| | | | | SQAYWLGSATLRRKYDRGL |
| SV40 | 49 | NC_001669 | YP_003708382 | QLMDLLGLERSAWGNIPLMRKAY |
| Jdomain | | | | LKKCKEFHPDKGGDEEKMKKMNT |
| | | | | LYKKMEDGVKYAHQPDFG |
| Bacterial | 50 | NC_000913 | NP_414556 | KQDYYEILGVSKTAEEREIRKAY |
| J-domain | | | | KRLAMKYHPDRNQGDKEAEAKFK |
| | | | | EIKEAYEVLTDSQKRAAYDQYGH |
| | | | | A |

b. Polyglutamine-Binding Domain

The fusion protein also comprises at least one polyglutamine-binding domain. The polyglutamine-binding domain can be a single chain polypeptide, or a multimeric polypeptide joined with the i domain to form the fusion protein.

It is ideal that the polyglutamine-binding domain possesses a sufficient affinity to be able to bind polyglutamine-containing proteins when present at a pathological level within cells. Therefore, in one embodiment, the fusion protein comprises a polyglutamine-binding domain that has a K_{D} for polyglutamine repeats (for example, a thioredoxin-Q₆₂ construct) of 2 μM or less, for example, 1 μM or less, 500 nM or less, 300 μM or less, 200 nM or less when tested using an indirect assay measuring inhibition of thioredoxin-Q₆₂ aggregation (Nagai et al (2000) J. Biol. Chem., 275 (14) 10437-10442).

It has been reported that QBP1 sequence (SEQ ID NO: 57) and its derivatives also block the aggregation of other amyloidogenic proteins such as TDP-43 (Mompeán et al (2019) Archives of Biochemistry and Biophysics, 675, 108113), α-synuclein (Hervás et al (2012) PLOS Biology, 10(5), e1001335), and prion homologue (Hervás et al (2012) PLOS Biology, 10(5), e1001335) and the sequences have been considered as a promiscuous inhibitor of the critical monomeric β-conformational change. Therefore, in another embodiment, the fusion protein comprising a polyglutamine-binding domain that is selected from the group consisting of SEQ ID NOs: 51-68 (see, for example, Table 2) is used to reduce protein aggregation associated with diseases caused by protein misfolding and/or such as ALS, FTD, Parkinson's disease, Huntington's disease, Alzheimer's disease, hippocampal sclerosis, prion disease, and dementia with Lewy's bodies.

Polyglutamine-binding domains have been previously identified and characterized (see, for example, Nagai et al., ibid; Waragai et al., (1999) Hum Mol Genet 8, 977-987; Imafuku et al., (1998) Biochem Biophys Res Commun 253, 16-20). Therefore, in another embodiment, the fusion protein comprises a polyglutamine-binding domain that is selected from the group consisting of SEQ ID NOs: 51-68 (see, for example, Table 2). In one particular embodiment, the fusion protein comprises the polyglutamine-binding domain of SEQ ID NO: 57. In another embodiment, the fusion protein comprises the polyglutamine-binding domain of SEQ ID NO: 62. In another embodiment, the fusion protein comprises the polyglutamine-binding domain of SEQ ID NO: 68.

In another embodiment, the fusion protein also contemplates the use of the polyglutamine-binding protein that is chemically conjugated to the J domain. The polyglutamine-binding domain can be conjugated directly to the J domain. Alternatively, it can be conjugated to the J domain by a linker. For example, there are a large number of chemical cross-linking agents that are known to those skilled in the art and useful for cross-linking the polyglutamine-binding domain to the J domain, or a targeting domain to a fusion protein comprising the polyglutamine-binding domain and J domain. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl)cyclohexane-1-carboxylate (SMCC), m-Maleimidobenzoyl-N-hydroxysuccinimide ester (MBS); N-succinimidyl(4-iodoacetyl)aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate. 2 HCl (Forbes-Cori Disease) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al., (1990) Bioconj. Chem. 1:2-12, incorporated by reference herein.

TABLE 2
Examples of Polyglutamine-binding Domains

| | SEQ | | |
|---|---|---|---|
| | ID | | |
| Name | NO: | Length | Sequence |
| PQBP1 | 51 | 265 | MPLPVALQTRLAKRGILKHLEPEPEEEIIAEDYDDDPVD |
| | | | YEATRLEGLPPSWYKVFDPSCGLPYYWNADTDLVSWLSP |
| | | | HDPNSVVTKSAKKLRSSNADAEEKLDRSHDKSDRGHDKS |
| | | | DRSHEKLDRGHDKSDRGHDKSDRDRERGYDKVDRERERD |
| | | | RERDRDRGYDKADREEGKERRHHRREELAPYPKSKKAVS |
| | | | RKDEELDPMDPSSYSDAPRGTWSTGLPKRNEAKTGADTT |
| | | | AAGPLFQQRPYPSPGAVLRANAEASRTKQQD |
| PQBP-2 | 52 | 60 | RPDQERLLLRGWVPRWPHQPAAAEAAPDRVPPELTLTLQ |
| | | | YSRNTERCGIHMAIHHCQPDN |
| PQBP-3 | 53 | 46 | RRRWWTRASWPRRRRRRGTGCCSGSPTAARPPSPWRKTR |
| | | | KGTMSLT |
| PQBP-4 | 54 | 24 | RRGTRRLKMNRLQPFITHGYLCHE |
| PQBP-5 | 55 | 41 | RRRKSRKENQVMQKVRRVQMMKKPGLKRSGSNADSSSRR |
| | | | KK |
| VCP | 56 | 35 | RFPSGNQGGAGPSQGSGSGTGGSVYTEDNDDDLYG |
| QBP1 | 57 | 11 | SNWKWWPGIFD |
| QBP2 | 58 | 11 | HWWRSWYSDSV |
| QBP3 | 59 | 11 | HEWHWWHQEAA |
| QBP4 | 60 | 11 | WGLEHFAGNKR |
| QBP5 | 61 | 11 | WWRWNWATPVD |
| QBP6 | 62 | 11 | WHNYFHWWQDT |
| MW1-LC | 63 | 116 | QLVLTQSSSASFSLGASAKLTCTLSSQHSTYTIEWYQQQ |
| | | | PLKPPKYVMELKKDGSHSTGDGIPDRFSGSSSGADRYLS |
| | | | ISNIQPEDEAIYICGVGDTIKEQFVYVFGGGTKVTVLG |
| MW1-HC | 64 | 118 | QVQLQESGGGLVQPGGSLKLSCAASGFTFRDYYMYWVRQ |
| | | | TPEKRLEWVAFISNGGGSTYYPDTVKGRFTISRDNAKNT |
| | | | LYLQMSRLKSEDTAMYYCARGRGYVWFAYWGQGTTVTVS |
| | | | S |
| 1C2-LC | 65 | 116 | QLVLTQSSSASFSLGASAKLTCTLSRQHSTYTIEWYQQQ |
| | | | PLKPPKFVMELKKDGSHSTGDGIPDRFSGSSSGAHRYLS |
| | | | ISNIQPEDEAIYICGVGDTIKEQFVYVFGGGTKVTVLG |
| PolyQ(25) | 66 | 25 | QQQQQQQQQQQQQQQQQQQQQQQQQ |
| MW7 | 67 | 243 | QVKLQESGGGLVQPGGSMKLSCAASGFTFSDAWMDWVRQ |
| | | | SPEKGLSGVAEIRSKANNHATYYAESVKGRFTISRDDSK |
| | | | SSVYLQMNSLRAEDTGIYYCIYAGFAYWGQGTTVTVSSG |
| | | | GGGSGGGGSGGGGSDIELTQSPSSLAMSVGQKVTMSCKS |
| | | | SQSLLNSSNQKNYLAWYQQKPGQSPKLLVYFASTRESGV |
| | | | PDRFIGSGSGTDFTLTISSVQAEDLADYFCQQHYSTPWT |
| | | | FGGGTKLEI |
| Happ1 | 68 | 118 | MQSVLTQPPSASGTPGQRVTISCSGSSSNIGSNYVYWYQ |
| | | | QLPGTAPKLLIYRNNQRPSGVPDRFSGSKSGTSASLAIS |
| | | | GLRPEDEADYYCAAWDDSLCVALVFGGGTNGGGGVDGTA |
| | | | G |

c. Optional Linker

The fusion proteins described herein can optionally contain one or more linkers. Linkers can be peptidic or non-peptidic. The purpose of the linker is to provide, among other things, an adequate distance between functional domains within the protein (e.g., between the i domain and polyglutamine-binding domain, between tandem arrangements of polyglutamine-binding domains, between either the i domain and polyglutamine-binding domain and an optional targeting reagent, or between either the i domain and polyglutamine-binding domain and an optional detection domain or epitope) for optimal function of each of the domains. Clearly, a linker preferably does not interfere with the respective functions of the i domain, the target protein binding domain of a fusion protein according to the invention. A linker, if present in a fusion protein of the invention, is selected to attenuate the cytotoxicity caused by target proteins (polyQ proteins), and it may be omitted if direct attachment achieves a desired effect. Linkers present in a fusion protein of the invention may comprise one or more amino acids encoded by a nucleotide sequence present on a segment of nucleic acid in or around a cloning site of an expression vector into which is inserted in frame a nucleic acid segment encoding a protein domain or an entire fusion protein as described herein. In one embodiment, the peptide linker is between 1 amino acid and 20 amino acids in length. In another embodiment, the peptide linker is between 2 amino acids and 15 amino acids in length. In still another embodiment, the peptide linker is between 2 amino acids and 10 amino acids in length.

Selecting one or more polypeptide linkers to produce a fusion protein according to the invention is within the knowledge and skill of practitioners in the art. See, for example, Arai et al., Protein Eng., 14(8): 529-532 (2001); Crasto et al., Protein Eng., 13(5): 309-314 (2000); George et al., Protein Eng., 15(11): 871-879 (2003); Robinson et al., Proc. Natl. Acad. Sci. USA, 95: 5929-5934 (1998), each of which is incorporated herein by reference in its entirety.

Examples of linkers of two or more amino acids that may be used in preparing a fusion protein according to the invention, include, by are not limited to, those provided below in Table 3.

TABLE 3
Linker Sequences

| SEQ ID | | |
|---|---|---|
| NO: | Length | Sequence |
| 69 | 2 | SR |
| 70 | 4 | GTGS |
| 71 | 5 | GLESR |
| 72 | 4 | GGSG |
| 73 | 4 | GGGS |
| 74 | 5 | DIAAA |
| 75 | 9 | DIAAALESR |
| 76 | 15 | GGGGSGGGGSGGGGS |
| 77 | 11 | AEAAAKEAAAK |
| 78 | 15 | SGGGSGGGGSGGGGS |
| 79 | 25 | DIGGGGSGGGGSGGGGSGGGGSAAA |
| 80 | 5 | GGGGS |
| 81 | 5 | EAAAK |
| 82 | 10 | GGGGSGGGGS |
| 83 | 10 | EAAAKEAAAK |
| 84 | 2 | LE |

d. Targeting Reagents

The fusion proteins disclosed herein can further comprise a targeting moiety. As used herein, the terms “targeting moiety” and “targeting reagent” are used interchangeably and refer to a substance associated with the fusion protein that enhances binding, transport, accumulation, residence time, bioavailability, or modifies biological activity or therapeutic effect of the fusion protein in a cell or in the body of a subject. A targeting moiety can have functionality at the tissue, cellular, and/or subcellular level. The targeting moiety can direct localization of the fusion protein to a particular cell, tissue or organ, for example, upon administration of the fusion protein into a subject. In one embodiment, the targeting moiety is located at the N-terminus of the fusion protein. In another embodiment, the targeting moiety is located at the C-terminus of the fusion protein. In still another embodiment, the targeting moiety is located internally. In another embodiment, the targeting moiety is attached to the fusion protein via chemical conjugation.

The targeting moiety can include, but is not limited to, an organic or inorganic molecule, a peptide, a peptide mimetic, a protein, an antibody or fragment thereof, a growth factor, an enzyme, a lectin, an antigen or immunogen, viruses or component thereof, a viral vector, a receptors, a receptor ligand, a toxins, a polynucleotide, an oligonucleotide or aptamer, a nucleotide, a carbohydrate, a sugar, a lipid, a glycolipid, a nucleoprotein, a glycoprotein, a lipoprotein, a steroid, a hormone, a growth factor, a chemoattractant, a cytokine, a chemokine, a drug, or a small molecule, among others.

In an exemplary embodiment of the present invention, the targeting moiety enhances binding, transport, accumulation, residence time, bioavailability, or modifies biological activity of the modifies biological activity or therapeutic effect of the platform, or its associated ligand and/or active agent in the target cell or tissue, for example, neuronal cells, the central nervous system, and/or the peripheral nervous system. Thus, the targeting moiety can have specificity for cellular receptors associated with the central nervous system, or is otherwise associated with enhanced delivery to the CNS via the blood-brain barrier (BBB). Consequently, a ligand, as described above, can be both a ligand and a targeting moiety.

In some embodiments, the targeting moiety can be a cell-penetrating peptide, for example, as described in U.S. Pat. No. 10,111,965, which is incorporated by reference in its entirety. In another embodiment, the targeting moiety can be an antibody or an antigen-binding fragment or single-chain derivative thereof, for example, as described in U.S. Ser. No. 16/131,591, which is incorporated herein by reference in its entirety.

The targeting moiety can be coupled to the platform for targeted cellular delivery by being directly or indirectly bound to the core. For example, in embodiments where the core comprises a nanoparticle, conjugation of the targeting moiety to the nanoparticle can utilize similar functional groups that are employed to tether PEG to the nanoparticle. Thus, the targeting moiety can be directly bound to the nanoparticle through functionalization of the targeting moiety. Alternatively, the targeting moiety can be indirectly bound to the nanoparticle through conjugation of the targeting moiety to a functionalized PEG, as discussed above. A targeting moiety can be attached to core by way of covalent, non-covalent, or electrostatic interactions. In one embodiment, the targeting moiety is a peptide. In a particular embodiment, the targeting moiety is a peptide that is covalently attached to the N-terminus of the fusion protein.

e. Epitopes

In certain embodiments, the fusion protein of the present invention contains an optional epitope or tag, which can impart additional properties to the fusion protein. As used herein, the terms “epitope” and “tag” are used interchangeably to refer to an amino acid sequence, typically 300 amino acids or less in length, which is typically attached to the N-terminal or C-terminal end of the fusion protein. In one embodiment, the fusion protein of the present invention further comprises an epitope which is used to facilitate purification. Examples of such epitopes useful for purification include the human IgG1 Fc sequence (SEQ ID NO: 162), the FLAG epitope (DYKDDDDK, SEQ ID NO: 163), His6 epitope (SEQ ID NO: 164), c-myc (SEQ ID NO: 165), HA (SEQ ID NO: 166), V5 epitope (SEQ ID NO: 167), or glutathione-s-transferase (SEQ ID NO:168). In another embodiment, the fusion protein of the present invention further comprises an epitope which is used to increase the half-life of the fusion protein when administered into a subject, for example a human. Examples of such epitopes useful for increasing half-life include the human Fc sequence. Therefore, in one particular embodiment, the fusion protein comprises, in addition to a J domain and polyglutamine-binding domain, a human Fc epitope. The epitope is positioned at the C-terminal end of the fusion protein.

f. Cell-Penetrating Peptides

In still other embodiments, the fusion protein described herein can further comprise a cell-penetrating peptide. Cell-penetrating peptides are known to carry a conjugated cargo, whether a small molecule, peptide, protein or nucleic acid, into cells. Non-limiting examples of cell-penetrating peptides in a fusion protein of the invention include, but are not limited to, a polycationic peptide, e.g., an HIV TAT peptide49-57, polyarginines, and penetratin pAntan(43-58), amphipathic peptide, e.g., pep-1, a hydrophobic peptide, e.g., a C405Y, and the like. See Table 4 below.

TABLE 4
Examples of Cel1-Penetrating Peptides

| SEQID NO: | SEQUENCE |
|---|---|
| 85 | RKKRRQRRR |
| 86 | RQIKWFQNRRMKWKK |
| 87 | KETWWETWWTEWSQPKKKRKV |
| 88 | CSIPPEVKFNKPFVYLI |

Therefore, in one embodiment, the fusion protein further comprises a cell-penetrating peptide and a fusion protein, wherein the cell-penetrating peptide is selected from the group consisting of SEQ ID NOs: 85-88, and the fusion protein is selected from the group consisting of SEQ ID NOs: 89-158. In another embodiment, the fusion protein comprises the signal sequence of SEQ ID NO: 85, and the fusion protein selected from the group consisting of SEQ ID NOs: 89-158. In another embodiment, the fusion protein comprises the cell-penetrating peptide of SEQ ID NO: 86, and the fusion protein selected from the group consisting of SEQ ID NOs: 89-158. In still another embodiment, the fusion protein comprises the cell-penetrating peptide of SEQ ID NO: 87, and the fusion protein selected from the group consisting of SEQ ID NOs: 89-158. In yet another embodiment, the fusion protein comprises the cell-penetrating peptide of SEQ ID NO: 88, and the fusion protein selected from the group consisting of SEQ ID NOs: 89-158. Cells expressing the fusion protein constructs with the cell-penetrating peptide can be administered to a subject, for example a human subject (e.g., a patient having or at risk of suffering from a polyglutamine repeat disorder). The fusion protein is secreted from the cells, which help reduce polyglutamine repeat-containing protein aggregation and/or associated cytotoxicity.

In another embodiment, the fusion protein further comprises a signal sequence, which is positioned on the N-terminus. The signal sequence can be selected from the group consisting of SEQ ID NOs:159-161.

g. Arrangement of J Domain and Polyglutamine Binding Domain

The fusion proteins described herein can be arranged in a multitude of ways. In one embodiment, the polyglutamine binding domain is attached to the C-terminal side of the J domain. In another embodiment, the polyglutamine binding domain is attached to the N-terminal side of the J domain. The polyglutamine domain and the J domain, in either configuration, can optionally be separated via a linker as described above.

In some embodiments, the J domain can be attached to a plurality of polyglutamine binding domains, for example, two polyglutamine-binding domains, three polyglutamine-binding domains, four polyglutamine-binding domains or more. The polyglutamine-binding domains can be attached to the N-terminal side of the J domain. Alternatively, the polyglutamine-binding domains can be attached to the C-terminal side of the J domain. In still another embodiment, the polyglutamine-binding domains can be attached on the N-terminal and C-terminal sides of the J domain. Each of the plurality of polyglutamine-binding domains can be the same polyglutamine-binding domain. In another embodiment, each of the plurality of polyglutamine-binding domains in the fusion protein can be different polyglutamine-binding domains (i.e., different sequences).

In some embodiments, the fusion proteins can comprise a structure selected from the following group:
a. DNAJ-X-Q,
b. DNAJ-X-Q-X-Q,
c. DNAJ-X-Q-X-Q-X-Q,
d. Q-X-DNAJ,
e. Q-X-Q-X-DNAJ,
f. Q-X-Q-X-Q-X-DNAJ,
g. Q-X-DNAJ-X-Q,
h. Q-X-DNAJ-X-Q-X-Q,
i. DNAJ-X-DNAJ-X-Q,
j. Q-X-Q-X-DNAJ-X-Q,
k. DNAJ-X-Q-X-DNAJ-X-Q,
l. Q-X-Q-X-DNAJ-X-Q-X-Q-X-Q,
m. Q-X-Q-X-Q-X-DNAJ-X-Q,
n. Q-X-Q-X-Q-X-DNAJ-X-Q-X-Q,
o. Q-X-Q-X-Q-X-DNAJ-X-Q-X-Q-X-Q,
p. DnaJ-X-DnaJ-X-Q-X-Q,
q. Q-X-DnaJ-X-DnaJ,
r. Q-X-Q-X-DnaJ-X-DnaJ, and
s. Q-X-DnaJ-X-DnaJ-X-Q
   wherein,
   Q is a polyglutamine-binding domain,
   DNAJ is a J domain of a J protein, and
   X is an optional linker.

In one embodiment, the fusion protein comprises the J domain selected from the group consisting of SEQ ID NOs: 5, 6, 10, 24, and 31. In one particular embodiment, the fusion protein comprises the J domain of SEQ ID NO: 5.

In another embodiment, the polyglutamine-binding domain is selected from the group consisting of SEQ ID NOs: 57, 62 and 68. In one particular embodiment, the polyglutamine-binding domain is SEQ ID NO:57.

In still another embodiment, the fusion protein comprises the J domain of SEQ ID NO: 5, and the polyglutamine-binding domain of SEQ ID NO:57. In one embodiment, the fusion protein comprises a sequence selected from the group consisting of SEQ ID NOs: 90-104, 122-145, 147, and 152-158.

In another embodiment, the fusion protein comprises the J domain of SEQ ID NO: 5, and at least two copies of the polyglutamine-binding domain of SEQ ID NO:57. In a particular embodiment, the fusion protein comprises a sequence selected from the group consisting of SEQ ID NOs: 92-95, 103-104, and 122-143.

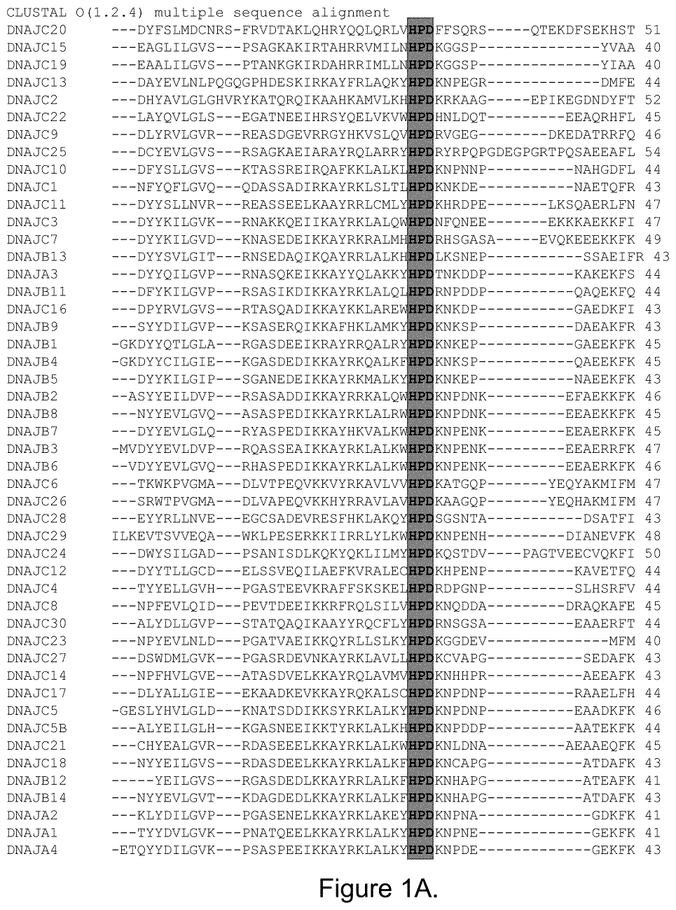

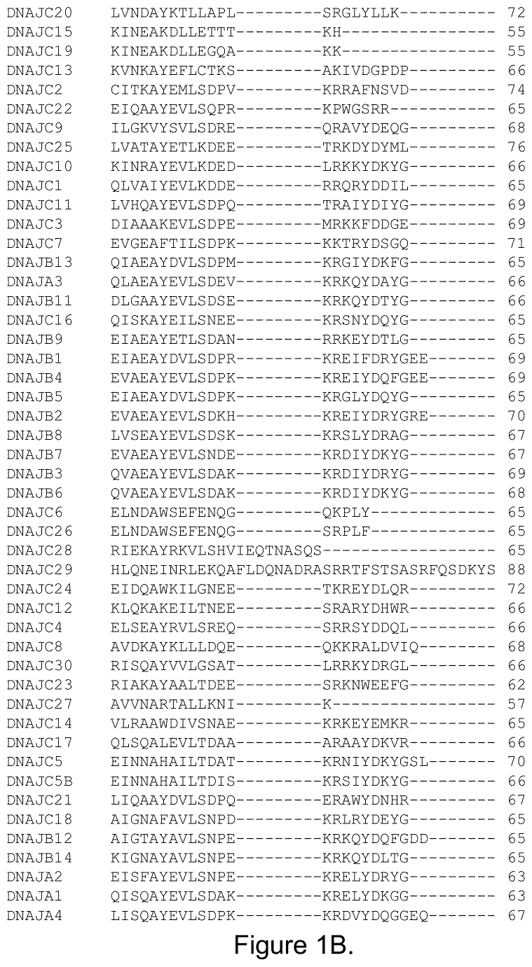

Non-limiting examples of fusion protein constructs comprising a J domain and polyglutamine-binding domain are depicted schematically in FIG. 1, and also shown below in Table 5. In another embodiment, the specific fusion protein construct is selected from the group consisting of SEQ ID NO: 89-157.

TABLE 5
Fusion Protein Constructs

| | | SEQ | | |
|---|---|---|---|---|
| Construct | | ID | | |
| No | Construct Name | NO: | Length | Sequence |
| 1 | JB1 | 89 | 92 | MGKDYYQTLGLARGASDEEIKRAYRRQALRYH |
| | | | | PDKNKEPGAEEKFKEIAEAYDVLSDPRKREIF |
| | | | | DRYGEEGLKGSDIAAALESRDYKDDDDK |
| 2 | JBI-QBP1 | 90 | 114 | MGKDYYQTLGLARGASDEEIKRAYRRQALRYH |
| | | | | PDKNKEPGAEEKFKEIAEAYDVLSDPRKREIF |
| | | | | DRYGEEGLKGSDIGGGGSGGGGSGGGGSGGGG |
| | | | | SAAASNWKWWPGIFDGLE |
| 3 | QBP1-JB1 | 91 | 114 | MGKPIPNPLLGLDSTGTGSSNWKWWPGIFDEF |
| | | | | MGKDYYQTLGLARGASDEEIKRAYRRQALRYH |
| | | | | PDKNKEPGAEEKFKEIAEAYDVLSDPRKREIF |
| | | | | DRYGEEGLKGSDIAAALE |
| 4 | JB1-2XQBP1 | 92 | 125 | MGKDYYQTLGLARGASDEEIKRAYRRQALRYH |
| | | | | PDKNKEPGAEEKFKEIAEAYDVLSDPRKREIF |
| | | | | DRYGEEGLKGSDIGGGGSGGGGSGGGGSGGGG |
| | | | | SAAASNWKWWPGIFDGLESNWKWWPGIFD |
| 5 | JB1-3XQBP1 | 93 | 131 | MGKDYYQTLGLARGASDEEIKRAYRRQALRYH |
| | | | | PDKNKEPGAEEKFKEIAEAYDVLSDPRKREIF |
| | | | | DRYGEEGLKGSGGGGSGGGGSGGGGSGGGGSG |
| | | | | SSNWKWWPGIAAASNWKWWPGILESNWKWWPG |
| | | | | IFD |
| 6 | QBP1-JB1-QBP1 | 94 | 146 | MGKPIPNPLLGLDSTGTGSSNWKWWPGIFDEF |
| | | | | MGKDYYQTLGLARGASDEEIKRAYRRQALRYH |
| | | | | PDKNKEPGAEEKFKEIAEAYDVLSDPRKREIF |
| | | | | DRYGEEGLKGSDIGGGGSGGGGSGGGGSGGGG |
| | | | | SAAASNWKWWPGIFDGLE |
| 7 | QBP1-JB1- | 95 | 157 | MGKPIPNPLLGLDSTGTGSSNWKWWPGIFDEF |
| | 2XQBP1 | | | MGKDYYQTLGLARGASDEEIKRAYRRQALRYH |
| | | | | PDKNKEPGAEEKFKEIAEAYDVLSDPRKREIF |
| | | | | DRYGEEGLKGSDIGGGGSGGGGSGGGGSGGGG |
| | | | | SAAASNWKWWPGIFDGLESNWKWWPGIFD |
| 8 | JB1(P33Q)- | 96 | 125 | MGKDYYQTLGLARGASDEEIKRAYRRQALRYH |
| | 2XQBP1 | | | QDKNKEPGAEEKFKEIAEAYDVLSDPRKREIF |
| | | | | DRYGEEGLKGSDIGGGGSGGGGSGGGGSGGGG |
| | | | | SAAASNWKWWPGIFDGLESNWKWWPGIFD |
| 9 | JB1(20-31)- | 97 | 69 | MGTGSEFIKRAYRRQALRYDIGGGGSGGGGSG |
| | 2XQBP1 | | | GGGSGGGGSAAASNWKWWPGIFDGLESNWKWW |
| | | | | PGIFD |
| 10 | JB1(19-43)- | 98 | 82 | MGTGSEFEIKRAYRRQALRYHPDKNKEPGAEE |
| | 2XQBP1 | | | DIGGGGSGGGGSGGGGSGGGGSAAASNWKWWP |
| | | | | GIFDGLESNWKWWPGIFD |
| 11 | JB1(17-75)- | 99 | 116 | MGTGSEFDEEIKRAYRRQALRYHPDKNKEPGA |
| | 2XQBP1 | | | EEKFKEIAEAYDVLSDPRKREIFDRYGEEGLK |
| | | | | GSDIGGGGSGGGGSGGGGSGGGGSAAASNWKW |
| | | | | WPGIFDGLESNWKWWPGIFD |
| 12 | JB1(1-57)- | 100 | 107 | MGKDYYQTLGLARGASDEEIKRAYRRQALRYH |
| | 2XQBP1 | | | PDKNKEPGAEEKFKEIAEAYDVLSDDIGGGGS |
| | | | | GGGGSGGGGSGGGGSAAASNWKWWPGIFDGLE |
| | | | | SNWKWWPGIFD |
| 13 | JB1(1-67)- | 101 | 117 | MGKDYYQTLGLARGASDEEIKRAYRRQALRYH |
| | 2XQBP1 | | | PDKNKEPGAEEKFKEIAEAYDVLSDPRKREIF |
| | | | | DRYDIGGGGSGGGGSGGGGSGGGGSAAASNWK |
| | | | | WWPGIFDGLESNWKWWPGIFD |
| 14 | JB1(1-108)- | 102 | 158 | MGKDYYQTLGLARGASDEEIKRAYRRQALRYH |
| | 2XQBP1 | | | PDKNKEPGAEEKFKEIAEAYDVLSDPRKREIF |
| | | | | DRYGEEGLKGSGPSGGSGGGANGTSFSYTFHG |
| | | | | DPHAMFAEFFGGDIGGGGSGGGGSGGGGSGGG |
| | | | | GSAAASNWKWWPGIFDGLESNWKWWPGIFD |
| 15 | JB1-2XQBP1 (no | 103 | 105 | MGKDYYQTLGLARGASDEEIKRAYRRQALRYH |
| | flexible linker) | | | PDKNKEPGAEEKFKEIAEAYDVLSDPRKREIF |
| | | | | DRYGEEGLKGSDIAAASNWKWWPGIFDGLESN |
| | | | | WKWWPGIFD |
| 16 | JB1-2XQBP1 | 104 | 110 | MGKDYYQTLGLARGASDEEIKRAYRRQALRYH |
| | (rigid linker) | | | PDKNKEPGAEEKFKEIAEAYDVLSDPRKREIF |
| | | | | DRYGEEGLKGSDIEAAAKAAASNWKWWPGIFD |
| | | | | GLESNWKWWPGIFD |
| 17 | JB2-2XQBP1 | 105 | 121 | MASYYEILDVPRSASADDIKKAYRRKALQWHP |
| | | | | DKNPDNKEFAEKKFKEVAEAYEVLSDKHKREI |
| | | | | YDRYGREDIGGGGSGGGGSGGGGSGGGGSAAA |
| | | | | SNWKWWPGIFDGLESNWKWWPGIFD |
| 18 | JB6-2XQBP1 | 106 | 119 | MVDYYEVLGVQRHASPEDIKKAYRKLALKWHP |
| | | | | DKNPENKEEAERKFKQVAEAYEVLSDAKKRDI |
| | | | | YDKYGDIGGGGSGGGGSGGGGSGGGGSAAASN |
| | | | | WKWWPGIFDGLESNWKWWPGIFD |
| 19 | JC6-2XQBP1 | 107 | 116 | MTKWKPVGMADLVTPEQVKKVYRKAVLWHPD |
| | | | | KATGQPYEQYAKMIFMELNDAWSEFENQGQKP |
| | | | | LYDIGGGGSGGGGSGGGGSGGGGSAAASNWKW |
| | | | | WPGIFDGLESNWKWWPGIFD |
| 20 | JB1-2XQBP2 | 108 | 125 | MGKDYYQTLGLARGASDEEIKRAYRRQALRYH |
| | | | | PDKNKEPGAEEKFKEIAEAYDVLSDPRKREIF |
| | | | | DRYGEEGLKGSDIGGGGSGGGGSGGGGSGGGG |
| | | | | SAAAHWWRSWYSDSVGLEHWWRSWYSDSV |
| 21 | JB1-scFv(MW1) | 109 | 349 | MGKDYYQTLGLARGASDEEIKRAYRRQALRYH |
| | | | | PDKNKEPGAEEKFKEIAEAYDVLSDPRKREIF |
| | | | | DRYGEEGLKGSDIGGGGSGGGGSGGGGSGGGG |
| | | | | SAAAQVQLQESGGGLVQPGGSLKLSCAASGFT |
| | | | | FRDYYMYWVRQTPEKRLEWVAFISNGGGSTYY |
| | | | | PDTVKGRFTISRDNAKNTLYLQMSRLKSEDTA |
| | | | | MYYCARGRGYVWFAYWGQGTTVTVSSGGGGSG |
| | | | | GGGSGGGGSQLVLTQSSSASFSLGASAKLTCT |
| | | | | LSSQHSTYTIEWYQQQPLKPPKYVMELKKDGS |
| | | | | HSTGDGIPDRFSGSSSGADRYLSISNIQPEDE |
| | | | | AIYICGVGDTIKEQFVYVFGGGTKVTVLG |
| 22 | JB1-polyQ(25) | 110 | 125 | MGKDYYQTLGLARGASDEEIKRAYRRQALRYH |
| | | | | PDKNKEPGAEEKFKEIAEAYDVLSDPRKREIF |
| | | | | DRYGEEGLKGSDIGGGGSGGGGSGGGGSGGGG |
| | | | | SAAAQQQQQQQQQQQQQQQQQQQQQQQQQ |
| 23 | JB2-2XQBP2 | 111 | 121 | MASYYEILDVPRSASADDIKKAYRRKALQWHP |
| | | | | DKNPDNKEFAEKKFKEVAEAYEVLSDKHKREI |
| | | | | YDRYGREDIGGGGSGGGGSGGGGSGGGGSAAA |
| | | | | HWWRSWYSDSVGLEHWWRSWYSDSV |
| 24 | JB6-2XQBP2 | 112 | 119 | MVDYYEVLGVQRHASPEDIKKAYRKLALKWHP |
| | | | | DKNPENKEEAERKFKQVAEAYEVLSDAKKRDI |
| | | | | YDKYGDIGGGGSGGGGSGGGGSGGGGSAAAHW |
| | | | | WRSWYSDSVGLEHWWRSWYSDSV |
| 25 | JC6-2XQBP2 | 113 | 116 | MTKWKPVGMADLVTPEQVKKVYRKAVLWHPD |
| | | | | KATGQPYEQYAKMIFMELNDAWSEFENQGQKP |
| | | | | LYDIGGGGSGGGGSGGGGSGGGGSAAAHWWRS |
| | | | | WYSDSVGLEHWWRSWYSDSV |
| 26 | JB2-scFv(MW1) | 114 | 345 | MASYYEILDVPRSASADDIKKAYRRKALQWHP |
| | | | | DKNPDNKEFAEKKFKEVAEAYEVLSDKHKREI |
| | | | | YDRYGREDIGGGGSGGGGSGGGGSGGGGSAAA |
| | | | | QVQLQESGGGLVQPGGSLKLSCAASGFTFRDY |
| | | | | YMYWVRQTPEKRLEWVAFISNGGGSTYYPDTV |
| | | | | KGRFTISRDNAKNTLYLQMSRLKSEDTAMYYC |
| | | | | ARGRGYVWFAYWGQGTTVTVSSGGGGSGGGGS |
| | | | | GGGGSQLVLTQSSSASFSLGASAKLTCTLSSQ |
| | | | | HSTYTIEWYQQQPLKPPKYVMELKKDGSHSTG |
| | | | | DGIPDRFSGSSSGADRYLSISNIQPEDEAIYI |
| | | | | CGVGDTIKEQFVYVFGGGTKVTVLG |
| 27 | JB6-scFv(MW1) | 115 | 343 | MVDYYEVLGVQRHASPEDIKKAYRKLALKWHP |
| | | | | DKNPENKEEAERKFKQVAEAYEVLSDAKKRDI |
| | | | | YDKYGDIGGGGSGGGGSGGGGSGGGGSAAAQV |
| | | | | QLQESGGGLVQPGGSLKLSCAASGFTFRDYYM |
| | | | | YWVRQTPEKRLEWVAFISNGGGSTYYPDTVKG |
| | | | | RFTISRDNAKNTLYLQMSRLKSEDTAMYYCAR |
| | | | | GRGYVWFAYWGQGTTVTVSSGGGGSGGGGSGG |
| | | | | GGSQLVLTQSSSASFSLGASAKLTCTLSSQHS |
| | | | | TYTIEWYQQQPLKPPKYVMELKKDGSHSTGDG |
| | | | | IPDRFSGSSSGADRYLSISNIQPEDEAIYICG |
| | | | | VGDTIKEQFVYVFGGGTKVTVLG |
| 28 | JC6-scFv(MW1) | 116 | 340 | MTKWKPVGMADLVTPEQVKKVYRKAVLWHPD |
| | | | | KATGQPYEQYAKMIFMELNDAWSEFENQGQKP |
| | | | | LYDIGGGGSGGGGSGGGGSGGGGSAAAQVQLQ |
| | | | | ESGGGLVQPGGSLKLSCAASGFTFRDYYMYWV |
| | | | | RQTPEKRLEWVAFISNGGGSTYYPDTVKGRFT |
| | | | | ISRDNAKNTLYLQMSRLKSEDTAMYYCARGRG |
| | | | | YVWFAYWGQGTTVTVSSGGGGSGGGGSGGGGS |
| | | | | QLVLTQSSSASFSLGASAKLTCTLSSQHSTYT |
| | | | | IEWYQQQPLKPPKYVMELKKDGSHSTGDGIPD |
| | | | | RFSGSSSGADRYLSISNIQPEDEAIYICGVGD |
| | | | | TIKEQFVYVFGGGTKVTVLG |
| 29 | JB2-polyQ(25) | 117 | 121 | MASYYEILDVPRSASADDIKKAYRRKALQWHP |
| | | | | DKNPDNKEFAEKKFKEVAEAYEVLSDKHKREI |
| | | | | YDRYGREDIGGGGSGGGGSGGGGSGGGGSAAA |
| | | | | QQQQQQQQQQQQQQQQQQQQQQQQQ |
| 30 | JB6-polyQ(25) | 118 | 119 | MVDYYEVLGVQRHASPEDIKKAYRKLALKWHP |
| | | | | DKNPENKEEAERKFKQVAEAYEVLSDAKKRDI |
| | | | | YDKYGDIGGGGSGGGGSGGGGSGGGGSAAAQQ |
| | | | | QQQQQQQQQQQQQQQQQQQQQQQ |
| 31 | JC6-polyQ(25) | 119 | 116 | MTKWKPVGMADLVTPEQVKKVYRKAVLWHPD |
| | | | | KATGQPYEQYAKMIFMELNDAWSEFENQGQKP |
| | | | | LYDIGGGGSGGGGSGGGGSGGGGSAAAQQQQQ |
| | | | | QQQQQQQQQQQQQQQQQQQQ |
| 32 | JB1-scFv(MW7) | 120 | 339 | MASYYEILDVPRSASADDIKKAYRRKALQWHPDK |
| | | | | NPDNKEFAEKKFKEVAEAYEVLSDKHKREIYDRY |
| | | | | GREDIGGGGSGGGGSGGGGSGGGGSAAAQVKLQE |
| | | | | SGGGLVQPGGSMKLSCAASGFTFSDAWMDWVRQS |
| | | | | PEKGLSGVAEIRSKANNHATYYAESVKGRFTISR |
| | | | | DDSKSSVYLQMNSLRAEDTGIYYCIYAGFAYWGQ |
| | | | | GTTVTVSSGGGGSGGGGSGGGGSDIELTQSPSSL |
| | | | | AMSVGQKVTMSCKSSQSLLNSSNQKNYLAWYQQK |
| | | | | PGQSPKLLVYFASTRESGVPDRFIGSGSGTDFTL |
| | | | | TISSVQAEDLADYFCQQHYSTPWTFGGGTKLEI |
| 33 | JBI-Happi | 121 | 214 | MASYYEILDVPRSASADDIKKAYRRKALQWHPDK |
| | | | | NPDNKEFAEKKFKEVAEAYEVLSDKHKREIYDRY |
| | | | | GREDIGGGGSGGGGSGGGGSGGGGSAAAMQSVLT |
| | | | | QPPSASGTPGQRVTISCSGSSSNIGSNYVYWYQQ |
| | | | | LPGTAPKLLIYRNNQRPSGVPDRFSGSKSGTSAS |
| | | | | LAISGLRPEDEADYYCAAWDDSLCVALVFGGGTN |
| | | | | GGGGVDGTAG |
| 34 | JB1-QBP1-QBP1 | 122 | 139 | MGKDYYQTLGLARGASDEEIKRAYRRQALRYHPD |
| | (1) | | | KNKEPGAEEKFKEIAEAYDVLSDPRKREIFDRYG |
| | | | | EEGLKGSGGGGSGGGGSGGGGSGGGGSAAASNWK |
| | | | | WWPGIFDLEGDYKDDDDKGSRAAASNWKWWPGIF |
| | | | | DLE |
| 35 | JB1-QBP1-QBP1 | 123 | 104 | MGKDYYQTLGLARGASDEEIKRAYRRQALRYHPD |
| | (2) | | | KNKEPGAEEKFKEIAEAYDVLSDPRKREIFDRYG |
| | | | | EEGLKGSSNWKWWPGIFDEAAAKSNWKWWPGIFD |
| | | | | LE |
| 36 | JB1-QBP1-QBP1 | 124 | 104 | MGKDYYQTLGLARGASDEEIKRAYRRQALRYHPD |
| | (3) | | | KNKEPGAEEKFKEIAEAYDVLSDPRKREIFDRYG |
| | | | | EEGLKGSSNWKWWPGIFDGGGGSSNWKWWPGIFD |
| | | | | LE |
| 37 | JB1-QBP1-QBP1 | 125 | 109 | MGKDYYQTLGLARGASDEEIKRAYRRQALRYHPD |
| | (4) | | | KNKEPGAEEKFKEIAEAYDVLSDPRKREIFDRYG |
| | | | | EEGLKGSGGGGSSNWKWWPGIFDEAAAKSNWKWW |
| | | | | PGIFDLE |
| 38 | JB1-QBP1-QBP1 | 126 | 109 | MGKDYYQTLGLARGASDEEIKRAYRRQALRYHPD |
| | (5) | | | KNKEPGAEEKFKEIAEAYDVLSDPRKREIFDRYG |
| | | | | EEGLKGSGGGGSSNWKWWPGIFDGGGGSSNWKWW |
| | | | | PGIFDLE |
| 39 | JB1-QBP1-QBP1 | 127 | 109 | MGKDYYQTLGLARGASDEEIKRAYRRQALRYHPD |
| | (6) | | | KNKEPGAEEKFKEIAEAYDVLSDPRKREIFDRYG |
| | | | | EEGLKGSEAAAKSNWKWWPGIFDEAAAKSNWKWW |
| | | | | PGIFDLE |
| 40 | JB1-QBP1-QBP1 | 128 | 109 | MGKDYYQTLGLARGASDEEIKRAYRRQALRYHPD |
| | (7) | | | KNKEPGAEEKFKEIAEAYDVLSDPRKREIFDRYG |
| | | | | EEGLKGSEAAAKSNWKWWPGIFDGGGGSSNWKWW |
| | | | | PGIFDLE |
| 41 | JB1-QBP1-QBP1 | 129 | 107 | MGKDYYQTLGLARGASDEEIKRAYRRQALRYHPD |
| | (8) | | | KNKEPGAEEKFKEIAEAYDVLSDPRKREIFDRYG |
| | | | | EEGLKGSEAAAKSNWKWWPGIFDEAAAKSNWKWW |
| | | | | PGIFD |
| 42 | JB1-QBP1-QBP1 | 130 | 107 | MGKDYYQTLGLARGASDEEIKRAYRRQALRYHPD |
| | (9) | | | KNKEPGAEEKFKEIAEAYDVLSDPRKREIFDRYG |
| | | | | EEGLKGSEAAAKSNWKWWPGIFDGGGGSSNWKWW |
| | | | | PGIFD |
| 43 | JB1-QBP1-QBP1 | 131 | 109 | MGKDYYQTLGLARGASDEEIKRAYRRQALRYHPD |
| | (10) | | | KNKEPGAEEKFKEIAEAYDVLSDPRKREIFDRYG |
| | | | | EEGLKGSSNWKWWPGIFDGGGGSGGGGSSNWKWW |
| | | | | PGIFDLE |
| 44 | JB1-QBP1-QBP1 | 132 | 109 | MGKDYYQTLGLARGASDEEIKRAYRRQALRYHPD |
| | (11) | | | KNKEPGAEEKFKEIAEAYDVLSDPRKREIFDRYG |
| | | | | EEGLKGSSNWKWWPGIFDEAAAKEAAAKSNWKWW |
| | | | | PGIFDLE |
| 45 | JB1-QBP1-QBP1 | 133 | 114 | MGKDYYQTLGLARGASDEEIKRAYRRQALRYHPD |
| | (12) | | | KNKEPGAEEKFKEIAEAYDVLSDPRKREIFDRYG |
| | | | | EEGLKGSGGGGSSNWKWWPGIFDGGGGSGGGGSS |
| | | | | NWKWWPGIFDLE |
| 46 | JB1-QBP1-QBP1 | 134 | 114 | MGKDYYQTLGLARGASDEEIKRAYRRQALRYHPD |
| | (13) | | | KNKEPGAEEKFKEIAEAYDVLSDPRKREIFDRYG |
| | | | | EEGLKGSGGGGSSNWKWWPGIFDEAAAKEAAAKS |
| | | | | NWKWWPGIFDLE |
| 47 | JB1-QBP1-QBP1 | 135 | 114 | MGKDYYQTLGLARGASDEEIKRAYRRQALRYHPD |
| | (14) | | | KNKEPGAEEKFKEIAEAYDVLSDPRKREIFDRYG |
| | | | | EEGLKGSEAAAKSNWKWWPGIFDGGGGSGGGGSS |
| | | | | NWKWWPGIFDLE |
| 48 | JB1-QBP1-QBP1 | 136 | 114 | MGKDYYQTLGLARGASDEEIKRAYRRQALRYHPD |
| | (15) | | | KNKEPGAEEKFKEIAEAYDVLSDPRKREIFDRYG |
| | | | | EEGLKGSEAAAKSNWKWWPGIFDEAAAKEAAAKS |
| | | | | NWKWWPGIFDLE |
| 49 | JB1-QBP1-QBP1 | 137 | 134 | MGKDYYQTLGLARGASDEEIKRAYRRQALRYHPD |
| | (16) | | | KNKEPGAEEKFKEIAEAYDVLSDPRKREIFDRYG |
| | | | | EEGLKGSGGGGSGGGGSGGGGSGGGGSAAASNWK |
| | | | | WWPGIFDLEGGGGSSRAAASNWKWWPGIFDLE |
| 50 | JB1-QBP1-QBP1 | 138 | 134 | MGKDYYQTLGLARGASDEEIKRAYRRQALRYHPD |
| | (17) | | | KNKEPGAEEKFKEIAEAYDVLSDPRKREIFDRYG |
| | | | | EEGLKGSGGGGSGGGGSGGGGSGGGGSAAASNWK |
| | | | | WWPGIFDLEEAAAKSRAAASNWKWWPGIFDLE |
| 51 | JB1-QBP1-QBP1 | 139 | 111 | MGKDYYQTLGLARGASDEEIKRAYRRQALRYHPD |
| | (18) | | | KNKEPGAEEKFKEIAEAYDVLSDPRKREIFDRYG |
| | | | | EEGLKGSEAAAKSNWKWWPGIFDLEGGGGSSNWK |
| | | | | WWPGIFDLE |
| 52 | JB1-QBP1-QBP1 | 140 | 111 | MGKDYYQTLGLARGASDEEIKRAYRRQALRYHPD |
| | (19) | | | KNKEPGAEEKFKEIAEAYDVLSDPRKREIFDRYG |
| | | | | EEGLKGSGGGGSSNWKWWPGIFDLEGGGGSSNWK |
| | | | | WWPGIFDLE |
| 53 | Flag-QBP1-JB1- | 141 | 149 | MDYKDDDDKGGTGSSNWKWWPGIFDEFMGKDYYQ |
| | QBP1-Flag | | | TLGLARGASDEEIKRAYRRQALRYHPDKNKEPGA |
| | | | | EEKFKEIAEAYDVLSDPRKREIFDRYGEEGLKGS |
| | | | | GGGGSGGGGSGGGGSGGGGSAAASNWKWWPGIFD |
| | | | | LESRGDYKDDDDK |
| 54 | QBP1-JB1-QBP1 | 142 | 106 | MSNWKWWPGIFDGKDYYQTLGLARGASDEEIKRA |
| | (1) | | | YRRQALRYHPDKNKEPGAEEKFKEIAEAYDVLSD |
| | | | | PRKREIFDRYGEEGLKGSSNWKWWPGIFDGDYKD |
| | | | | DDDK |
| 55 | QBP1-JB1-QBP1 | 143 | 105 | MSNWKWWPGIFDLEGKDYYQTLGLARGASDEEIK |
| | (2) | | | RAYRRQALRYHPDKNKEPGAEEKFKEIAEAYDVL |
| | | | | SDPRKREIFDRYGEEGLKGSGGGSSNWKWWPGIF |
| | | | | DLE |
| 56 | JB1-JB1-QBP1 | 144 | 201 | MGKDYYQTLGLARGASDEEIKRAYRRQALRYHPD |
| | | | | KNKEPGAEEKFKEIAEAYDVLSDPRKREIFDRYG |
| | | | | EEGLKGSGSEFMGKDYYQTLGLARGASDEEIKRA |
| | | | | YRRQALRYHPDKNKEPGAEEKFKEIAEAYDVLSD |
| | | | | PRKREIFDRYGEEGLKGSGGGGSGGGGSGGGGSG |
| | | | | GGGSAAASNWKWWPGIFDLESRGDYKDDDDK |
| 57 | JB1-QBP1-JB1- | 145 | 212 | MGKDYYQTLGLARGASDEEIKRAYRRQALRYHPD |
| | QBP1 | | | KNKEPGAEEKFKEIAEAYDVLSDPRKREIFDRYG |
| | | | | EEGLKGSGSSNWKWWPGIFDEFMGKDYYQTLGLA |
| | | | | RGASDEEIKRAYRRQALRYHPDKNKEPGAEEKFK |
| | | | | EIAEAYDVLSDPRKREIFDRYGEEGLKGSGGGGS |
| | | | | GGGGSGGGGSGGGGSAAASNWKWWPGIFDLESRG |
| | | | | DYKDDDDK |
| 58 | JB6-QBP1-QBP1 | 146 | 137 | MVDYYEVLGVQRHASPEDIKKAYRKLALKWHPDK |
| | | | | NPENKEEAERKFKQVAEAYEVLSDAKKRDIYDKY |
| | | | | GKEGLNGGDIGGGGSGGGGSGGGGSAAASNWKWW |
| | | | | PGIFDLEGDYKDDDDKGSRAAASNWKWWPGIFDL |
| | | | | E |
| 59 | JB13-QBP1-QBP1 | 147 | 136 | MGQDYYSVLGITRNSEDAQIKQAYRRLALKHHPL |
| | | | | KSNEPSSAEIFRQIAEAYDVLSDPMKRGIYDKFG |
| | | | | EEGLKGGDIGGGGSGGGGSGGGGSAAASNWKWWP |
| | | | | GIFDLEGDYKDDDDKGSRAAASNWKWWPGIFDLE |
| 60 | JA1-QBP1-QBP1 | 148 | 136 | MVKETTYYDVLGVKPNATQEELKKAYRKLALKYH |
| | | | | PDKNPNEGEKFKQISQAYEVLSDAKKRELYDKGG |
| | | | | EQAIKEGDIGGGGSGGGGSGGGGSAAASNWKWWP |
| | | | | GIFDLEGDYKDDDDKGSRAAASNWKWWPGIFDLE |
| 61 | JC7-QBP1-QBP1 | 149 | 133 | MDYYKILGVDKNASEDEIKKAYRKRALMHHPDRH |
| | | | | SGASAEVQKEEEKKFKEVGEAFTILSDPKKKTRY |
| | | | | DSGQDIGGGGSGGGGSGGGGSAAASNWKWWPGIF |
| | | | | DLEGDYKDDDDKGSRAAASNWKWWPGIFDLE |
| 62 | DnaJ-QBP1- | 150 | 133 | MAKQDYYEILGVSKTAEEREIRKAYKRLAMKYHP |
| | QBP1 | | | DRNQGDKEAEAKFKEIKEAYEVLTDSQKRAAYDQ |
| | | | | YGHADIGGGGSGGGGSGGGGSAAASNWKWWPGIF |
| | | | | DLEGDYKDDDDKGSRAAASNWKWWPGIFDLE |
| 63 | SV40-QBP1- | 151 | 126 | MQLMDLLGLERSAWGNIPLMRKAYLKKCKEFHPD |
| | QBP1 | | | KGGDEEKMKKMNTLYKKMEDGVKYAHQPDFGDIG |
| | | | | GGGSGGGGSGGGGSAAASNWKWWPGIFDLEGDYK |
| | | | | DDDDKGSRAAASNWKWWPGIFDLE |
| 64 | JB1QBP1 (1) | 152 | 111 | MGKDYYQTLGLARGASDEEIKRAYRRQALRYHPD |
| | | | | KNKEPGAEEKFKEIAEAYDVLSDPRKREIFDRYG |
| | | | | EEGLKGSDIGGGGSGGGGSGGGGSGGGGSAAASN |
| | | | | WKWWPGIFD |
| 65 | JB1QBP1 (2) | 153 | 88 | MGKDYYQTLGLARGASDEEIKRAYRRQALRYHPD |
| | | | | KNKEPGAEEKFKEIAEAYDVLSDPRKREIFDRYG |
| | | | | EEGLKGSSNWKWWPGIFDLE |
| 66 | JB1QBP1 (3) | 154 | 92 | MGKDYYQTLGLARGASDEEIKRAYRRQALRYHPD |
| | | | | KNKEPGAEEKFKEIAEAYDVLSDPRKREIFDRYG |
| | | | | EEGLKGSGGGSSNWKWWPGIFDLE |
| 67 | JB1QBP1 (4) | 155 | 93 | MGKDYYQTLGLARGASDEEIKRAYRRQALRYHPD |
| | | | | KNKEPGAEEKFKEIAEAYDVLSDPRKREIFDRYG |
| | | | | EEGLKGSEAAAKSNWKWWPGIFDLE |
| 68 | JB1QBP1 (5) | 156 | 86 | MGKDYYQTLGLARGASDEEIKRAYRRQALRYHPD |
| | | | | KNKEPGAEEKFKEIAEAYDVLSDPRKREIFDRYG |
| | | | | EEGLKGSSNWKWWPGIFD |
| 69 | JB1QBP1 (6) | 157 | 90 | MGKDYYQTLGLARGASDEEIKRAYRRQALRYHPD |
| | | | | KNKEPGAEEKFKEIAEAYDVLSDPRKREIFDRYG |
| | | | | EEGLKGSGGGSSNWKWWPGIFD |
| 70 | JB1QBP1 (7) | 158 | 91 | MGKDYYQTLGLARGASDEEIKRAYRRQALRYHPD |
| | | | | KNKEPGAEEKFKEIAEAYDVLSDPRKREIFDRYG |
| | | | | EEGLKGSEAAAKSNWKWWPGIFD |

* Constructs containing 2X or 3X indicate the presence of 2 or 3 tandem copies of the polyglutamine-binding domains. For example, JB1-2XQBP1 indicates two tandem copies of the QBP1 (SEQ ID NO:57).

II. Nucleic Acids Encoding Fusion Protein Constructs

According to another aspect of the invention, provided are isolated nucleic acids comprising a polynucleotide sequence selected from (a) a polynucleotide encoding the fusion protein of any of the foregoing embodiments, or (b) the complement of the polynucleotide of (a). The present invention provides isolated nucleic acids encoding fusion proteins comprising the J domain and polyglutamine-binding domain, and sequences complementary to such nucleic acid molecules encoding the fusion proteins, including homologous variants thereof. In another aspect, the invention encompasses methods to produce nucleic acids encoding the fusion proteins disclosed herein, and sequences complementary to the nucleic acid molecules encoding fusion proteins, including homologous variants thereof. The nucleic acid according to this aspect of the invention can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In yet another aspect, disclosed is a method of producing a fusion protein comprising (a) synthesizing and/or assembling nucleotides encoding the fusion protein, (b) incorporating the encoding gene into an expression vector appropriate for a host cell, (c) transforming the appropriate host cell with the expression vector, and (d) culturing the host cell under conditions causing or permitting the fusion protein to be expressed in the transformed host cell, thereby producing the biologically-active fusion protein, which is recovered as an isolated fusion protein by standard protein purification methods known in the art. Standard recombinant techniques in molecular biology is used to make the polynucleotides and expression vectors of the present invention.

In accordance with the invention, nucleic acid sequences that encode the fusion proteins disclosed herein (or its complement) are used to generate recombinant DNA molecules that direct the expression of the fusion proteins in appropriate host cells. Several cloning strategies are suitable for performing the present invention, many of which is used to generate a construct that comprises a gene coding for a fusion protein of the present invention, or its complement. In some embodiments, the cloning strategy is used to create a gene that encodes a fusion protein of the invention, or their complement.

In certain embodiments, a nucleic acid encoding one or more fusion proteins is an RNA molecule, and can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In various embodiments, the nucleic acid is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a comprise a 5′ cap or modified 5′ cap and/or a poly(A) sequence. As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from RNases. The capping moiety can be modified to modulate functionality of mRNA such as its stability or efficiency of translation. In a particular embodiment, the mRNA comprises a poly(A) sequence of between about 50 and about 5000 adenines. In one embodiment, the mRNA comprises a poly (A) sequence of between about 100 and about 1000 bases, between about 200 and about 500 bases, or between about 300 and about 400 bases. In one embodiment, the mRNA comprises a poly (A) sequence of about 65 bases, about 100 bases, about 200 bases, about 300 bases, about 400 bases, about 500 bases, about 600 bases, about 700 bases, about 800 bases, about 900 bases, or about 1000 or more bases. poly(A) sequences can be modified chemically or enzymatically to modulate mRNA functionality such as localization, stability or efficiency of translation.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

In certain embodiments, the nucleic acid sequence comprises a nucleotide sequence encoding the gene of interest (e.g., the fusion proteins comprising a J domain and a polyglutamine binding domain) within a nucleic acid cassette. The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Illustrative ubiquitous expression control sequences suitable for use in particular embodiments include, but are not limited to, a cytomegalovirus (CMV) immediate early promoter, a viral simian virus 40 (SV40) (e.g early or late), a Moloney murine leukemia virus (MoMLV) LTR promoter, a Rous sarcoma virus (RSV) LTR, a herpes simplex virus (HSV) (thymidine kinase) promoter, H5, P7.5, and PI I promoters from vaccinia virus, an elongation factor 1-alpha (EFIa) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), ß-kinesin (b-KIN), the human ROSA 26 locus Orions et al, Nature Biotechnology 25, 1477-1482 (2007)), a Ubiquitin C promoter (UBC), a phosphogly cerate kinase-1 (PGK) promoter, a cytomegalovirus enhancer/chicken ß-actin (CAG) promoter (Okabe et al. (1997) FEBS let. 407: 313-9), a b-actin promoter and a myeloproliferative sarcoma virus enhancer, negative control region deleted, dl587rev primer binding site substituted (MND) U3 promoter (Haas et al., Journal of Virology. 2003; 77(17): 9439-9450).

In one embodiment, at least one element may be used with the polynucleotides described herein to enhance the transgene target specificity and expression (See e.g., Powell et al. (2015) Discovery Medicine 19(102):49-57, the contents of which are herein incorporated by reference in its entirety) such as promoters. Promoters for which promote expression in most tissues include, but are not limited to, human elongation factor la-subunit (EFIa), immediate-early cytomegalovirus (CMV), chicken β-actin (CBA) and its derivative CAG, the β glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), the synapsin (Syn), the methyl-CpG binding protein 2 (MeCP2), CaMKII, mGluR2, NFL, NFH, ηβ2, PPE, Enk and EAAT2 promoters. A non-limiting example of a tissue-specific expression elements for astrocytes include the glial fibrillary acidic protein (GFAP) and EAAT2 promoters. A non-limiting example of a tissue-specific expression element for oligodendrocytes include the myelin basic protein (MBP) promoter. Yu et al. (2011) Molecular Pain, 7:63, incorporated by reference in its entirety) evaluated the expression of eGFP under the CAG, EFIa, PGK and UBC promoters in rat DRG cells and primary DRG cells using lentiviral vectors and found that UBC showed weaker expression than the other 3 promoters and there was only 10-12% glia expression seen for all promoters. Soderblom et al. (E. Neuro 2015, incorporated by reference in its entirety) the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al, (2001) Gene Therapy, Vol. 8, 1539-1546; incorporated by reference in its entirety). Husain et al. (2009) Gene Therapy, incorporated by reference in its entirety) evaluated a HβH construct with a hGUSB promoter, a HSV-1LAT promoter and a NSE promoter and found that the HβH construct showed weaker expression than NSE in mice brain. Passini and Wolfe (J. Virol. 2001, 12382-12392, incorporated by reference in its entirety) evaluated the long term effects of the H1H vector following an intraventricular injection in neonatal mice and found that there was sustained expression for at least 1 year. Low expression in all brain regions was found by Xu et al. (2001) Gene Therapy, 8, 1323-1332; incorporated by reference in its entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. 2007 and Raymond et al. 2004; incorporated by reference in its entirety).

III. Vectors Comprising Nucleic Acids Encoding Fusion Proteins

Also provided is a vector comprising nucleic acid according to the invention. Such a vector preferably comprises additional nucleic acid sequences such as elements necessary for transcription/translation of the nucleic acid sequence encoding a phosphatase (for example promoter and/or terminator sequences). Said vectors can also comprise nucleic acid sequences coding for selection markers (for example an antibiotic) to select or maintain host cells transformed with said vector. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to an affected cell (e.g. neuronal cells) In one embodiment, the vector is an in vitro synthesized or synthetically prepared mRNA encoding a fusion protein comprising a J domain and a polyglutamine-binding domain. Illustrative examples of non-viral vectors include, but are not limited to mRNA, plasmids (e.g., DNA plasmids or RNA plasmids), transposons, cosmids, and bacterial artificial chromosomes.

Illustrative examples of vectors include, but are not limited to, a plasmid, autonomously replicating sequences, and transposable elements, e.g., piggyBac, Sleeping Beauty, Mosl, Tcl/mariner, Toll, mini-Tol2, Tc3, MuA, Himar I, Frog Prince, and derivatives thereof. Additional Illustrative examples of vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Illustrative examples of viruses useful as vectors include, without limitation, retrovirus (including lentivirus), adenovirus, adeno-associated virus, herpesvirus (e.g., herpes simplex vims), poxvirus, baculovirus, papillomavirus, and papovavirus (e.g., SV40). Illustrative examples of expression vectors include, but are not limited to, pClneo vectors (Promega) for expression in mammalian cells; pLenti4/V 5-DEST™, pLenti6/V 5-DEST™, and pLenti6.2/V 5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Suitable recognition sites for the FLP recombinase include, but are not limited to: FRT (McLeod, et al., 1996), FI, F2, F3 (Schlake and Bode, 1994), FyFs (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme I Integrase, e.g., phi-c3I. The (pC3I SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by ϕ031 homodimers (Groth et al., 2000). The product sites, attL and attR, are effectively inert to further tpQA 1-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a fusion protein comprising a J domain and polyglutamine-binding domain. Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides encoding fusion protein comprising a J domain and a polyglutamine-binding domain are introduced into a cell, e.g., a neuronal cell, by transducing the cell with a recombinant adeno-associated virus (rAAV), comprising the one or more polynucleotides. AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2 (described, for example, in U.S. Pat. No. 6,962,815B2, which is incorporated herein by reference in its entirety), AAV3, AAV4, AAV5 (described, for example, in U.S. Pat. No. 7,479,554B2, which is incorporated herein by reference in its entirety), AAV6, AAV7, AAV8 (described, for example, in U.S. Pat. No. 7,282,199B2, which is incorporated herein by reference in its entirety), AAV9 (described, for example, in U.S. Pat. No. 9,737,618B2, which is incorporated herein by reference in its entirety), AAV rh10 (described, for example, in U.S. Pat. No. 9,790,472B2, which is incorporated herein by reference in its entirety) or AAV 10. In one embodiment, the vector of the present invention is encapsulated into a capsid selected from the group consisting of AAV2, AAV5, AAV8, AAV9 and AAV rh10. In one embodiment, the vector is encapsulated in AAV2. In one embodiment, the vector is encapsulated in AAV5. In one embodiment, the vector is encapsulated in AAV8. In one embodiment, the vector is encapsulated in AAV9. In still one embodiment, the vector is encapsulated in AAV rh10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

IV. Delivery

In particular embodiments, one or more polynucleotides encoding a fusion protein comprising a J domain and polyglutamine-binding domain are introduced into a cell by non-viral or viral vectors. Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, poly cation or lipidnucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al., (2003) Gene Therapy. 10: 180-187; and Balazs et al., (20W) Journal of Drug Delivery. 2011:1-12. Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion), by intrathecal injection, intracerebroventricular injection or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding a fusion protein disclosed herein is administered directly to an organism for transduction of cells in vivo.

A viral vector, suitably packaged and formulated, can be delivered into the central nervous system (CNS) via intrathecal delivery. For example, adeno-associated viral vectors can be delivered using methods described in U.S. Ser. No. 15/771,481, which is incorporated herein by reference in its entirety.

Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

In various embodiments, one or more polynucleotides encoding a fusion protein disclosed herein are introduced into a cell, for example, a neuronal cell or neuronal stem cell, by transducing the cell with a retrovirus, e.g., lentivirus, comprising the one or more polynucleotides. As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex vims (HSV) (thymidine kinase) promoters. In certain embodiments, lentiviral vectors are produced according to known methods. See e.g., Kutner et al., BMC Biotechnol. 2009; 9:10. doi: 10.1186/1472-6750-9-10; Kutner et al., Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a fusion protein disclosed herein are introduced into a target cell by transducing the cell with an adenovirus comprising the one or more polynucleotides. Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad EIa, EIb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7: 1083-9 (1998)).

In various embodiments, one or more polynucleotides encoding a fusion protein of the invention are introduced into the target cell of a subject by transducing the cell with a herpes simplex virus, e.g., HSV-I, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which is incorporated by reference herein in its entirety.

V. Cells Expressing the Fusion Protein

In yet another aspect, the invention provides for cells expressing the fusion proteins described herein. Cells can be transfected with a vector encoding the fusion protein as described herein above. In one embodiment, the cell is a prokaryotic cell. In another embodiment, the cell is a eukaryotic cell. In still another embodiment, the cell is a mammalian cell. In a particular embodiment, the cell is a human cell. In another embodiment, the cell is a human cell that is derived from a patient that suffers from, or is at risk of suffering from, a polyglutamine repeat disorder. The cell can be a neuronal cell or a muscle cell.

Cells expressing the fusion protein can be useful in producing the fusion protein. In this embodiment, the cells are transfected with a vector overexpressing the fusion protein. The fusion protein may optionally contain an epitope, for example, a human Fc domain or a FLAG epitope, as described herein above, that would facilitate the purification (using a Protein A- or anti-FLAG antibody column, respectively). The epitope may be connected to the rest of the fusion protein via a linker or a protease substrate sequence such that, during or after purification, the epitope can be removed from the fusion protein.

Cells expressing the fusion protein can also be useful in a therapeutic context. In one embodiment, cells are collected from a patient in need of therapy (e.g., a patient who suffers from or is at risk of suffering from a polyglutamine repeat disorder). In one embodiment, the cells are neuronal cells. Collected cells are then transfected with a vector expressing the fusion protein. The transfected cells can then be processed to enrich or select for transfected cells. The transfected cells can also be treated to differentiate into a different type of cell, for example, a neuronal cell. After processing, the transfected cells can be administered to the patient. In one embodiment, the cells are administered by directed injection into the central nervous system by intrathecal injection, intracranial injection or intracerebroventricular injection.

In an alternative embodiment, cells expressing a secreted form of the fusion protein can be used. For example, fusion protein constructs can be designed having a signal sequence on the N-terminal end. Representative signal sequences are shown below in Table 6.

TABLE 6
Representative Signal Sequences

| SEQ ID NO: | SEQUENCE |
|---|---|
| 159 | MGVKVLFALICIAVAEA |
| 160 | MAPVQLLGLLVLFLPAMRC |
| 161 | MAVLGLLFCLVTFPSCVLS |

Therefore, in one embodiment, the fusion protein comprises a signal sequence and a fusion protein, wherein the signal sequence is selected from the group consisting of SEQ ID NOs: 159-161, and the fusion protein is selected from the group consisting of SEQ ID NOs: 89-158. In another embodiment, the fusion protein comprises the signal sequence of SEQ ID NO: 116, and the fusion protein selected from the group consisting of SEQ ID NOs: 89-158. Cells expressing the fusion protein constructs with the signal sequence can be administered to a subject, for example a human subject (e.g., a patient having or at risk of suffering from a polyglutamine repeat disorder). The fusion protein is secreted from the cells, which help reduce polyglutamine repeat-containing protein aggregation and/or associated cytotoxicity.

As described herein above, in certain embodiments, the fusion protein can further comprise a cell-penetrating peptide. A cell expressing a fusion protein comprising a signal sequence and a cell-penetrating peptide would be capable of secreting the fusion protein, devoid of the signal sequence. The secreted fusion protein, also comprising the cell-penetrating peptide, would then be capable of entering nearby cells, and have the potential to reduce aggregation and/or cytotoxicity mediated by polyglutamine repeat proteins in those cells.

VI. Methods of Use

In another aspect, the invention provides a method for achieving a beneficial effect in disorders and/or in a polyglutamine-repeat disease, disorder or condition mediated by polyglutamine repeat associated protein aggregation. The polyglutamine repeat disease is selected from the group consisting of Huntington's disease, SCA type 1, SCA type 2, SCA type 6, SCA type 7, SCA type 17, MJD/SCA3, DRPLA, and SBMA. In one embodiment, the polyglutamine repeat disease is Huntington's disease.

In some embodiments, the invention provides methods for treating a subject, such as a human, with a polyglutamine repeat disease, disorder or condition comprising the step of administering to the subject a therapeutically- or prophylactically-effective amount of a fusion protein, a nucleic acid encoding such fusion protein, or a viral vector encoding such fusion protein described herein, wherein said administration results in the improvement of one or more biochemical or physiological parameters or clinical endpoints associated with the polyglutamine repeat disease, disorder or condition.

In other embodiments, the invention provides for a method of reducing aggregation of polyglutamine-containing proteins in a cell. The cell can be a cultured cell or an isolated cell. The cell can also be from a subject, for example, a human subject. In one embodiment, the cell is in the central nervous system of the human subject. In another embodiment, the human subject is suffering from, or is at risk of suffering from a polyglutamine repeat disease, including, but not limited to, Huntington's disease, SCA type 1, SCA type 2, SCA type 6, SCA type 7, SCA type 17, MJD/SCA3, DRPLA, and SBMA.

Aggregation of polyglutamine-containing proteins can be detected in a number of ways. In one example, aggregated polyglutamine-containing proteins can be distinguished from free (i.e., soluble) polyglutamine-containing proteins based on solubility, for example, by trapping the insoluble aggregates by passage of cellular lysates through a selective filter. Non-aggregated proteins pass through these filters, whereas aggregates will be retained on the filter, which can be detected using any number of reagents, including antibodies directed against the polyglutamine-containing protein. The amount of trapped aggregated protein in the lysate of a cell sample treated with the fusion protein or a nucleic acid, vector, or viral particle encoding the fusion protein as described herein can be compared with lysates from an untreated or control-treated cell, where a reduction in the amount of aggregated polyglutamine-containing protein in the treated sample when compared with the control sample is indicative of efficacy of the fusion protein or a nucleic acid, vector, or viral particle encoding the fusion protein (see, for example, Kim et al., (2014) Mol. Cell. Biol., 34: 643-652, and Example 1). A greater reduction in the aggregated polyglutamine-containing protein when compared with controls indicates a higher potency. Reduction of aggregation of polyglutamine-containing proteins can also be detected directly in the cell, for example, using immunofluorescence microscopy with labeled reagents detecting the polyglutamine-containing protein (see, for example, Difiglia et al., (1997) Science, 277: 1990-1993, and Example 1). For example, mutant (polyglutamine expanded) huntingtin is found localized to neuronal inclusions and dystrophic neurites in the HD cortex and striatum of patients with Huntington's disease.

Therefore, in one embodiment, the method comprises contacting the cell with an amount of the fusion protein or a nucleic acid, vector, or viral particle encoding the fusion protein effective to reduce aggregation of polyglutamine-containing proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

In yet another aspect, the compositions described herein can be used in a method of reducing protein aggregation associated with a disease selected from the group consisting of ALS, FTD, Parkinson's disease, Huntington's disease, Alzheimer's disease, hippocampal sclerosis, and dementia with Lewy's bodies. As described in the Examples section, a number of constructs described herein have been tested in vitro and found to reduce pathological aggregation of mutant forms of TDP-43 and SOD1, which are associated with these diseases. As such, in one embodiment, the method comprises contacting the cell with an amount of the fusion protein, nucleic acid, vector, or viral particle encoding the fusion protein described herein, in an amount effective to reduce aggregation of TDP-43 by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell. In another embodiment, a method of reducing aggregation of SOD1 is provided: the method comprises contacting the cell with an amount of the fusion protein, nucleic acid, vector, or viral particle encoding the fusion protein described herein, in an amount effective to reduce aggregation of SOD1 by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

VII. Pharmaceutical Compositions

The compositions contemplated herein may comprise one or more fusion protein comprising a J domain and polyglutamine-binding domain, polynucleotides encoding such fusion proteins, vectors comprising same, genetically modified cells, etc., as contemplated herein. Compositions include, but are not limited to pharmaceutical compositions. A “pharmaceutical composition” refers to a composition formulated in pharmaceutically acceptable or physiologically acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier”, “diluent” or “excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

VIII. Dosages

The dosage of the compositions (e.g., a composition including a fusion protein construct, nucleic acid or gene therapy viral particle) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. The compositions described herein can be administered initially in a suitable dosage that can be adjusted as required, depending on the clinical response. In some aspects, the dosage of a composition is a prophylactically or a therapeutically effective amount.

IX. Kits

Kits including (a) a pharmaceutical composition including a fusion protein construct, nucleic acid encoding such fusion protein, or viral particle encompassing such nucleic acid that reduces aggregation of polyglutamine repeat proteins in a cell or subject described herein, and (b) a package insert with instructions to perform any of the methods described herein are contemplated. In some aspects, the kit includes (a) a pharmaceutical composition including a composition described herein that reduces the aggregation of polyglutamine repeat proteins in a cell or subject described herein, (b) an additional therapeutic agent, and (c) a package insert with instructions to perform any of the methods described herein.

### EXAMPLES

To test whether J domains can be specifically engineered to facilitate the proper folding of aggregated proteins, we designed and tested a number of fusion protein constructs designed to target polyglutamine repeats in HTT proteins.

### Example 1: Fusion Protein Design

A. Methods

### General Techniques and Materials

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3rd edition, Cold Spring Harbor Laboratory Press, 2001; “Current protocols in molecular biology”, F. M. Ausubel, et al., eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11th Edition, McGraw-Hill, 2005; and Freshney, R. I., “Culture of Animal Cells: A Manual of Basic Technique,” 4th edition, John Wiley & Sons, Somerset, N J, 2000, the contents of which are incorporated in their entirety herein by reference. HEK-293 cells (human embryonic kidney cells) were purchased from the American Type Culture Collection (Manassas, Va.). anti-FLAG antibody was purchased from Thermo Fisher Scientific. Rabbit anti-GFP antibody was purchased from GenScripts (Piscataway, N.J.). For ease of purification, detection and characterization, some of the fusion protein constructs used in these Examples may contain, in addition to the sequences provided in SEQ ID NOs: 89-158, linker sequences and/or an epitope, such as the FLAG epitope of SEQ ID NO:163 at the C-terminus of the protein.

### Expression and Detection of Proteins in HEK293 Cells

Expression vector plasmids encoding various protein constructs were transfected into HEK293 cells with Lipofectamine 3000 transfection reagent (Thermo Fisher Scientific). Cell lysates were analyzed for expressed proteins using immunoblot assays. Samples of culture media were centrifuged to remove debris prior to analysis. Cells were lysed in a lysis buffer (10 mM Tris-HCl, pH 8.0, 150 mM NaCl, 10 mM EDTA, 2% SDS) containing 2 mM PMSF and protease cocktail (Complete Protease Inhibitor Cocktail; Sigma). After brief sonication, the samples were analyzed for expressed proteins using immunoblot assays. For immunoblot analysis, samples were boiled in an SDS-sample buffer and run on polyacrylamide electrophoresis. Thereafter, the separated protein bands were transferred to a PVDF membrane.

Expressed proteins were detected using a chemiluminescent signal. Briefly, blots were reacted with a primary antibody capable of binding the particular epitope (e.g., GFP). After rinsing away the unreacted primary antibody, a secondary, enzyme-linked antibody (e.g., HRP-linked anti-IgG antibody) was allowed to react with the primary antibody molecules bound to the blots. Following rinsing, a chemiluminescent reagent was added, and the resultant chemiluminescent signals in the blots were captured on X-ray film.

### Filter Trap Assay

HEK293 cells that were transfected with GFP-HTT(Q23) or GFP-HTT(Q74) were homogenized in SDS-lysis buffer (10 mM Tris, pH8.0, 150 mM NaCl, 2% SDS). After brief sonication, protein concentration was measured by BCA assay kit (Pierce). The equal protein amount was applied to 0.22 um cellulose acetate membrane under negative pressure in a filter trap apparatus. After the wash of the membrane, the trapped proteins (aggregated proteins) were detected by immunoblot assay using anti-GFP antibody.

### Fluorescence Microscopy

In some instances, aggregation of polyglutamine-containing GFP reporter constructs (described below) were detected in vivo using fluorescence microscopy. Cultured cells expressing the reporter constructs as well as the fusion protein comprising the i domain and polyglutamine-binding domain were washed with PBS and fixed with 4% paraformaldehyde in PBS for 5 minutes. After three 5-min washes with PBS, nuclear DNA was stained with DAPI. Percentage of cells containing mHtt aggregates (GFP foci) in transfected cells was counted.

B. Reporter Constructs

In order to test the ability of various fusion protein constructs to reduce aggregation of polyglutamine-containing proteins, HEK293 cells expressing GFP-based reporter constructs were generated.

HEK293 cells were cultured and transfected with the plasmids encoding reporter constructs GFP-HTT Q23 or GFP-HTT Q74, containing the amino acids within the HTT exon 1 with either 23 or 74 polyQ tracts, respectively, fused to the C-terminus of GFP via a 13 amino acid linker, as shown below in Table 7.

pcDNA3 (Life Technologies, Grand Island, N.Y.) with a modified multiple cloning site (MCS) was used as a backbone plasmid. DNA molecules encoding protein sequences were obtained by polymerase chain reaction (PCR), gene synthesis, or the annealing of complementary DNA molecules with standard protocols. DNA molecules encoding the amino acid sequence of HTT(Q23) and HTT(Q74) were synthesized (BioBasic, Canada). DNA molecules having a DNA sequence, GGAGGCGGAGGCAGTGGTGGTGGGGGAAGCGGTGGAGGTGGAAGC and GCCGAGGCGGCGGCCAAGGAGGCCGCCGCCAAG, encoding the glycine-serine linker sequence (GGGGSGGGGSGGGGS), and rigid linker sequence (AEAAAKEAAAK), respectively, were obtained by annealing complementary single strands synthesized by standard methods. The cDNA sequence encoding the respective linker was inserted into the backbone plasmid.

TABLE 7
HTT Reporter Constructs

| Construct | SEQ | | |
|---|---|---|---|
| Name | ID NO: | Length | Sequence |
| GFP- | 169 | 341 | MVSKGEELFTGVVPILVELD |
| HTTQ23 | | | GDVNGHKFSVSGEGEGDATY |
| | | | GKLTLKFICTTGKLPVPWPT |
| | | | LVTTLTYGVQCFSRYPDHMK |
| | | | QHDFFKSAMPEGYVQERTIF |
| | | | FKDDGNYKTRAEVKFEGDTL |
| | | | VNRIELKGIDFKEDGNILGH |
| | | | KLEYNYNSHNVYIMADKQKN |
| | | | GIKVNFKIRHNIEDGSVQLA |
| | | | DHYQQNTPIGDGPVLLPDNH |
| | | | YLSTQSALSKDPNEKRDHMV |
| | | | LLEFVTAAGITLGMDELYKS |
| | | | GRTQISSSSFEFATLEKLMK |
| | | | AFESLKSFQQQQQQQQQQQQ |
| | | | QQQQQQQQQQQPPPPPPPPP |
| | | | PPQLPQPPPQAQPLLPQPQP |
| | | | PPPPPPPPPGPAVAEEPLHR |
| | | | P |
| GFP- | 170 | 392 | MVSKGEELFTGVVPILVELD |
| HTTQ74 | | | GDVNGHKFSVSGEGEGDATY |
| | | | GKLTLKFICTTGKLPVPWPT |
| | | | LVTTLTYGVQCFSRYPDHMK |
| | | | QHDFFKSAMPEGYVQERTIF |
| | | | FKDDGNYKTRAEVKFEGDTL |
| | | | VNRIELKGIDFKEDGNILGH |
| | | | KLEYNYNSHNVYIMADKQKN |
| | | | GIKVNFKIRHNIEDGSVQLA |
| | | | DHYQQNTPIGDGPVLLPDNH |
| | | | YLSTQSALSKDPNEKRDHMV |
| | | | LLEFVTAAGITLGMDELYKS |
| | | | GRTQISSSSFEFATLEKLMK |
| | | | AFESLKSFQQQQQQQQQQQQ |
| | | | QQQQQQQQQQQQQQQQQQQQ |
| | | | QQQQQQQQQQQQQQQQQQQQ |
| | | | QQQQQQQQQQQQQQQQQQQQ |
| | | | QQPPPPPPPPPPPQLPQPPP |
| | | | QAQPLLPQPQPPPPPPPPPP |
| | | | GPAVAEEPLHRP |

C. Fusion Protein Constructs

i. Domain Arrangement

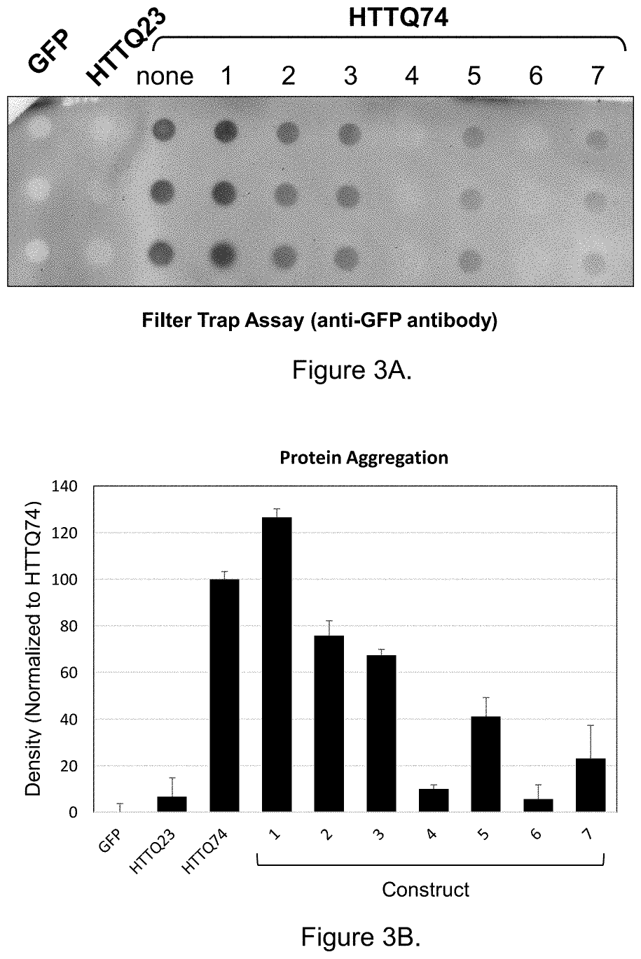

In order to determine the optimal configuration of the fusion protein, several fusion protein constructs were generated comprising the J domain sequence derived from a human Hsp40 (DnaJB1, or SEQ ID NO:5), and the polyglutamine binding peptide 1 (QBP1), a 11 aa synthetic peptide (SEQ ID NO:57) that was identified by a combinatorial screening approach for its specific binding affinity to the expanded polyQ tract but not to the polyQ motif found in normal HTT 32. A number of initial fusion protein constructs were designed (SEQ ID NOs: 89-93), altering the position of QBP1 relative to the J domain (i.e., attached to the N-terminus or C-terminus of the J domain, with or without an optional linker). Two constructs, named JB1-QBP1 (Construct 2, SEQ ID NO: 90) and QBP1-JB1 (Construct 3, SEQ ID NO: 91), both contain a single QBP1 (SEQ ID NO:57) attached to the C-terminus and N-terminus of the J domain from human DnaJB1 (SEQ ID NO:5), respectively, via a short linker sequence. Vectors encoding these fusion protein constructs were transfected into cells, along with the reporter constructs described above. The extent of protein aggregation of these constructs, as determined using the filter trap assay, was compared with those in cells expressing the short polyglutamine repeat (GFP-HTTQ23) reporter construct alone, the long repeat (GFP-HTTQ74) reporter alone, as well as cells expressing the long repeat reporter and a construct comprising the J domain from human DnaJB1 but no polyglutamine-binding domain (Construct No. 1, SEQ ID NO: 89). As shown in FIG. 3 and also summarized below in Table 8, as expected, the long repeat (GFP-HTTQ74) reporter shows higher level of aggregation than the GFP-HTTQ23 reporter when measured using the filter trap assay. The detectable aggregation was not reduced by co-expression of the DnaJB1 control construct. However, co-expression with JB1-QBP1 (Construct 2) or QBP1-JB1 (Construct 3) resulted in 76% and 67% of the control aggregation levels, respectively.

TABLE 8
Relative Activity of Fusion Protein
Constructs in Reducing Aggregation

| | Fusion Protein | | |
|---|---|---|---|
| | Construct | | % Aggregation |
| | Number | | (relative to Q74 |
| Reporter | (SEQ ID NO:) | Construct Name | alone) |
| GFP | None | None | 0% |
| GFP-HTTQ23 | None | None | 6.65% |
| GFP-HTTQ74 | None | None | 100% |
| GFP-HTTQ74 | 1 (89) | JB1 only | 126.69% |
| GFP-HTTQ74 | 2 (90) | JB1-QBP1 | 75.87% |
| GFP-HTTQ74 | 3 (91) | QBP1-JB1 | 67.47% |
| GFP-HTTQ74 | 4 (92) | JB1-2XQBP1 | 9.86% |
| GFP-HTTQ74 | 5 (93) | JB1-3XQBP1 | 41.11% |
| GFP-HTTQ74 | 6 (94) | QBP1-JB1-QBP1 | 5.62% |
| GFP-HTTQ74 | 7 (95) | QBP1-JB1-2XQBP1 | 22.99% |

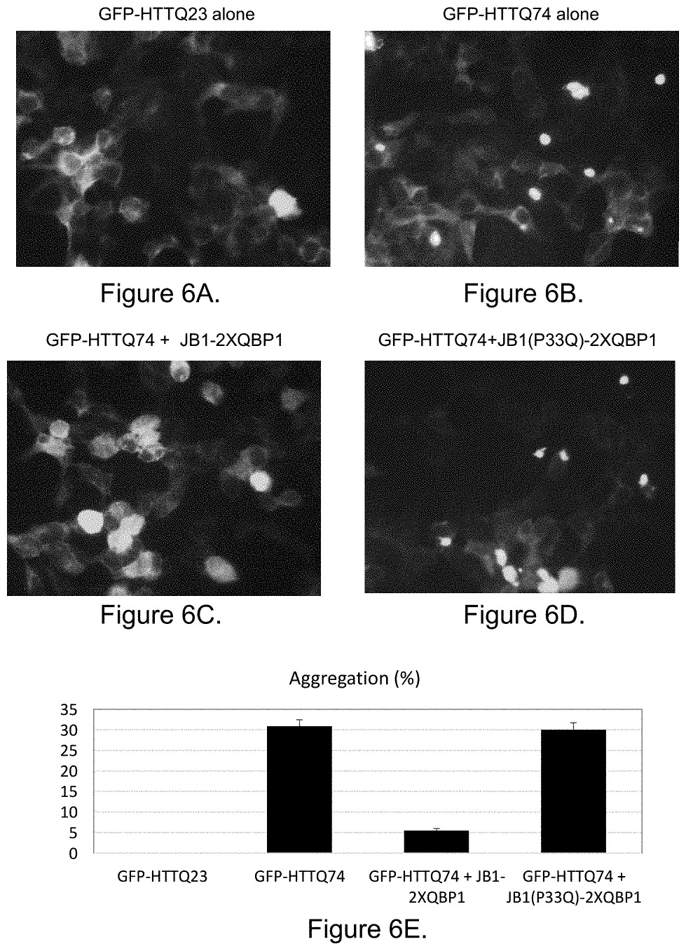

Cells expressing the reporter constructs were also observed by fluorescence microscopy. We found that GFP-HTTQ74 made conspicuous nuclear inclusions (FIG. 6a), while the GFP fluorescence of cells expressing the GFP-HTTQ23 reporter construct was distributed more diffusely throughout the cytoplasm (FIG. 6b), consistent with previously reported results. When we co-expressed GFP-HTTQ74 with the fusion protein construct 4 (JB1-2XQBP1), we observed that most of the aggregation disappeared (FIG. 6c). To eliminate the possibility of artifact, we inserted point mutation in “HPD” sequence of J domain as a negative control. As described below in section iii, the resulting construct 8 (JB1(P33Q)-2XQBP1, SEQ ID NO: 96) was found to have little to no ability to reduce aggregation by filter trap assay. Co-expression of construct 8 had no effect on GFP-HTTQ74 distribution, with conspicuous nuclear inclusions indistinguishable from GFP-HTTQ74 controls with no fusion protein construct (FIG. 6d, compared with 6a). Furthermore, the effect appears to be specific to expanded HTT since co-expression of construct 1, containing only the DnaJB1 J domain with no polyglutamine-binding domain, had no effect on reducing aggregation (not shown). Previous studies had indicated that fusion proteins consisting of CMA peptides fused to QBP1 was effective in selectively degrading mutant HTT protein in vitro (Bauer et al. (2010) Nat. Biotech. 28, 256-263). To test whether these constructs had comparable activity, we generated CMA peptides fused with either one or two tandem copies of the QBP1 peptide. However, CMA-based peptides failed to show appreciable reduction of HTT aggregation using the filter trap assay (data not shown).

ii. Multiple Polyglutamine-Binding Proteins

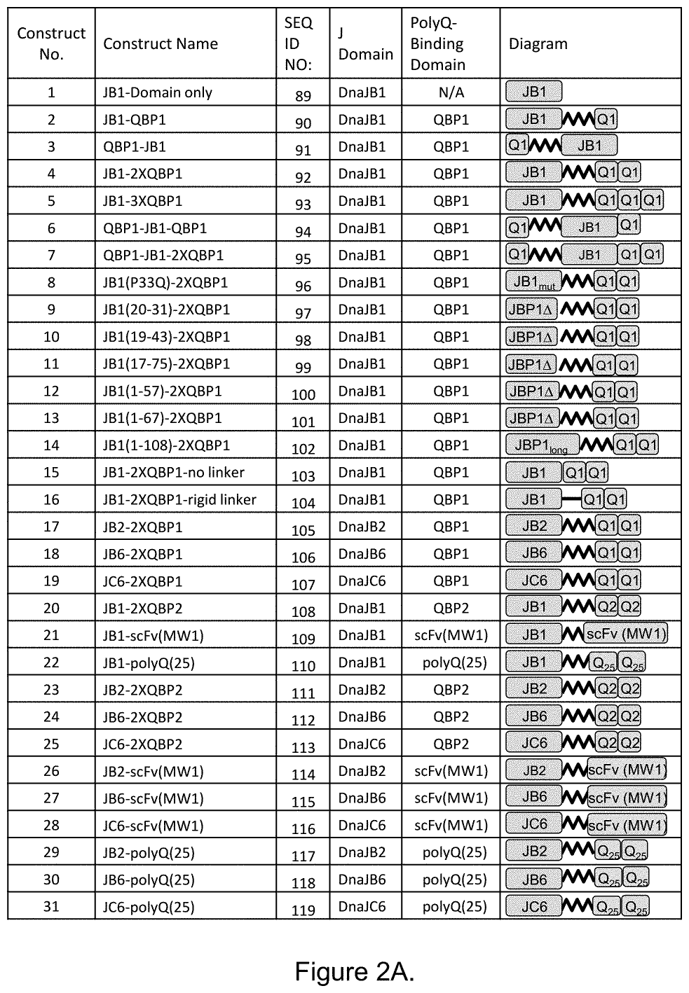

The following additional constructs were generated and tested for their ability to reduce aggregation of the HTTQ74 reporter construct (see FIG. 2):
Construct 4 (SEQ ID NO: 92), comprising two tandem repeats of the QBP1 attached to the C-terminal end of the DnaJB1 J domain;
Construct 5 (SEQ ID NO: 93), comprising three tandem repeats of the QBP1 attached to the C-terminal end of the DnaJB1 J domain;
Construct 6 (SEQ ID NO: 94), comprising the comprising the DnaJB1 J domain sandwiched on either side by QBP1; and,
Construct 7 (SEQ ID NO: 95), the DnaJB1 J domain and three copies of QBP1 in a QBP1-DnaJB1-QBP1-QBP1 arrangement.

As shown in FIG. 3, all fusion protein constructs containing at least two copies of a polyglutamine-binding domain resulted in greater reduction in aggregation. Constructs 4, 5, 6 and 7 were all capable of reducing aggregation by at least 50%, and Constructs 4 and 6 were capable of reducing aggregation by at least 90%.

iii. Requirements within J Domain

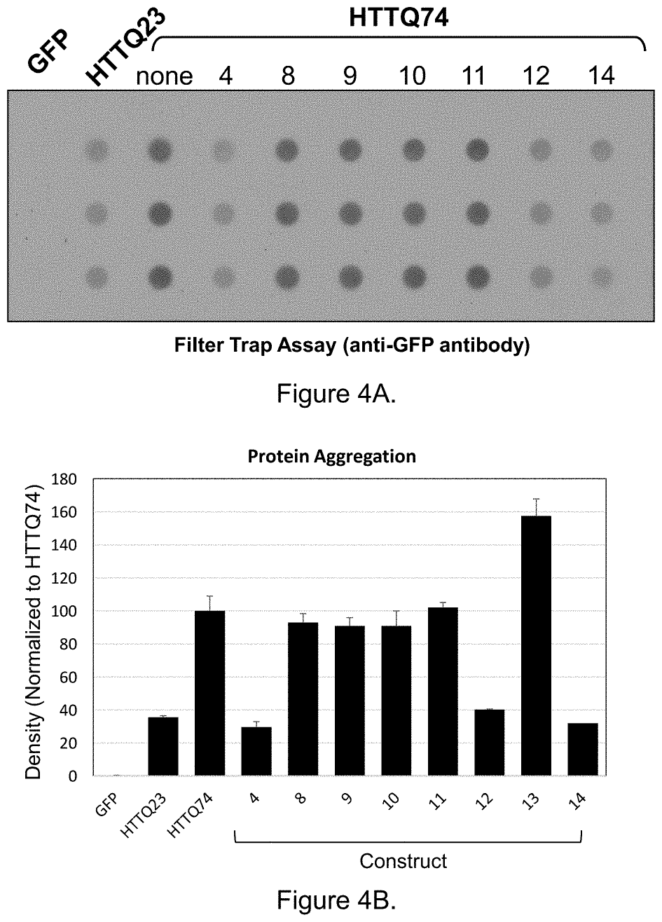

Previous experiments by the inventors employed J domain fusion proteins for the purpose of enhancing protein secretion and expression (Hishiya & Koya (2017) Sci Rep., 7:8531). In that study, it was determined that fusion protein constructs containing a fragment of a J domain as short as 11 amino acids, located within helix II, could confer enhanced secretion and overall expression of a target protein. To determine whether the requirements for a J domain in the present fusion protein constructs for reducing cytotoxicity and/or aggregation of non-secreted, cytotoxic polyglutamine repeat-containing proteins would be similar to earlier observations, constructs 8-14 were generated and tested for their ability to reduce aggregation, again using the filter trap assay and the GTP-HTTQ74 as a reporter construct. As shown in FIG. 4 and also in Table 9 below, mutating the proline in the conserved HPD motif (Construct 8) greatly diminished the ability of the construct to reduce aggregation (only 7% reduction, compared with greater than 70% reduction by the corresponding wildtype construct (Construct 4, JB1-2XQBP1).

Surprisingly, three deletion constructs containing N-terminal deletions but included the ten amino acid stretch (Constructs 9, 10 and 11) all had minimal ability to reduce aggregation (all less than 10%, compared with greater than 70% reduction by the corresponding Construct 4. Therefore, the function of the J domain when it is provided in trans appears to be significantly different than the role played by a J domain that is fused directly to the protein of interest.

TABLE 9
Relative Activity of Fusion Protein Constructs
with Altered J Domains in Reducing Aggregation

| | Fusion Protein | | |
|---|---|---|---|
| | Construct | | % Aggregation |
| Reporter | Number | Construct Name | (relative to Q74 |
| Construct | (SEQ ID NO:) | (Description) | alone) |
| GFP | None | None | 0% |
| GFP-HTTQ23 | None | None | 35.58% |
| GFP-HTTQ74 | None | None | 100% |
| GFP-HTTQ74 | 4 (92) | JB1-2XQBP1 | 29.69% |
| GFP-HTTQ74 | 8 (96) | JB1(P33Q)-2XQBP1 | 93.00% |
| GFP-HTTQ74 | 9 (97) | JB1(20-31)-2XQBP1 | 90.97% |
| | | (Part of helix II) | |
| GFP-HTTQ74 | 10 (98) | JB1(19-43)-2XQBP1 | 90.99% |
| | | (Part of helix II + | |
| | | loop + part of | |
| | | helix III) | |
| GFP-HTTQ74 | 11 (99) | JB1(17-75)-2XQBP1 | 102.12% |
| | | (Deletion of helix I) | |
| GFP-HTTQ74 | 12 (100) | JB1(1-57)-2XQBP1 | 40.26% |
| | | (Deletion of helix | |
| | | IV to C-term) | |
| GFP-HTTQ74 | 14 (102) | JB1(1-108)-2XQBP1 | 31.96% |
| | | (full J domain plus the | |
| | | glycine/phenylalanine- | |
| | | rich domain of | |
| | | DnaJB1) | |

Construct 12, which has a C-terminal truncation of the J domain, starting helix IV, was found to reduce aggregation by at least 60%. Finally, a fusion protein construct containing 33 additional amino acids from DnaJB1 (after the J domain) was found to have equivalent level of reduction in aggregation (68%) as JB1-2XQBP1 (Construct 4) (70.3%).

These results suggest that the structural requirement for J domain for reducing aggregation of the non-secreted, polyglutamine repeat-containing protein is dramatically different in the present fusion protein constructs than those tested in previous studies for enhancing protein secretion and/or expression, demonstrating that the mechanism of action of the present fusion protein constructs is distinct from those earlier studies.

iv. Effect of Linkers

Most of the fusion protein constructs described so far in this Example 1 contained a flexible linker (G₄S)₄ between the J domain and polyglutamine-binding domain. To test the importance and/or necessity of this linker, two additional constructs were generated:
Construct 15, JB1-2XQBP1 (no flexible linker), similar to JB1-2XQBP1 (Construct 4) but with the (G₄S)₄ linker removed;
Construct 16, JB1-2XQBP1 (rigid linker), also similar to JB1-2XQBP1 (Construct 4) but with the (G₄S)₄ linker replaced with a shorter, rigid linker.

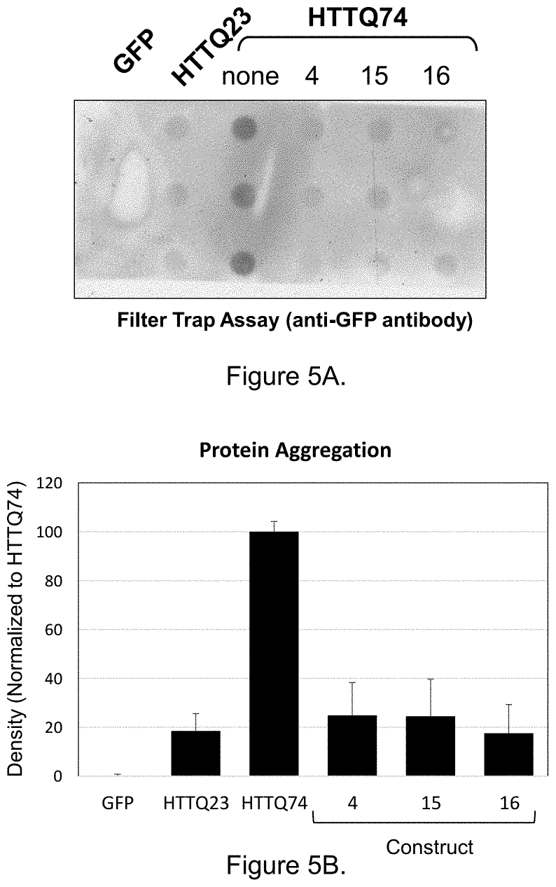

As shown in FIG. 5 and also in Table 10 below, alteration of the linker between the J domain and polyglutamine-binding domain had little effect on the ability of the fusion construct to reduce aggregation of the GFP-HTTQ74 reporter construct, with all three constructs showing at least 75% reduction.

TABLE 10
Relative Activity of Fusion Protein Constructs with Altered Linkers

| | Fusion Protein | | |
|---|---|---|---|
| | Construct | | % Aggregation |
| Reporter | Number | Construct Name | (relative to Q74 |
| Construct | (SEQ ID NO:) | (Description) | alone) |
| GFP | None | None | 0% |
| GFP-HTTQ23 | None | None | 18.49% |
| GFP-HTTQ74 | None | None | 100% |
| GFP-HTTQ74 | 4 (92) | JB1-2XQBP1 | 24.86% |
| GFP-HTTQ74 | 15 (103) | JB1-2XQBP1 | 24.50% |
| | | (no flexible linker) | |
| GFP-HTTQ74 | 16 (104) | JB1-2XQBP1 | 17.59% |
| | | (rigid linker) | |

v. Use of Different J Domains and Polyglutamine-Binding Domains

Additional constructs were generated tested to determine whether fusion protein constructs comprising other J domains are able to reduce aggregation. Table 11 below lists a number of constructs generated to test the effects of different linkers on efficacy of JB1-QBP1-QBP1 constructs:

TABLE 11
Optimization of JB1-QBP1-QBP1 constructs

| | SEQ | | | Reduction of |
|---|---|---|---|---|
| Construct | ID | Construct | | Aggregation |
| No. | NO: | Name | Description | (%) |
| 4 | 92 | JB1-2XQBP1 | JB1-long flexible-QBP1-QBP1 | 24.86% |
| 34 | 122 | JB1-QBP1-QBP1 (1) | JB1-long flexible-QBP1-Flag-QBP1 | 96.34 |
| 35 | 123 | JB1-QBP1-QBP1 (2) | JB1-QBP1-rigid-QBP1 | 97.94 |
| 36 | 124 | JB1-QBP1-QBP1 (3) | JB1-QBP1-flexible-QBP1 | 95.70 |
| 37 | 125 | JB1-QBP1-QBP1 (4) | JB1-flexible-QBP1-rigid-QBP1 | 96.95 |
| 38 | 126 | JB1-QBP1-QBP1 (5) | JB1-flexible-QBP1-flexible-QBP1 | 96.94 |
| 39 | 127 | JB1-QBP1-QBP1 (6) | JB1-rigid-QBP1-rigid-QBP1 | 98.09 |
| 40 | 128 | JB1-QBP1-QBP1 (7) | JB1-rigid-QBP1-flexible-QBP1 | 95.98 |
| 41 | 129 | JB1-QBP1-QBP1 (8) | JB1-rigid-QBP1-rigid-QBP1 | 91.07 |
| 42 | 130 | JB1-QBP1-QBP1 (9) | JB1-rigid-QBP1-flexible-QBP1 | 86.96 |
| 43 | 131 | JB1-QBP1-QBP1 (10) | JB1-QBP1-long flexible-QBP1 | 99.45 |
| 44 | 132 | JB1-QBP1-QBP1 (11) | JB1-QBP1-long rigid-QBP1 | 99.63 |
| 45 | 133 | JB1-QBP1-QBP1 (12) | JB1-flexible-QBP1-long flexible-QBP1 | 91.13 |
| 46 | 134 | JB1-QBP1-QBP1 (13) | JB1-flexible-QBP1-long rigid-QBP1 | 100 |
| 47 | 135 | JB1-QBP1-QBP1 (14) | JB1-rigid-QBP1-long flexible-QBP1 | 99.65 |
| 48 | 136 | JB1-QBP1-QBP1 (15) | JB1-rigid-QBP1-long rigid-QBP1 | 100 |
| 49 | 137 | JB1-QBP1-QBP1 (16) | JB1-long flexible-QBP1-flexible-QBP1 | 82.54 |
| 50 | 138 | JB1-QBP1-QBP1 (17) | JB1-long flexible-QBP1-rigid-QBP1 | 90.93 |
| 51 | 139 | JB1-QBP1-QBP1 (18) | JB1-QBP1-flexible-QBP1 | 96.55 |
| 52 | 140 | JB1-QBP1-QBP1 (19) | JB1-flexible-QBP1-flexible-QBP1 | 96.87 |
| 53 | 141 | Flag-QBP1-JB1-QBP1-Flag | | 79.86 |
| 54 | 142 | QBP1-JB1-QBP1 (1) | | 0 |
| 55 | 143 | QBP1-JB1-QBP1 (2) | | 9.45 |
| 56 | 144 | JB1 -JB1-QBP1 | | 68.55 |
| 57 | 145 | JB1-QBP1-JB1-QBP1 | | 94.12 |

Constructs JB1-QBP1-QBP1 (1)-JB1-QBP1-QBP1 (15) (Constructs 34-48) were co-expressed in cells along with the HTTQ74 reporter construct, and the ability to reduce protein aggregation was determined using the filter trap assay. As shown in Table 11, most constructs were found to potently reduce protein aggregation, with roughly comparable activity to Construct 4 (JB1-2XQBP1).

A number of additional constructs, provided in Table 12 below, were generated to test whether other J domain sequences, when fused with polyglutamine-binding domains, were capable of reducing protein aggregation, and compared with the efficacy of JB1-QBP1-QBP1(1) (Construct 34).

TABLE 12
Efficacy of Fusion Proteins Generated with Different J Domains

| Con- | SEQ | | | PolyQ- | # | Re- |
|---|---|---|---|---|---|---|
| struct | ID | Construct | J | binding | PolyQ | duction |
| No. | NO: | Name | Domain | Domain | Domains | (%) |
| None (mock) | | | | | | N/A |
| None (GFP-HTTQ23 reporter only) | | | | | | N/A |
| None (GFP-HTTQ74 reporter only) | | | | | | 0 |
| 34 | 122 | JB1-QBP1- | DNAJI | QBP1 | 2 | 96.1 |
| | | QBP1 (1) | | | | |
| 58 | 146 | JB6-QBP1- | DNAJB6 | QBP1 | 2 | 97.6 |
| | | QBP1 | | | | |
| 59 | 147 | JB13-QBP1- | DNAJB13 | QBP1 | 2 | −27 |
| | | QBP1 | | | | |
| 60 | 148 | JA1-QBP1- | DNAJA1 | QBP1 | 2 | 95.9 |
| | | QBP1 | | | | |
| 61 | 149 | JC7-QBP1- | DNAJC7 | QBP1 | 2 | 89.0 |
| | | QBP1 | | | | |
| 62 | 150 | DnaJ-QBPl- | Bacterial | QBP1 | 2 | 15.7 |
| | | QBP1 | DNAJ | | | |
| 63 | 151 | SV40-QBP1- | SV40 | QBP1 | 2 | 96.1 |
| | | QBP1 | DNAJ | | | |

As shown in Table 12, co-expression of Constructs 58, 60, 61 and 63 in cells expressing the HTTQ74 reporter construct all resulted in dramatically reduced protein aggregation, at a level comparable to the DNA J1-based constructs, demonstrating that multiple J domains are capable of forming active fusion proteins.

Additional constructs, including Constructs 17-31 (SEQ ID NOs: 105-119) are designed with different J domains and/or polyglutamine-binding domains as provided below in Table 13.

TABLE 13
Constructs with Alternate J Domains
and/or Polyglutamine-Binding Domains

| | SEQ | | | PolyQ- | |
|---|---|---|---|---|---|
| Construct | ID | Construct | J | binding | # PolyQ |
| No. | NO: | Name | Domain | Domain | Domains |
| 17 | 105 | JB2-2XQBP1 | DnaJB2 | QBP1 | 2 |
| 18 | 106 | JB6-2XQBP1 | DnaJB6 | QBP1 | 2 |
| 19 | 107 | JC6-2XQBP1 | DnaJC6 | QBP1 | 2 |
| 20 | 108 | JB1-2XQBP2 | DnaJB1 | QBP2 | 2 |
| 21 | 109 | JB1-scFv(MW1) | DnaJB1 | scFv(MW1) | 1 |
| 22 | 110 | JB1-polyQ(25) | DnaJB1 | polyQ(25) | 1 |
| 23 | 111 | JB2-2XQBP2 | DnaJB2 | QBP2 | 2 |
| 24 | 112 | JB6-2XQBP2 | DnaJB6 | QBP2 | 2 |
| 25 | 113 | JC6-2XQBP2 | DnaJC6 | QBP2 | 2 |
| 26 | 114 | JB2-scFv(MW1) | DnaJB2 | scFv(MW1) | 1 |
| 27 | 115 | JB6-scFv(MW1) | DnaJB6 | scFv(MW1) | 1 |
| 28 | 116 | JC6-scFv(MW1) | DnaJC6 | scFv(MW1) | 1 |
| 29 | 117 | JB2-polyQ(25) | DnaJB2 | polyQ(25) | 1 |
| 30 | 118 | JB6-polyQ(25) | DnaJB6 | polyQ(25) | 1 |
| 31 | 119 | JC6-polyQ(25) | DnaJC6 | polyQ(25) | 1 |

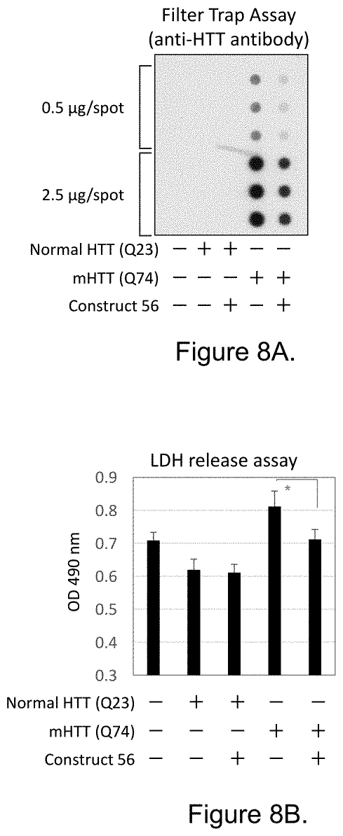

vi. Testing the reduction of aggregation-associated cytotoxicity We next tested whether, in addition to a reduction in detectable protein aggregation, we could detect reduced cytotoxicity that is often associated with protein misfolding and/or aggregation. The ability of Construct 34 (JB1-QBP1-QBP1(1), SEQ ID NO: 122) on mtHTT protein cytotoxicity was assessed in U-87 MG glioma cell line. c-myc-tagged wild type or mutant of HTT ext fragments were expressed with or without Construct 34 via lentivirus transfection. Consistent with the results above, JB1-QBP1-QBP1 (1) (Construct 34) strongly suppressed the aggregation formation (See FIG. 8A). Although mtHTT did not induce significant cytotoxicity in HEK293 cells (data not shown), cytotoxic effect caused by mtHTT (containing 74 repeats) but not in normal HTT (containing 23 repeats) was observed in U-87 MG cells, as evidenced by increased LDH activity in culture medium collected from U87-MG cells at 7-day after infection as measured by the LDH-Cytotox™ assay kit (BioLegend, San Diego, Calif., USA) (FIG. 8B) (Values represent the mean±SD. *p<0.05). Co-expression with JB1-QBP1-QBP1 (1) (Construct 34) strongly suppressed cytotoxic effect of mtHTT. These results demonstrate that the J domain fusion proteins are capable of reducing not only protein misfolding and/or aggregation, but also cytotoxicity associated with protein misfolding and/or aggregation.

vii. Testing of Constructs in Reducing Aggregation of Other Proteins

We next tested a number of constructs to determine whether it was capable of reducing aggregation of and/or eliminating other disease proteins. HEK293 cells were cultured and transfected with the plasmids encoding TDP-43 full length (FL) or TDP-43CTF (208-414) as a GFP fusion (GFP-TDP-43FL or GFP-TDP-43CTF). In some studies, c-myc-tagged TDP-43 FL or TDP-43 with a mutation in nuclear localization signal (three amino acid sequence 82KRK84 was substituted for AAA) was expressed in HEK293 cells. When Construct 53 (Flag-QBP1-JB1-QBP1-Flag, SEQ ID NO:141) was co-expressed in cells also expressing a pathogenic form of TDP-43, the total amount of aggregation-prone TDP-43 reporter was found to be greatly reduced, when compared with a companion control which had a point mutation within the conserved HPD domain (data not shown). Additional constructs comprising the DNA J domain and QBP1 were also effective in reducing pathogenic TDP-43, including JB1-QBP1 (Construct 2), JB1-2XQBP1 (Construct 4), JB1-JB1-QBP1 (Construct 56), JB1-QBP1-JB1-QBP1 (Construct 57), JB1QBP1 (1) (Construct No. 64), JB1QBP1 (2) (Construct No. 65), JB1QBP1 (3) (Construct No. 66), JB1QBP1 (4) (Construct No. 67), JB1QBP1 (5) (Construct No. 68), JB1QBP1 (6) (Construct No. 69), and JB1QBP1 (7) (Construct No. 70) (see Table 14 below):

| | SEQ | | | Reduction of |
|---|---|---|---|---|
| Construct | ID | Construct | | Aggregation |
| No. | NO: | Name | Description | (%) |
| 64 | 90 | JB1-QBP1 | JB1- long flexible- | 32.90 |
| | | | QBP1 | |
| 65 | 153 | JB1QBP1 (2) | JB1-QBP1 | 23.32 |
| 66 | 154 | JB1QBP1 (3) | JB1- flexible -QBP1 | 24.81 |
| 67 | 155 | JB1QBP1 (4) | JB1- rigid -QBP1 | 42.76 |
| 68 | 156 | JB1QBP1 (5) | JB1-QBP1 | 73.70 |
| 69 | 157 | JB1QBP1 (6) | JB1- flexible -QBP1 | 74.76 |
| 70 | 158 | JB1QBP1 (7) | JB1- rigid -QBP1 | 70.15 |

Further tests were performed to determine whether the fusion protein constructs were capable of reducing aggregation of mutagenic forms of SOD1, which are also associated with diseases such as ALS. Again, it was found that co-expression of JB1-QBP1 but not the companion control containing a point mutation in the conserved HPD domain, reduced the levels of the G85R and G93A variants of SOD1 (data not shown). The following constructs: JB1QBP1 (1) (Construct No. 64), JB1QBP1 (2) (Construct No. 65), JB1QBP1 (3) (Construct No. 66), JB1QBP1 (4) (Construct No. 67), JB1QBP1 (5) (Construct No. 68), JB1QBP1 (6) (Construct No. 69), and JB1QBP1 (7) (Construct No. 70), were also found to reduce the level of the mutagenic (G85R) variant of SOD1 (data not shown). We conclude, therefore, that the fusion proteins described herein are able to reduce pathogenic aggregation of not only polyglutamine-containing proteins, but are also useful in reducing aggregation of a number of proteins involved in protein aggregation disorders such as ALS, FTD, Parkinson's disease, Huntington's disease, Alzheimer's disease, hippocampal sclerosis, and dementia with Lewy's bodies.

### Example 2: AAV Vectors Encoding Fusion Protein Constructs

An exemplary gene therapy vector is constructed by an AAV9 vector bearing a codon-optimized cDNA encoding the fusion protein constructs of Table 5, specifically constructs 2, 4, 6, 7, 17, and 20-31, as well as control construct 1 (DnaJB1 J domain only), GFP (negative control), under the control of a CAG promoter, containing the cytomegalovirus (CMV) early enhancer element and the chicken beta-actin promoter. The cDNA encoding JB1-2XQBP1 is located downstream of the Kozak sequence and is polyadenylated by the bovine growth hormone polyadenylation (BGHpA) signal. The entire cassette is flanked by two non-coding terminal inverted sequences of AAV-2.

A nucleic acid cassette encoding Construct 53 (Flag-QBP1-JB1-QBP1-Flag, SEQ ID NO:141) driven by the strong, constitutive synthetic CAG promoter (CMV enhancer, the promoter, the first exon and the first intron of chicken beta-actin gene, and the splice acceptor of the rabbit beta-globin gene, described in Okabe et al., ibid) were placed into an AAV vector and encapsulated into the AAV rh10 capsid. A companion control construct without any insert was also encapsulated into AAV rh10.

Recombinant AAV vector is prepared using a baculovirus expression system similar to that described above (Urabe et al., 2002, Unzu et al., 2011 (reviewed in Kotin, 2011)). Briefly, three recombinant baculoviruses, one encoding REP for replication and packaging, one encoding CAP-5 for the capsid of AAV9, and one having an expression cassette is used to infect SF9 insect cells. Purification is performed using AVB Sepharose high speed affinity media (GE Healthcare Life Sciences, Piscataway, N.J.). Vectors are titrated using QPCR with the primer-probe combination for the transgene and titers are expressed as genomic copies per ml (GC/ml). The titer of the vector is approximately between 8×10¹³ to 2×10¹⁴ GC/ml.

### Example 3: Testing of Expression and Efficacy in a Mouse Model of Huntington Disease

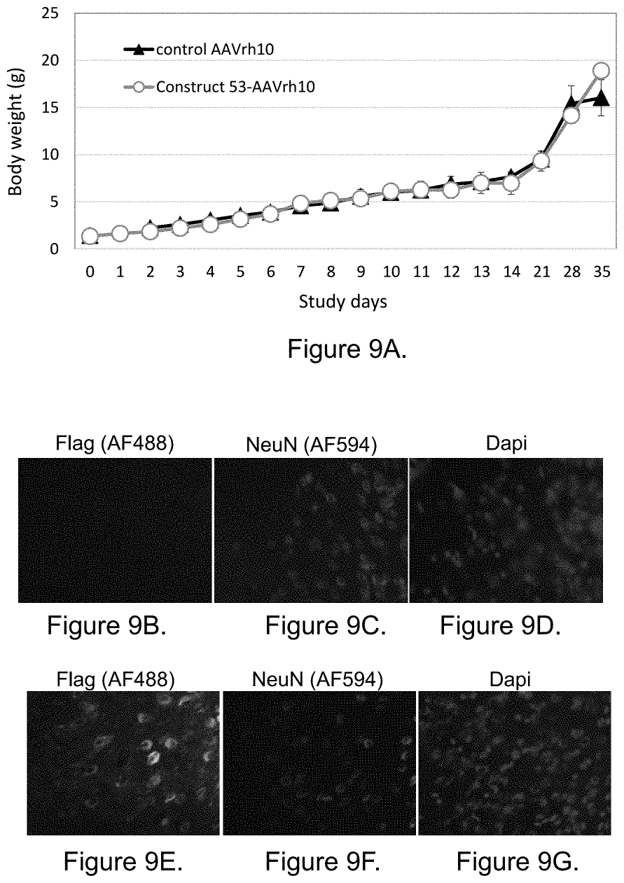

Experiments were first conducted in wild type C57BL/6J mice to confirm expression of Construct 53, and to ascertain whether expression has deleterious effects on the animal. 6×10¹⁰ vg AAV rh10 capsids containing either the control or Construct 53 as described above were injected by intracerebroventricular injection at P1. Mice were observed for ataxia, hind limb weakness, or foot dragging. One week after the AAV injection, body weights and clinical observations were made weekly. Mice (n=3) were humanely euthanized by CO₂ at 3-week to confirm the AAV expression. As shown in FIG. 9A, no difference in body weight was found during the three weeks after ICV injection. In addition, expression of the construct was confirmed by staining with anti-FLAG antibodies. As shown in FIG. 9B, frozen cortex sections were stained with anti-Flag antibody (1,4,green) and anti-NeuN antibody (2,5, red), and counterstained with Dapi (3,6, blue). Images are representative cortical areas. Panels 1-3: Injection with control AAVrh10; Panels 4-6, Injection with Construct 53 AAVrh10.

An example of such a transgenic mouse strain is the R6/2 line (Mangiarini et al., Cell 87: 493-506 (1996)). The R6/2 mice are transgenic Huntington's disease mice, which over-express exon one of the human HD gene (under the control of the endogenous promoter). The exon 1 of the R6/2 human HD gene has an expanded CAG/polyglutamine repeat lengths (150 CAG repeats on average). These mice develop a progressive, ultimately fatal neurological disease with many features of human Huntington's disease. Abnormal aggregates, constituted in part by the N-terminal part of Huntingtin (encoded by HD exon 1), are observed in R6/2 mice, both in the cytoplasm and nuclei of cells (Davies et al., Cell 90: 537-548 (1997)). For example, the human Huntingtin protein in the transgenic animal is encoded by a gene that includes at least 55 CAG repeats and more preferably about 150 CAG repeats.

These transgenic animals can develop a Huntington's disease-like phenotype, characterized by reduced weight gain, reduced lifespan and motor impairment characterized by abnormal gait, resting tremor, hindlimb clasping and hyperactivity from 8 to 10 weeks after birth (see, for example Mangiarini et al., ibid). The phenotype progressively worsens toward hypokinesia. The brains of these transgenic mice also demonstrate neurochemical and histological abnormalities, such as changes in neurotransmitter receptors (glutamate, dopaminergic), decreased concentration of N-acetylaspartate (a marker of neuronal integrity) and reduced striatum and brain size. Accordingly, evaluating can include assessing parameters related to neurotransmitter levels, neurotransmitter receptor levels, brain size and striatum size. In addition, abnormal aggregates containing the transgenic part of or full-length human Huntingtin protein are present in the brain tissue of these animals (e.g., the R6/2 transgenic mouse strain). See, e.g., Mangiarini et al., ibid, Davies et al., Cell 90: 537-548 (1997), Brouillet, Functional Neurology 15(4): 239-251 (2000) and Cha et al., Proc. Natl. Acad. Sci. USA 95: 6480-6485 (1998).

To test the effect of the test compound or known compound described in the application in an animal model, the different AAV viral particles containing vectors encoding the fusion proteins and corresponding controls are administered to the transgenic animal. In one embodiment, the viral particles are administered by tail vein injection. In another embodiment, the viral particles are administered by intramuscular injection. In still another embodiment, the particles are administered by intracranial injection, for example as described in Stanek et al., (2014) Hum. Gene. Ther. 25:461-474.

After administration, disease progression is monitored and compared with control injected mice. In one embodiment, a Huntington's disease-like symptom is evaluated in the animal. For example, the progression of the Huntington's disease-like symptoms, e.g., as described above for the mouse model, is then monitored to determine whether treatment with the test compound results in reduction or delay of symptoms. In another embodiment, disaggregation of the Huntingtin protein aggregates in these animals is monitored. The animal can then be sacrificed and brain slices are obtained. The brain slices are then analyzed for the presence of aggregates containing the transgenic human Huntingtin protein, a portion thereof, or a fusion protein comprising human Huntingtin protein, or a portion thereof. This analysis can includes, for example, staining the slices of brain tissue with anti-Huntingtin antibody and adding a secondary antibody conjugated with FITC which recognizes the anti-Huntingtin's antibody (for example, the anti-Huntingtin antibody is mouse anti-human antibody and the secondary antibody is specific for human antibody) and visualizing the protein aggregates by fluorescent microscopy. Alternatively, the anti-Huntingtin antibody can be directly conjugated with FITC (see, for example, Shinkawa et al., (2011) Mol. Biol. Cell, 22:3571-3583, which is incorporated herein by reference in its entirety). The levels of Huntingtin's protein aggregates are then visualized by fluorescent microscopy.

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

What is claimed is:

1. An isolated fusion protein comprising a J domain of a J protein and a polyglutamine-binding domain.

2. The fusion protein of claim 1, wherein the J domain of a J protein is of eukaryotic origin.

3. The fusion protein of claim 1 or claim 2, wherein the J domain of a J protein is of human origin.

4. The fusion protein of any one of claims 1-3, wherein the J domain of a J protein is cytosolically localized.

5. The fusion protein of any one of claims 1-4, wherein the J domain of a J protein is selected from the group consisting of SEQ ID Nos: 1-55.

6. The fusion protein of any one of claims 1-5, wherein the J domain comprises the sequence selected from the group consisting of SEQ ID NOs: 1, 5, 6, 10, 16, 24, 25, 31 and 49.

7. The fusion protein of any one of claims 1-6, wherein the J domain comprises the sequence of SEQ ID NO: 5.

8. The fusion protein of any one of claims 1-6, wherein the J domain comprises the sequence of SEQ ID NO: 10.

9. The fusion protein of any one of claims 1-6, wherein the J domain comprises the sequence of SEQ ID NO: 24.

10. The fusion protein of any one of claims 1-6, wherein the J domain comprises the sequence of SEQ ID NO: 31.

11. The fusion protein of any one of claims 1-6, wherein the J domain comprises the sequence of SEQ ID NO: 49.

12. The fusion protein of any one of claims 1-11, wherein the polyglutamine-binding domain comprises the sequence selected from the group consisting of SEQ ID NOs: 51-68.

13. The fusion protein of any one of claims 1-12, wherein the polyglutamine-binding domain comprises the sequence of SEQ ID NO:57.

14. The fusion protein of any one of claims 1-13, comprising a plurality of polyglutamine-binding domains.

15. The fusion protein of any one of claims 1-14, consisting of two polyglutamine-binding domains.

16. The fusion protein of any one of claims 1-15, comprising one of the following constructs:
a. DNAJ-X-Q,
b. DNAJ-X-Q-X-Q,
c. DNAJ-X-Q-X-Q-X-Q,
d. Q-X-DNAJ,
e. Q-X-Q-X-DNAJ,
f. Q-X-Q-X-Q-X-DNAJ,
g. Q-X-DNAJ-X-Q,
h. Q-X-DNAJ-X-Q-X-Q,
i. DNAJ-X-DNAJ-X-Q,
j. Q-X-Q-X-DNAJ-X-Q,
k. DNAJ-X-Q-X-DNAJ-X-Q,
l. Q-X-Q-X-DNAJ-X-Q-X-Q-X-Q,
m. Q-X-Q-X-Q-X-DNAJ-X-Q,
n. Q-X-Q-X-Q-X-DNAJ-X-Q-X-Q,
o. Q-X-Q-X-Q-X-DNAJ-X-Q-X-Q-X-Q,
p. DnaJ-X-DnaJ-X-Q-X-Q,
q. Q-X-DnaJ-X-DnaJ,
r. Q-X-Q-X-DnaJ-X-DnaJ, and
s. Q-X-DnaJ-X-DnaJ-X-Q
wherein,
Q is a polyglutamine-binding domain,
DNAJ is a J domain of a J protein, and
X is an optional linker.

17. The fusion protein of any one of claims 1-16, wherein the fusion protein comprises the J domain sequence of SEQ ID NO: 5 and the polyglutamine-binding domain sequence of SEQ ID NO: 57.

18. The fusion protein of any one of claims 1-17, wherein the fusion protein comprises the J domain sequence of SEQ ID NO: 5 and two copies of the polyglutamine-binding domain sequence of SEQ ID NO: 57.

19. The fusion protein of any one of claims 1-18, wherein the fusion protein comprises the sequence selected from the group consisting of SEQ ID Nos: 89-157.

20. The fusion protein of any one of claims 1-19, wherein the fusion protein comprises the sequence of SEQ ID NO: 90.

21. The fusion protein of any one of claims 1-19, wherein the fusion protein comprises the sequence of SEQ ID NO: 91.

22. The fusion protein of any one of claims 1-19, wherein the fusion protein comprises the sequence of SEQ ID NO: 92.

23. The fusion protein of any one of claims 1-19, wherein the fusion protein comprises the sequence of SEQ ID NO: 93.

24. The fusion protein of any one of claims 1-23, further comprising a targeting reagent.

25. The fusion protein of any one of claims 1-24, further comprising an epitope.

26. The fusion protein of any one of claims 1-25, further comprising a signal sequence.

27. The fusion protein of any one of claims 1-26, which is capable of reducing aggregation of polyglutamine-containing proteins in a cell.

28. The fusion protein of any one of claims 1-27, which is capable of reducing polyglutamine repeat-mediated cytotoxicity.

29. A nucleic acid sequence encoding the fusion protein of any one of claims 1-28.

30. The nucleic acid sequence of claim 29, wherein said nucleic acid is DNA.

31. The nucleic acid sequence of claim 29 or claim 30, wherein said nucleic acid comprises at least one modified nucleic acid.

32. The nucleic acid sequence of any one of claims 29-31, further comprising a promoter region, 5′ UTR, 3′ UTR and a poly(A) signal.

33. The nucleic acid sequence of claim 32, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.

34. A vector comprising the nucleic acid sequence of any one of claims 29-33.

35. The vector of claim 34, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.

36. The vector of claim 34 or claim 35, wherein the vector is an AAV.

37. A virus particle comprising a capsid and the vector of any one of claims 34-36.

38. The virus particle of claim 37, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.

39. The virus particle of claim 37 or claim 38, wherein the capsid is selected from the group consisting of AAV2, AAV5, AAV8, AAV9 and AAVrh10.

40. The virus particle of any one of claims 37-39, wherein the capsid is AAV2.

41. The virus particle of any one of claims 37-39, wherein the capsid is AAV5.

42. The virus particle of any one of claims 37-39, wherein the capsid is AAV8.

43. The virus particle of any one of claims 37-39, wherein the capsid is AAV9.

44. The virus particle of any one of claims 37-39, wherein the capsid is AAV rh10.

45. A pharmaceutical composition comprising an agent selected from the group consisting of the fusion protein of any one of claims 1-28, a cell expressing the fusion protein of any one of claims 1-28, the nucleic acid of any one of claims 29-33, the vector of any one of claims 34-36, the virus particle of any one of claims 37-44, and a pharmaceutically acceptable carrier or excipient.

46. A method of reducing toxicity of a polyglutamine protein in a cell, comprising contacting said cell with an effective amount of one or more agents selected from the group consisting of: fusion protein of any one of claims 1-28, a cell expressing the fusion protein of any one of claims 1-28, the nucleic acid of any one of claims 29-33, the vector of any one of claims 34-36, the virus particle of any one of claims 37-44 and the pharmaceutically composition of claim 45.

47. The method of claim 46, wherein the cell is in a subject.

48. The method of claim 46 or claim 47, wherein the subject is a human.

49. The method of any one of claims 46-48, wherein the cell is a cell of the central nervous system.

50. The method of any one of claims 46-49, wherein subject is identified as having a polyglutamine repeat disease.

51. The method of any one of claims 46-50, wherein the polyglutamine protein is selected from the group consisting of huntingtin, atrophin-1, ataxin 1, ataxin 2, Cav2.1, ataxin 7, TATA-binding protein, ataxin 3, and androgen receptor.

52. The method of any one of claims 46-51, wherein there is a reduction in aggregation of the polyglutamine protein in the cell.

53. A method of treating, preventing, or delaying the progression of a polyglutamine repeat disease in a subject in need thereof, the method comprising administering an effective amount of one or more agents selected from the group consisting of fusion protein of any one of claims 1-28, a cell expressing the fusion protein of any one of claims 1-28, the nucleic acid of any one of claims 29-33, the vector of any one of claims 34-36, the virus particle of any one of claims 37-44 and the pharmaceutically composition of claim 45.

54. The method of claim 53, wherein the polyglutamine repeat disease is selected from the group consisting of Huntington's disease, SCA type 1, SCA type 2, SCA type 6, SCA type 7, SCA type 17, MJD/SCA3, DRPLA, and SBMA.

55. Use of one or more of the fusion protein of any one of claims 1-28, a cell expressing the fusion protein of any one of claims 1-28, the nucleic acid of any one of claims 29-33, the vector of any one of claims 34-36, the virus particle of any one of claims 37-44 and the pharmaceutically composition of claim 45 for use in preventing or delaying the progression of a polyglutamine repeat disease in a subject.

56. A method of reducing protein aggregation in a cell, comprising contacting said cell with an effective amount of one or more agents selected from the group consisting of fusion protein of any one of claims 1-28, a cell expressing the fusion protein of any one of claims 1-28, the nucleic acid of any one of claims 29-33, the vector of any one of claims 34-36, the virus particle of any one of claims 37-44 and the pharmaceutically composition of claim 45.

57. The method of claim 56, wherein the cell is in a subject.

58. The method of claim 56 or claim 57, wherein the subject is a human.

59. The method of any one of claims 56-58, wherein the cell is a cell of the central nervous system.

60. The method of any one of claims 56-59, wherein subject is identified as having a polyglutamine repeat disease.

61. The method of any one of claims 56-60, wherein the polyglutamine protein is selected from the group consisting of huntingtin, atrophin-1, ataxin 1, ataxin 2, Cav2.1, ataxin 7, TATA-binding protein, ataxin 3, and androgen receptor.

62. The method of any one of claims 56-61, wherein subject is identified as having a disease selected from the group consisting of ALS, FTD, Parkinson's disease, Huntington's disease, Alzheimer's disease, hippocampal sclerosis, and dementia with Lewy's bodies.

63. The method of any one of claims 56-62, wherein there is a reduction in aggregation of the protein in the cell.

64. A method of treating, preventing, or delaying the progression of a protein aggregation disease in a subject in need thereof, the method comprising administering an effective amount of one or more agents selected from the group consisting of fusion protein of any one of claims 1-28, a cell expressing the fusion protein of any one of claims 1-28, the nucleic acid of any one of claims 29-33, the vector of any one of claims 34-36, the virus particle of any one of claims 37-44 and the pharmaceutically composition of claim 45.

65. The method of claim 64, wherein the polyglutamine repeat disease is selected from the group consisting of Huntington's disease, SCA type 1, SCA type 2, SCA type 6, SCA type 7, SCA type 17, MJD/SCA3, DRPLA, and SBMA.

66. Use of one or more of the fusion protein of any one of claims 1-28, a cell expressing the fusion protein of any one of claims 1-28, the nucleic acid of any one of claims 29-33, the vector of any one of claims 34-36, the virus particle of any one of claims 37-44 and the pharmaceutically composition of claim 45 for use in preventing or delaying the progression of a protein aggregation disease in a subject.


SEQUENCE LISTING (170 sequences)

SEQ ID NO: 1 - protein, 63 aa, Artificial
  DNAJA1
TYYDVLGVKPNATQEELKKAYRKLALKYHPDKNPNEGEKFKQISQAYEVLSDAKKRELYDKGG

SEQ ID NO: 2 - protein, 63 aa, Artificial
  DNAJA2
KLYDILGVPPGASENELKKAYRKLAKEYHPDKNPNAGDKFKEISFAYEVLSNPEKRELYDRYG

SEQ ID NO: 3 - protein, 66 aa, Artificial
  DNAJA3
DYYQILGVPRNASQKEIKKAYYQLAKKYHPDTNKDDPKAKEKFSQLAEAYEVLSDEVKRKQYDAYG

SEQ ID NO: 4 - protein, 67 aa, Artificial
  DNAJA4
ETQYYDILGVKPSASPEEIKKAYRKLALKYHPDKNPDEGEKFKLISQAYEVLSDPKKRDVYDQGGEQ

SEQ ID NO: 5 - protein, 69 aa, Artificial
  DNAJB1
GKDYYQTLGLARGASDEEIKRAYRRQALRYHPDKNKEPGAEEKFKEIAEAYDVLSDPRKREIFDRYGEE

SEQ ID NO: 6 - protein, 70 aa, Artificial
  DNAJB2
ASYYEILDVPRSASADDIKKAYRRKALQWHPDKNPDNKEFAEKKFKEVAEAYEVLSDKHKREIYDRYGRE

SEQ ID NO: 7 - protein, 69 aa, Artificial
  DNAJB3
MVDYYEVLDVPRQASSEAIKKAYRKLALKWHPDKNPENKEEAERRFKQVAEAYEVLSDAKKRDIYDRYG

SEQ ID NO: 8 - protein, 69 aa, Artificial
  DNAJB4
GKDYYCILGIEKGASDEDIKKAYRKQALKFHPDKNKSPQAEEKFKEVAEAYEVLSDPKKREIYDQFGEE

SEQ ID NO: 9 - protein, 65 aa, Artificial
  DNAJB5
DYYKILGIPSGANEDEIKKAYRKMALKYHPDKNKEPNAEEKFKEIAEAYDVLSDPKKRGLYDQYG

SEQ ID NO: 10 - protein, 68 aa, Artificial
  DNAJB6
VDYYEVLGVQRHASPEDIKKAYRKLALKWHPDKNPENKEEAERKFKQVAEAYEVLSDAKKRDIYDKYG

SEQ ID NO: 11 - protein, 67 aa, Artificial
  DNAJB7
DYYEVLGLQRYASPEDIKKAYHKVALKWHPDKNPENKEEAERKFKEVAEAYEVLSNDEKRDIYDKYG

SEQ ID NO: 12 - protein, 67 aa, Artificial
  DNAJB8
NYYEVLGVQASASPEDIKKAYRKLALRWHPDKNPDNKEEAEKKFKLVSEAYEVLSDSKKRSLYDRAG

SEQ ID NO: 13 - protein, 65 aa, Artificial
  DNAJB9
SYYDILGVPKSASERQIKKAFHKLAMKYHPDKNKSPDAEAKFREIAEAYETLSDANRRKEYDTLG

SEQ ID NO: 14 - protein, 66 aa, Artificial
  DNAJB11
DFYKILGVPRSASIKDIKKAYRKLALQLHPDRNPDDPQAQEKFQDLGAAYEVLSDSEKRKQYDTYG

SEQ ID NO: 15 - protein, 65 aa, Artificial
  DNAJB12
YEILGVSRGASDEDLKKAYRRLALKFHPDKNHAPGATEAFKAIGTAYAVLSNPEKRKQYDQFGDD

SEQ ID NO: 16 - protein, 65 aa, Artificial
  DNAJB13
DYYSVLGITRNSEDAQIKQAYRRLALKHHPLKSNEPSSAEIFRQIAEAYDVLSDPMKRGIYDKFG

SEQ ID NO: 17 - protein, 65 aa, Artificial
  DNAJB14
NYYEVLGVTKDAGDEDLKKAYRKLALKFHPDKNHAPGATDAFKKIGNAYAVLSNPEKRKQYDLTG

SEQ ID NO: 18 - protein, 65 aa, Artificial
  DNAJC1
NFYQFLGVQQDASSADIRKAYRKLSLTLHPDKNKDENAETQFRQLVAIYEVLKDDERRQRYDDIL

SEQ ID NO: 19 - protein, 74 aa, Artificial
  DNAJC2
DHYAVLGLGHVRYKATQRQIKAAHKAMVLKHHPDKRKAAGEPIKEGDNDYFTCITKAYEMLSDPVKRRAFNSVD

SEQ ID NO: 20 - protein, 69 aa, Artificial
  DNAJC3
DYYKILGVKRNAKKQEIIKAYRKLALQWHPDNFQNEEEKKKAEKKFIDIAAAKEVLSDPEMRKKFDDGE

SEQ ID NO: 21 - protein, 66 aa, Artificial
  DNAJC4
TYYELLGVHPGASTEEVKRAFFSKSKELHPDRDPGNPSLHSRFVELSEAYRVLSREQSRRSYDDQL

SEQ ID NO: 22 - protein, 70 aa, Artificial
  DNAJC5
GESLYHVLGLDKNATSDDIKKSYRKLALKYHPDKNPDNPEAADKFKEINNAHAILTDATKRNIYDKYGSL

SEQ ID NO: 23 - protein, 66 aa, Artificial
  DNAJC5B
ALYEILGLHKGASNEEIKKTYRKLALKHHPDKNPDDPAATEKFKEINNAHAILTDISKRSIYDKYG

SEQ ID NO: 24 - protein, 65 aa, Artificial
  DNAJC6
TKWKPVGMADLVTPEQVKKVYRKAVLVVHPDKATGQPYEQYAKMIFMELNDAWSEFENQGQKPLY

SEQ ID NO: 25 - protein, 71 aa, Artificial
  DNAJC7
DYYKILGVDKNASEDEIKKAYRKRALMHHPDRHSGASAEVQKEEEKKFKEVGEAFTILSDPKKKTRYDSGQ

SEQ ID NO: 26 - protein, 68 aa, Artificial
  DNAJC8
NPFEVLQIDPEVTDEEIKKRFRQLSILVHPDKNQDDADRAQKAFEAVDKAYKLLLDQEQKKRALDVIQ

SEQ ID NO: 27 - protein, 68 aa, Artificial
  DNAJC9
DLYRVLGVRREASDGEVRRGYHKVSLQVHPDRVGEGDKEDATRRFQILGKVYSVLSDREQRAVYDEQG

SEQ ID NO: 28 - protein, 66 aa, Artificial
  DNAJC10
DFYSLLGVSKTASSREIRQAFKKLALKLHPDKNPNNPNAHGDFLKINRAYEVLKDEDLRKKYDKYG

SEQ ID NO: 29 - protein, 69 aa, Artificial
  DNAJC11
DYYSLLNVRREASSEELKAAYRRLCMLYHPDKHRDPELKSQAERLFNLVHQAYEVLSDPQTRAIYDIYG

SEQ ID NO: 30 - protein, 66 aa, Artificial
  DNAJC12
DYYTLLGCDELSSVEQILAEFKVRALECHPDKHPENPKAVETFQKLQKAKEILTNEESRARYDHWR

SEQ ID NO: 31 - protein, 66 aa, Artificial
  DNAJC13
DAYEVLNLPQGQGPHDESKIRKAYFRLAQKYHPDKNPEGRDMFEKVNKAYEFLCTKSAKIVDGPDP

SEQ ID NO: 32 - protein, 65 aa, Artificial
  DNAJC14
NPFHVLGVEATASDVELKKAYRQLAVMVHPDKNHHPRAEEAFKVLRAAWDIVSNAEKRKEYEMKR

SEQ ID NO: 33 - protein, 55 aa, Artificial
  DNAJC15
EAGLILGVSPSAGKAKIRTAHRRVMILNHPDKGGSPYVAAKINEAKDLLETTTKH

SEQ ID NO: 34 - protein, 65 aa, Artificial
  DNAJC16
DPYRVLGVSRTASQADIKKAYKKLAREWHPDKNKDPGAEDKFIQISKAYEILSNEEKRSNYDQYG

SEQ ID NO: 35 - protein, 66 aa, Artificial
  DNAJC17
DLYALLGIEEKAADKEVKKAYRQKALSCHPDKNPDNPRAAELFHQLSQALEVLTDAAARAAYDKVR

SEQ ID NO: 36 - protein, 65 aa, Artificial
  DNAJC18
NYYEILGVSRDASDEELKKAYRKLALKFHPDKNCAPGATDAFKAIGNAFAVLSNPDKRLRYDEYG

SEQ ID NO: 37 - protein, 55 aa, Artificial
  DNAJC19
EAALILGVSPTANKGKIRDAHRRIMLLNHPDKGGSPYIAAKINEAKDLLEGQAKK

SEQ ID NO: 38 - protein, 72 aa, Artificial
  DNAJC20
DYFSLMDCNRSFRVDTAKLQHRYQQLQRLVHPDFFSQRSQTEKDFSEKHSTLVNDAYKTLLAPLSRGLYLLK

SEQ ID NO: 39 - protein, 67 aa, Artificial
  DNAJC21
CHYEALGVRRDASEEELKKAYRKLALKWHPDKNLDNAAEAAEQFKLIQAAYDVLSDPQERAWYDNHR

SEQ ID NO: 40 - protein, 65 aa, Artificial
  DNAJC22
LAYQVLGLSEGATNEEIHRSYQELVKVWHPDHNLDQTEEAQRHFLEIQAAYEVLSQPRKPWGSRR

SEQ ID NO: 41 - protein, 62 aa, Artificial
  DNAJC23
NPYEVLNLDPGATVAEIKKQYRLLSLKYHPDKGGDEVMFMRIAKAYAALTDEESRKNWEEFG

SEQ ID NO: 42 - protein, 72 aa, Artificial
  DNAJC24
DWYSILGADPSANISDLKQKYQKLILMYHPDKQSTDVPAGTVEECVQKFIEIDQAWKILGNEETKREYDLQR

SEQ ID NO: 43 - protein, 76 aa, Artificial
  DNAJC25
DCYEVLGVSRSAGKAEIARAYRQLARRYHPDRYRPQPGDEGPGRTPQSAEEAFLLVATAYETLKDEETRKDYDYML

SEQ ID NO: 44 - protein, 65 aa, Artificial
  DNAJC26
SRWTPVGMADLVAPEQVKKHYRRAVLAVHPDKAAGQPYEQHAKMIFMELNDAWSEFENQGSRPLF

SEQ ID NO: 45 - protein, 57 aa, Artificial
  DNAJC27
DSWDMLGVKPGASRDEVNKAYRKLAVLLHPDKCVAPGSEDAFKAVVNARTALLKNIK

SEQ ID NO: 46 - protein, 65 aa, Artificial
  DNAJC28
EYYRLLNVEEGCSADEVRESFHKLAKQYHPDSGSNTADSATFIRIEKAYRKVLSHVIEQTNASQS

SEQ ID NO: 47 - protein, 88 aa, Artificial
  DNAJC29
ILKEVTSVVEQAWKLPESERKKIIRRLYLKWHPDKNPENHDIANEVFKHLQNEINRLEKQAFLDQNADRASRRTFSTSASRFQSDKYS

SEQ ID NO: 48 - protein, 66 aa, Artificial
  DNAJC30
ALYDLLGVPSTATQAQIKAAYYRQCFLYHPDRNSGSAEAAERFTRISQAYVVLGSATLRRKYDRGL

SEQ ID NO: 49 - protein, 64 aa, Artificial
  SV40 Jdomain
QLMDLLGLERSAWGNIPLMRKAYLKKCKEFHPDKGGDEEKMKKMNTLYKKMEDGVKYAHQPDFG

SEQ ID NO: 50 - protein, 70 aa, Artificial
  Bacterial J-domain
KQDYYEILGVSKTAEEREIRKAYKRLAMKYHPDRNQGDKEAEAKFKEIKEAYEVLTDSQKRAAYDQYGHA

SEQ ID NO: 51 - protein, 265 aa, Artificial
  PQBP1
MPLPVALQTRLAKRGILKHLEPEPEEEIIAEDYDDDPVDYEATRLEGLPPSWYKVFDPSCGLPYYWNADTDLVSWLSPHDPNSVVTKSAKKLRSSNADAEEKLDRSHDKSDRGHDKSDRSHEKLDRGHDKSDRGHDKSDRDRERGYDKVDRERERDRERDRDRGYDKADREEGKERRHHRREELAPYPKSKKAVSRKDEELDPMDPSSYSDAPRGTWSTGLPKRNEAKTGADTTAAGPLFQQRPYPSPGAVLRANAEASRTKQQD

SEQ ID NO: 52 - protein, 60 aa, Artificial
  PQBP-2
RPDQERLLLRGWVPRWPHQPAAAEAAPDRVPPELTLTLQYSRNTERCGIHMAIHHCQPDN

SEQ ID NO: 53 - protein, 46 aa, Artificial
  PQBP-3
RRRWWTRASWPRRRRRRGTGCCSGSPTAARPPSPWRKTRKGTMSLT

SEQ ID NO: 54 - protein, 24 aa, Artificial
  PQBP-4
RRGTRRLKMNRLQPFITHGYLCHE

SEQ ID NO: 55 - protein, 41 aa, Artificial
  PQBP-5
RRRKSRKENQVMQKVRRVQMMKKPGLKRSGSNADSSSRRKK

SEQ ID NO: 56 - protein, 35 aa, Artificial
  VCP
RFPSGNQGGAGPSQGSGSGTGGSVYTEDNDDDLYG

SEQ ID NO: 57 - protein, 11 aa, Artificial
  QBP1
SNWKWWPGIFD

SEQ ID NO: 58 - protein, 11 aa, Artificial
  QBP2
HWWRSWYSDSV

SEQ ID NO: 59 - protein, 11 aa, Artificial
  QBP3
HEWHWWHQEAA

SEQ ID NO: 60 - protein, 11 aa, Artificial
  QBP4
WGLEHFAGNKR

SEQ ID NO: 61 - protein, 11 aa, Artificial
  QBP5
WWRWNWATPVD

SEQ ID NO: 62 - protein, 11 aa, Artificial
  QBP6
WHNYFHWWQDT

SEQ ID NO: 63 - protein, 116 aa, Artificial
  MW1-LC
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 64 - protein, 118 aa, Artificial
  MW1-HC
QVQLQESGGGLVQPGGSLKLSCAASGFTFRDYYMYWVRQTPEKRLEWVAFISNGGGSTYYPDTVKGRFTISRDNAKNTLYLQMSRLKSEDTAMYYCARGRGYVWFAYWGQGTTVTVSS

SEQ ID NO: 65 - protein, 116 aa, Artificial
  1C2-LC
QLVLTQSSSASFSLGASAKLTCTLSRQHSTYTIEWYQQQPLKPPKFVMELKKDGSHSTGDGIPDRFSGSSSGAHRYLSISNIQPEDEAIYICGVGDTIKEQFVYVFGGGTKVTVLG

SEQ ID NO: 66 - protein, 25 aa, Artificial
  PolyQ(25)
QQQQQQQQQQQQQQQQQQQQQQQQQ

SEQ ID NO: 67 - protein, 243 aa, Artificial
  MW7
QVKLQESGGGLVQPGGSMKLSCAASGFTFSDAWMDWVRQSPEKGLSGVAEIRSKANNHATYYAESVKGRFTISRDDSKSSVYLQMNSLRAEDTGIYYCIYAGFAYWGQGTTVTVSSGGGGSGGGGSGGGGSDIELTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPWTFGGGTKLEI

SEQ ID NO: 68 - protein, 118 aa, Artificial
  Happ1
MQSVLTQPPSASGTPGQRVTISCSGSSSNIGSNYVYWYQQLPGTAPKLLIYRNNQRPSGVPDRFSGSKSGTSASLAISGLRPEDEADYYCAAWDDSLCVALVFGGGTNGGGGVDGTAG

SEQ ID NO: 69 - skipped

SEQ ID NO: 70 - protein, 4 aa, Artificial
  GTGS
GTGS

SEQ ID NO: 71 - protein, 5 aa, Artificial
  GLESR
GLESR

SEQ ID NO: 72 - protein, 4 aa, Artificial
  GGSG
GGSG

SEQ ID NO: 73 - protein, 4 aa, Artificial
  GGGS
GGGS

SEQ ID NO: 74 - protein, 5 aa, Artificial
  DIAAA
DIAAA

SEQ ID NO: 75 - protein, 9 aa, Artificial
  DIAAALESR
DIAAALESR

SEQ ID NO: 76 - protein, 15 aa, Artificial
  GGGGSGGGGSGGGGS
GGGGSGGGGSGGGGS

SEQ ID NO: 77 - protein, 11 aa, Artificial
  AEAAAKEAAAK
AEAAAKEAAAK

SEQ ID NO: 78 - protein, 15 aa, Artificial
  SGGGSGGGGSGGGGS
SGGGSGGGGSGGGGS

SEQ ID NO: 79 - protein, 25 aa, Artificial
  DIGGGGSGGGGSGGGGSGGGGSAAA
DIGGGGSGGGGSGGGGSGGGGSAAA

SEQ ID NO: 80 - protein, 5 aa, Artificial
  GGGGS
GGGGS

SEQ ID NO: 81 - protein, 5 aa, Artificial
  EAAAK
EAAAK

SEQ ID NO: 82 - protein, 10 aa, Artificial
  GGGGSGGGGS
GGGGSGGGGS

SEQ ID NO: 83 - protein, 10 aa, Artificial
  EAAAKEAAAK
EAAAKEAAAK

SEQ ID NO: 84 - skipped

SEQ ID NO: 85 - protein, 9 aa, Artificial
  RKKRRQRRR
RKKRRQRRR

SEQ ID NO: 86 - protein, 15 aa, Artificial
  RQIKWFQNRRMKWKK
RQIKWFQNRRMKWKK

SEQ ID NO: 87 - protein, 21 aa, Artificial
  KETWWETWWTEWSQPKKKRKV
KETWWETWWTEWSQPKKKRKV

SEQ ID NO: 88 - protein, 17 aa, Artificial
  CSIPPEVKFNKPFVYLI
CSIPPEVKFNKPFVYLI

SEQ ID NO: 89 - protein, 92 aa, Artificial
  JB1
MGKDYYQTLGLARGASDEEIKRAYRRQALRYHPDKNKEPGAEEKFKEIAEAYDVLSDPRKREIFDRYGEEGLKGSDIAAALESRDYKDDDDK

SEQ ID NO: 90 - protein, 114 aa, Artificial
  JB1-QBP1
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFDGLE

SEQ ID NO: 91 - protein, 114 aa, Artificial
  QBP1-JB1
MGKPIPNPLLGLDSTGTGSSNWKWWPGIFDEFMGKDYYQTLGLARGASDEEIKRAYRRQALRYHPDKNKEPGAEEKFKEIAEAYDVLSDPRKREIFDRYGEEGLKGSDIAAALE

SEQ ID NO: 92 - protein, 125 aa, Artificial
  JB1-2XQBP1
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFDGLESNWKWWPGIFD

SEQ ID NO: 93 - protein, 131 aa, Artificial
  JB1-3XQBP1
MGKDYYQTLGLARGASDEEIKRAYRRQALRYHPDKNKEPGAEEKFKEIAEAYDVLSDPRKREIFDRYGEEGLKGSGGGGSGGGGSGGGGSGGGGSGSSNWKWWPGIAAASNWKWWPGILESNWKWWPGIFD

SEQ ID NO: 94 - protein, 146 aa, Artificial
  QBP1-JB1-QBP1
MGKPIPNPLLGLDSTGTGSSNWKWWPGIFDEFMGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFDGLE

SEQ ID NO: 95 - protein, 157 aa, Artificial
  QBP1-JB1-2XQBP1
MGKPIPNPLLGLDSTGTGSSNWKWWPGIFDEFMGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFDGLESNWKWWPGIFD

SEQ ID NO: 96 - protein, 125 aa, Artificial
  JB1(P33Q)-2XQBP1
MGKDYYQTLGLARGASDEEIKRAYRRQALRYHQDKNKEPGAEEKFKEIAEAYDVLSDPRKREIFDRYGEEGLKGSDIGGGGSGGGGSGGGGSGGGGSAAASNWKWWPGIFDGLESNWKWWPGIFD

SEQ ID NO: 97 - protein, 69 aa, Artificial
  JB1(20-31)-2XQBP1
MGTGSEFIKRAYRRQALRYDIGGGGSGGGGSGGGGSGGGGSAAASNWKWWPGIFDGLESNWKWWPGIFD

SEQ ID NO: 98 - protein, 82 aa, Artificial
  JB1(19-43)-2XQBP1
MGTGSEFEIKRAYRRQALRYHPDKNKEPGAEEDIGGGGSGGGGSGGGGSGGGGSAAASNWKWWPGIFDGLESNWKWWPGIFD

SEQ ID NO: 99 - protein, 116 aa, Artificial
  JB1(17-75)-2XQBP1
MGTGSEFDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFDGLESNWKWWPGIFD

SEQ ID NO: 100 - protein, 107 aa, Artificial
  JB1(1-57)-2XQBP1
MGKDYYQTLGLARGASDEEIKRAYRRQALRYHPDKNKEPGAEEKFKEIAEAYDVLSDDIGGGGSGGGGSGGGGSGGGGSAAASNWKWWPGIFDGLESNWKWWPGIFD

SEQ ID NO: 101 - protein, 117 aa, Artificial
  JB1(1-67)-2XQBP1
MGKDYYQTLGLARGASDEEIKRAYRRQALRYHPDKNKEPGAEEKFKEIAEAYDVLSDPRKREIFDRYDIGGGGSGGGGSGGGGSGGGGSAAASNWKWWPGIFDGLESNWKWWPGIFD

SEQ ID NO: 102 - protein, 158 aa, Artificial
  JB1(1-108)-2XQBP1
MGKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGDIGGGGSGGGGSGGGGSGGGGSAAASNWKWWPGIFDGLESNWKWWPGIFD

SEQ ID NO: 103 - protein, 105 aa, Artificial
  JB1-2XQBP1 (no flexible linker)
MGKDYYQTLGLARGASDEEIKRAYRRQALRYHPDKNKEPGAEEKFKEIAEAYDVLSDPRKREIFDRYGEEGLKGSDIAAASNWKWWPGIFDGLESNWKWWPGIFD

SEQ ID NO: 104 - protein, 110 aa, Artificial
  JB1-2XQBP1 (rigid linker)
MGKDYYQTLGLARGASDEEIKRAYRRQALRYHPDKNKEPGAEEKFKEIAEAYDVLSDPRKREIFDRYGEEGLKGSDIEAAAKAAASNWKWWPGIFDGLESNWKWWPGIFD

SEQ ID NO: 105 - protein, 121 aa, Artificial
  JB2-2XQBP1
MASYYEILDVPRSASADDIKKAYRRKALQWHPDKNPDNKEFAEKKFKEVAEAYEVLSDKHKREIYDRYGREDIGGGGSGGGGSGGGGSGGGGSAAASNWKWWPGIFDGLESNWKWWPGIFD

SEQ ID NO: 106 - protein, 119 aa, Artificial
  JB6-2XQBP1
MVDYYEVLGVQRHASPEDIKKAYRKLALKWHPDKNPENKEEAERKFKQVAEAYEVLSDAKKRDIYDKYGDIGGGGSGGGGSGGGGSGGGGSAAASNWKWWPGIFDGLESNWKWWPGIFD

SEQ ID NO: 107 - protein, 116 aa, Artificial
  JC6-2XQBP1
MTKWKPVGMADLVTPEQVKKVYRKAVLVVHPDKATGQPYEQYAKMIFMELNDAWSEFENQGQKPLYDIGGGGSGGGGSGGGGSGGGGSAAASNWKWWPGIFDGLESNWKWWPGIFD

SEQ ID NO: 108 - protein, 125 aa, Artificial
  JB1-2XQBP2
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAHWWRSWYSDSVGLEHWWRSWYSDSV

SEQ ID NO: 109 - protein, 349 aa, Artificial
  JB1-scFv(MW1)
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAQVQLQESGGGLVQPGGSLKLSCAASGFTFRDYYMYWVRQTPEKRLEWVAFISNGGGSTYYPDTVKGRFTISRDNAKNTLYLQMSRLKSEDTAMYYCARGRGYVWFAYWGQGTTVTVSSGGGGSGGGGSGGGGSQLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 110 - protein, 125 aa, Artificial
  JB1-polyQ(25)
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAQQQQQQQQQQQQQQQQQQQQQQQQQ

SEQ ID NO: 111 - protein, 121 aa, Artificial
  JB2-2XQBP2
MASYYEILDVPRSASADDIKKAYRRKALQWHPDKNPDNKEFAEKKFKEVAEAYEVLSDKHKREIYDRYGREDIGGGGSGGGGSGGGGSGGGGSAAAHWWRSWYSDSVGLEHWWRSWYSDSV

SEQ ID NO: 112 - protein, 119 aa, Artificial
  JB6-2XQBP2
MVDYYEVLGVQRHASPEDIKKAYRKLALKWHPDKNPENKEEAERKFKQVAEAYEVLSDAKKRDIYDKYGDIGGGGSGGGGSGGGGSGGGGSAAAHWWRSWYSDSVGLEHWWRSWYSDSV

SEQ ID NO: 113 - protein, 116 aa, Artificial
  JC6-2XQBP2
MTKWKPVGMADLVTPEQVKKVYRKAVLVVHPDKATGQPYEQYAKMIFMELNDAWSEFENQGQKPLYDIGGGGSGGGGSGGGGSGGGGSAAAHWWRSWYSDSVGLEHWWRSWYSDSV

SEQ ID NO: 114 - protein, 345 aa, Artificial
  JB2-scFv(MW1)
MASYYEILDVPRSASADDIKKAYRRKALQWHPDKNPDNKEFAEKKFKEVAEAYEVLSDKHKREIYDRYGREDIGGGGSGGGGSGGGGSGGGGSAAAQVQLQESGGGLVQPGGSLKLSCAASGFTFRDYYMYWVRQTPEKRLEWVAFISNGGGSTYYPDTVKGRFTISRDNAKNTLYLQMSRLKSEDTAMYYCARGRGYVWFAYWGQGTTVTVSSGGGGSGGGGSGGGGSQLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 115 - protein, 343 aa, Artificial
  JB6-scFv(MW1)
MVDYYEVLGVQRHASPEDIKKAYRKLALKWHPDKNPENKEEAERKFKQVAEAYEVLSDAKKRDIYDKYGDIGGGGSGGGGSGGGGSGGGGSAAAQVQLQESGGGLVQPGGSLKLSCAASGFTFRDYYMYWVRQTPEKRLEWVAFISNGGGSTYYPDTVKGRFTISRDNAKNTLYLQMSRLKSEDTAMYYCARGRGYVWFAYWGQGTTVTVSSGGGGSGGGGSGGGGSQLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 116 - protein, 340 aa, Artificial
  JC6-scFv(MW1)
MTKWKPVGMADLVTPEQVKKVYRKAVLVVHPDKATGQPYEQYAKMIFMELNDAWSEFENQGQKPLYDIGGGGSGGGGSGGGGSGGGGSAAAQVQLQESGGGLVQPGGSLKLSCAASGFTFRDYYMYWVRQTPEKRLEWVAFISNGGGSTYYPDTVKGRFTISRDNAKNTLYLQMSRLKSEDTAMYYCARGRGYVWFAYWGQGTTVTVSSGGGGSGGGGSGGGGSQLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 117 - protein, 121 aa, Artificial
  JB2-polyQ(25)
MASYYEILDVPRSASADDIKKAYRRKALQWHPDKNPDNKEFAEKKFKEVAEAYEVLSDKHKREIYDRYGREDIGGGGSGGGGSGGGGSGGGGSAAAQQQQQQQQQQQQQQQQQQQQQQQQQ

SEQ ID NO: 118 - protein, 119 aa, Artificial
  JB6-polyQ(25)
MVDYYEVLGVQRHASPEDIKKAYRKLALKWHPDKNPENKEEAERKFKQVAEAYEVLSDAKKRDIYDKYGDIGGGGSGGGGSGGGGSGGGGSAAAQQQQQQQQQQQQQQQQQQQQQQQQQ

SEQ ID NO: 119 - protein, 116 aa, Artificial
  JC6-polyQ(25)
MTKWKPVGMADLVTPEQVKKVYRKAVLVVHPDKATGQPYEQYAKMIFMELNDAWSEFENQGQKPLYDIGGGGSGGGGSGGGGSGGGGSAAAQQQQQQQQQQQQQQQQQQQQQQQQQ

SEQ ID NO: 120 - protein, 339 aa, Artificial
  JB1-scFv(MW7)
MASYYEILDVPRSASADDIKKAYRRKALQWHPDKNPDNKEFAEKKFKEVAEAYEVLSDKHKREIYDRYGREDIGGGGSGGGGSGGGGSGGGGSAAAQVKLQESGGGLVQPGGSMKLSCAASGFTFSDAWMDWVRQSPEKGLSGVAEIRSKANNHATYYAESVKGRFTISRDDSKSSVYLQMNSLRAEDTGIYYCIYAGFAYWGQGTTVTVSSGGGGSGGGGSGGGGSDIELTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPWTFGGGTKLEI

SEQ ID NO: 121 - protein, 214 aa, Artificial
  JB1-Happ1
MASYYEILDVPRSASADDIKKAYRRKALQWHPDKNPDNKEFAEKKFKEVAEAYEVLSDKHKREIYDRYGREDIGGGGSGGGGSGGGGSGGGGSAAAMQSVLTQPPSASGTPGQRVTISCSGSSSNIGSNYVYWYQQLPGTAPKLLIYRNNQRPSGVPDRFSGSKSGTSASLAISGLRPEDEADYYCAAWDDSLCVALVFGGGTNGGGGVDGTAG

SEQ ID NO: 122 - protein, 139 aa, Artificial
  JB1-QBP1-QBP1 (1)
MGKDYYQTLGLARGASDEEIKRAYRRQALRYHPDKNKEPGAEEKFKEIAEAYDVLSDPRKREIFDRYGEEGLKGSGGGGSGGGGSGGGGSGGGGSAAASNWKWWPGIFDLEGDYKDDDDKGSRAAASNWKWWPGIFDLE

SEQ ID NO: 123 - protein, 104 aa, Artificial
  JB1-QBP1-QBP1 (2)
MGKDYYQTLGLARGASDEEIKRAYRRQALRYHPDKNKEPGAEEKFKEIAEAYDVLSDPRKREIFDRYGEEGLKGSSNWKWWPGIFDEAAAKSNWKWWPGIFDLE

SEQ ID NO: 124 - protein, 104 aa, Artificial
  JB1-QBP1-QBP1 (3)
MGKDYYQTLGLARGASDEEIKRAYRRQALRYHPDKNKEPGAEEKFKEIAEAYDVLSDPRKREIFDRYGEEGLKGSSNWKWWPGIFDGGGGSSNWKWWPGIFDLE

SEQ ID NO: 125 - protein, 109 aa, Artificial
  JB1-QBP1-QBP1 (4)
MGKDYYQTLGLARGASDEEIKRAYRRQALRYHPDKNKEPGAEEKFKEIAEAYDVLSDPRKREIFDRYGEEGLKGSGGGGSSNWKWWPGIFDEAAAKSNWKWWPGIFDLE

SEQ ID NO: 126 - protein, 109 aa, Artificial
  JB1-QBP1-QBP1 (5)
MGKDYYQTLGLARGASDEEIKRAYRRQALRYHPDKNKEPGAEEKFKEIAEAYDVLSDPRKREIFDRYGEEGLKGSGGGGSSNWKWWPGIFDGGGGSSNWKWWPGIFDLE

SEQ ID NO: 127 - protein, 109 aa, Artificial
  JB1-QBP1-QBP1 (6)
MGKDYYQTLGLARGASDEEIKRAYRRQALRYHPDKNKEPGAEEKFKEIAEAYDVLSDPRKREIFDRYGEEGLKGSEAAAKSNWKWWPGIFDEAAAKSNWKWWPGIFDLE

SEQ ID NO: 128 - protein, 109 aa, Artificial
  JB1-QBP1-QBP1 (7)
MGKDYYQTLGLARGASDEEIKRAYRRQALRYHPDKNKEPGAEEKFKEIAEAYDVLSDPRKREIFDRYGEEGLKGSEAAAKSNWKWWPGIFDGGGGSSNWKWWPGIFDLE

SEQ ID NO: 129 - protein, 107 aa, Artificial
  JB1-QBP1-QBP1 (8)
MGKDYYQTLGLARGASDEEIKRAYRRQALRYHPDKNKEPGAEEKFKEIAEAYDVLSDPRKREIFDRYGEEGLKGSEAAAKSNWKWWPGIFDEAAAKSNWKWWPGIFD

SEQ ID NO: 130 - protein, 107 aa, Artificial
  JB1-QBP1-QBP1 (9)
MGKDYYQTLGLARGASDEEIKRAYRRQALRYHPDKNKEPGAEEKFKEIAEAYDVLSDPRKREIFDRYGEEGLKGSEAAAKSNWKWWPGIFDGGGGSSNWKWWPGIFD

SEQ ID NO: 131 - protein, 109 aa, Artificial
  JB1-QBP1-QBP1 (10)
MGKDYYQTLGLARGASDEEIKRAYRRQALRYHPDKNKEPGAEEKFKEIAEAYDVLSDPRKREIFDRYGEEGLKGSSNWKWWPGIFDGGGGSGGGGSSNWKWWPGIFDLE

SEQ ID NO: 132 - protein, 109 aa, Artificial
  JB1-QBP1-QBP1 (11)
MGKDYYQTLGLARGASDEEIKRAYRRQALRYHPDKNKEPGAEEKFKEIAEAYDVLSDPRKREIFDRYGEEGLKGSSNWKWWPGIFDEAAAKEAAAKSNWKWWPGIFDLE

SEQ ID NO: 133 - protein, 114 aa, Artificial
  JB1-QBP1-QBP1 (12)
MGKDYYQTLGLARGASDEEIKRAYRRQALRYHPDKNKEPGAEEKFKEIAEAYDVLSDPRKREIFDRYGEEGLKGSGGGGSSNWKWWPGIFDGGGGSGGGGSSNWKWWPGIFDLE

SEQ ID NO: 134 - protein, 114 aa, Artificial
  JB1-QBP1-QBP1 (13)
MGKDYYQTLGLARGASDEEIKRAYRRQALRYHPDKNKEPGAEEKFKEIAEAYDVLSDPRKREIFDRYGEEGLKGSGGGGSSNWKWWPGIFDEAAAKEAAAKSNWKWWPGIFDLE

SEQ ID NO: 135 - protein, 114 aa, Artificial
  JB1-QBP1-QBP1 (14)
MGKDYYQTLGLARGASDEEIKRAYRRQALRYHPDKNKEPGAEEKFKEIAEAYDVLSDPRKREIFDRYGEEGLKGSEAAAKSNWKWWPGIFDGGGGSGGGGSSNWKWWPGIFDLE

SEQ ID NO: 136 - protein, 114 aa, Artificial
  JB1-QBP1-QBP1 (15)
MGKDYYQTLGLARGASDEEIKRAYRRQALRYHPDKNKEPGAEEKFKEIAEAYDVLSDPRKREIFDRYGEEGLKGSEAAAKSNWKWWPGIFDEAAAKEAAAKSNWKWWPGIFDLE

SEQ ID NO: 137 - protein, 134 aa, Artificial
  JB1-QBP1-QBP1 (16)
MGKDYYQTLGLARGASDEEIKRAYRRQALRYHPDKNKEPGAEEKFKEIAEAYDVLSDPRKREIFDRYGEEGLKGSGGGGSGGGGSGGGGSGGGGSAAASNWKWWPGIFDLEGGGGSSRAAASNWKWWPGIFDLE

SEQ ID NO: 138 - protein, 134 aa, Artificial
  JB1-QBP1-QBP1 (17)
MGKDYYQTLGLARGASDEEIKRAYRRQALRYHPDKNKEPGAEEKFKEIAEAYDVLSDPRKREIFDRYGEEGLKGSGGGGSGGGGSGGGGSGGGGSAAASNWKWWPGIFDLEEAAAKSRAAASNWKWWPGIFDLE

SEQ ID NO: 139 - protein, 111 aa, Artificial
  JB1-QBP1-QBP1 (18)
MGKDYYQTLGLARGASDEEIKRAYRRQALRYHPDKNKEPGAEEKFKEIAEAYDVLSDPRKREIFDRYGEEGLKGSEAAAKSNWKWWPGIFDLEGGGGSSNWKWWPGIFDLE

SEQ ID NO: 140 - protein, 111 aa, Artificial
  JB1-QBP1-QBP1 (19)
MGKDYYQTLGLARGASDEEIKRAYRRQALRYHPDKNKEPGAEEKFKEIAEAYDVLSDPRKREIFDRYGEEGLKGSGGGGSSNWKWWPGIFDLEGGGGSSNWKWWPGIFDLE

SEQ ID NO: 141 - protein, 149 aa, Artificial
  Flag-QBP1-JB1-QBP1-Flag
MDYKDDDDKGGTGSSNWKWWPGIFDEFMGKDYYQTLGLARGASDEEIKRAYRRQALRYHPDKNKEPGAEEKFKEIAEAYDVLSDPRKREIFDRYGEEGLKGSGGGGSGGGGSGGGGSGGGGSAAASNWKWWPGIFDLESRGDYKDDDDK

SEQ ID NO: 142 - protein, 106 aa, Artificial
  QBP1-JB1-QBP1 (1)
MSNWKWWPGIFDGKDYYQTLGLARGASDEEIKRAYRRQALRYHPDKNKEPGAEEKFKEIAEAYDVLSDPRKREIFDRYGEEGLKGSSNWKWWPGIFDGDYKDDDDK

SEQ ID NO: 143 - protein, 105 aa, Artificial
  QBP1-JB1-QBP1 (2)
MSNWKWWPGIFDLEGKDYYQTLGLARGASDEEIKRAYRRQALRYHPDKNKEPGAEEKFKEIAEAYDVLSDPRKREIFDRYGEEGLKGSGGGSSNWKWWPGIFDLE

SEQ ID NO: 144 - protein, 201 aa, Artificial
  JB1-JB1-QBP1
MGKDYYQTLGLARGASDEEIKRAYRRQALRYHPDKNKEPGAEEKFKEIAEAYDVLSDPRKREIFDRYGEEGLKGSGSEFMGKDYYQTLGLARGASDEEIKRAYRRQALRYHPDKNKEPGAEEKFKEIAEAYDVLSDPRKREIFDRYGEEGLKGSGGGGSGGGGSGGGGSGGGGSAAASNWKWWPGIFDLESRGDYKDDDDK

SEQ ID NO: 145 - protein, 212 aa, Artificial
  JB1-QBP1-JB1-QBP1
MGKDYYQTLGLARGASDEEIKRAYRRQALRYHPDKNKEPGAEEKFKEIAEAYDVLSDPRKREIFDRYGEEGLKGSGSSNWKWWPGIFDEFMGKDYYQTLGLARGASDEEIKRAYRRQALRYHPDKNKEPGAEEKFKEIAEAYDVLSDPRKREIFDRYGEEGLKGSGGGGSGGGGSGGGGSGGGGSAAASNWKWWPGIFDLESRGDYKDDDDK

SEQ ID NO: 146 - protein, 137 aa, Artificial
  JB6-QBP1-QBP1
MVDYYEVLGVQRHASPEDIKKAYRKLALKWHPDKNPENKEEAERKFKQVAEAYEVLSDAKKRDIYDKYGKEGLNGGDIGGGGSGGGGSGGGGSAAASNWKWWPGIFDLEGDYKDDDDKGSRAAASNWKWWPGIFDLE

SEQ ID NO: 147 - protein, 136 aa, Artificial
  JB13-QBP1-QBP1
MGQDYYSVLGITRNSEDAQIKQAYRRLALKHHPLKSNEPSSAEIFRQIAEAYDVLSDPMKRGIYDKFGEEGLKGGDIGGGGSGGGGSGGGGSAAASNWKWWPGIFDLEGDYKDDDDKGSRAAASNWKWWPGIFDLE

SEQ ID NO: 148 - protein, 136 aa, Artificial
  JA1-QBP1-QBP1
MVKETTYYDVLGVKPNATQEELKKAYRKLALKYHPDKNPNEGEKFKQISQAYEVLSDAKKRELYDKGGEQAIKEGDIGGGGSGGGGSGGGGSAAASNWKWWPGIFDLEGDYKDDDDKGSRAAASNWKWWPGIFDLE

SEQ ID NO: 149 - protein, 133 aa, Artificial
  JC7-QBP1-QBP1
MDYYKILGVDKNASEDEIKKAYRKRALMHHPDRHSGASAEVQKEEEKKFKEVGEAFTILSDPKKKTRYDSGQDIGGGGSGGGGSGGGGSAAASNWKWWPGIFDLEGDYKDDDDKGSRAAASNWKWWPGIFDLE

SEQ ID NO: 150 - protein, 133 aa, Artificial
  DnaJ-QBP1-QBP1
MAKQDYYEILGVSKTAEEREIRKAYKRLAMKYHPDRNQGDKEAEAKFKEIKEAYEVLTDSQKRAAYDQYGHADIGGGGSGGGGSGGGGSAAASNWKWWPGIFDLEGDYKDDDDKGSRAAASNWKWWPGIFDLE

SEQ ID NO: 151 - protein, 126 aa, Artificial
  SV40-QBP1-QBP1
MQLMDLLGLERSAWGNIPLMRKAYLKKCKEFHPDKGGDEEKMKKMNTLYKKMEDGVKYAHQPDFGDIGGGGSGGGGSGGGGSAAASNWKWWPGIFDLEGDYKDDDDKGSRAAASNWKWWPGIFDLE

SEQ ID NO: 152 - protein, 111 aa, Artificial
  JB1QBP1 (1)
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFD

SEQ ID NO: 153 - protein, 88 aa, Artificial
  JB1QBP1 (2)
MGKDYYQTLGLARGASDEEIKRAYRRQALRYHPDKNKEPGAEEKFKEIAEAYDVLSDPRKREIFDRYGEEGLKGSSNWKWWPGIFDLE

SEQ ID NO: 154 - protein, 92 aa, Artificial
  JB1QBP1 (3)
MGKDYYQTLGLARGASDEEIKRAYRRQALRYHPDKNKEPGAEEKFKEIAEAYDVLSDPRKREIFDRYGEEGLKGSGGGSSNWKWWPGIFDLE

SEQ ID NO: 155 - protein, 93 aa, Artificial
  JB1QBP1 (4)
MGKDYYQTLGLARGASDEEIKRAYRRQALRYHPDKNKEPGAEEKFKEIAEAYDVLSDPRKREIFDRYGEEGLKGSEAAAKSNWKWWPGIFDLE

SEQ ID NO: 156 - protein, 86 aa, Artificial
  JB1QBP1 (5)
MGKDYYQTLGLARGASDEEIKRAYRRQALRYHPDKNKEPGAEEKFKEIAEAYDVLSDPRKREIFDRYGEEGLKGSSNWKWWPGIFD

SEQ ID NO: 157 - protein, 90 aa, Artificial
  JB1QBP1 (5)
MGKDYYQTLGLARGASDEEIKRAYRRQALRYHPDKNKEPGAEEKFKEIAEAYDVLSDPRKREIFDRYGEEGLKGSGGGSSNWKWWPGIFD

SEQ ID NO: 158 - protein, 91 aa, Artificial
  JB1QBP1 (7)
MGKDYYQTLGLARGASDEEIKRAYRRQALRYHPDKNKEPGAEEKFKEIAEAYDVLSDPRKREIFDRYGEEGLKGSEAAAKSNWKWWPGIFD

SEQ ID NO: 159 - protein, 17 aa, Artificial
  MGVKVLFALICIAVAEA
MGVKVLFALICIAVAEA

SEQ ID NO: 160 - protein, 19 aa, Artificial
  MAPVQLLGLLVLFLPAMRC
MAPVQLLGLLVLFLPAMRC

SEQ ID NO: 161 - protein, 19 aa, Artificial
  MAVLGLLFCLVTFPSCVLS
MAVLGLLFCLVTFPSCVLS

SEQ ID NO: 162 - protein, 232 aa, Artificial
  Epitope
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 8 aa, Artificial
  DYKDDDDK
DYKDDDDK

SEQ ID NO: 164 - protein, 6 aa, Artificial
  HHHHHH
HHHHHH

SEQ ID NO: 165 - protein, 10 aa, Artificial
  EQKLISEEDL
EQKLISEEDL

SEQ ID NO: 166 - protein, 9 aa, Artificial
  YPYDVPDYA
YPYDVPDYA

SEQ ID NO: 167 - protein, 14 aa, Artificial
  GKPIPNPLLGLDST
GKPIPNPLLGLDST

SEQ ID NO: 168 - protein, 220 aa, Artificial
  Epitopes
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 169 - protein, 341 aa, Artificial
  GFP-HTTQ23
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGRTQISSSSFEFATLEKLMKAFESLKSFQQQQQQQQQQQQQQQQQQQQQQQPPPPPPPPPPPQLPQPPPQAQPLLPQPQPPPPPPPPPPGPAVAEEPLHRP

SEQ ID NO: 170 - protein, 392 aa, Artificial
  GFP-HTTQ74
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGRTQISSSSFEFATLEKLMKAFESLKSFQQQQQQQQQQQQQQQQQQQQQQQQQQQQQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRP